(19) United States
(12) Patent Application Publication
(10) Pub. No.: US 2024/0272156 A1
(43) Pub. Date: Aug. 15, 2024

(54) COMPOSITIONS AND METHODS FOR IDENTIFYING AND ISOLATING HUMAN HEMATOPOIETIC STEM AND PROGENITOR CELLS

(71) Applicant: THE BRIGHAM AND WOMEN'S HOSPITAL, Boston, MA (US)

(72) Inventor: Robert Sackstein, Pinecrest, FL (US)

(21) Appl. No.: 18/591,921

(22) Filed: Feb. 29, 2024

Related U.S. Application Data
(63) Continuation of application No. PCT/US2024/011599, filed on Jan. 16, 2024.
(60) Provisional application No. 63/479,853, filed on Jan. 13, 2023.

(51) Int. Cl. G01N 33/569; C12N 5/0775; C12Q 1/6851; C12Q 1/6881

(52) U.S. Cl. CPC G01N 33/56966; C12N 5/0662; C12Q 1/6851; C12Q 1/6881; C12Q 2600/158

(57) ABSTRACT

The present disclosure provides, inter alia, methods for selecting hematopoietic stem/progenitor cells (HSPCs) from a heterogenous population of nucleated cells, and compositions comprising selected cells useful for treatment of a medical condition. In some embodiments, the cells are selected for the characteristic markers sialylated Lewis X (sLeX), CD34+, and CD38−. In some embodiments, the selected cells comprise hematopoietic stem cells (HSCs).

### STATEMENT OF GOVERNMENT FUNDING

This invention was made with government support under grant PO1 HL107146 awarded by the National Institutes of Health. The government has certain rights in this invention.

### FIELD OF THE INVENTION

This disclosure relates to improved compositions and methods for characterizing, identifying, and isolating operationally distinct subsets of cells by identifying a distinct pattern of expression of glycans on the cell surface.

### BACKGROUND OF THE INVENTION

In all mammalian cells, the cell surface is comprised of a complex tapestry of glycans, presented in the form of glycoproteins and glycolipids. This florid weave of carbohydrate structures is known as the “glycocalyx”, and the composition of this sugar coat is unique to any given cell type. However, due to significant technical challenges in characterizing these structures, compounded by difficulties in obtaining adequate amounts of glycans for analysis (especially from limited quantities of biologic specimens), little is known about how distinct glycan determinants that comprise the glycocalyx vary as to diverse cell types or as to developmental differentiation of cells in a stage- and lineage-specific fashion.

In many cases, the discrete compositional combination and relevant linkages (i.e., stereospecific localization) of certain monosaccharides (i.e., core sugar units) covalently clustered into oligosaccharides or polysaccharides comprising the glycocalyx imparts a critical biologic property. Indeed, several key biologic effects are exclusively mediated by glycan determinants/composition. For example, the amount of sialic acid (also known as “N-acetyl-neuraminic acid”, or, more simply, “neuraminic acid”) found on the surface of a blood leukocyte or a platelet dictates whether that cell will be destroyed (cleared) by the reticulo-endothelial system. Besides sialylation, the content and location of fucoses on the cell surface also imparts distinguishing biology. Importantly, a sialylated and fucosylated (i.e., “sialofucosylated”) tetrasaccharide displayed on cell surfaces called “sialylated Lewis X” or “sialyl-Lewis X” (abbreviated as “sLeX”; also known as “CD15s”: NeuAc-α(2,3)-Gal-β(1,4)-[Fuc-α(1,3)]-GlcNAc-R, where “NeuAc” is sialic acid, “Gal” is galactose, “Fuc” is fucose, “GlcNAc” is N-acetylglucosamine, and “R” is carbohydrate chain covalently attached to a protein or lipid scaffold) is an operationally critical cell surface glycan motif because it serves as the canonical binding determinant for E-selectin (CD62E), a C-type lectin expressed on endothelial cells. Interactions between vascular E-selectin and sLeX displayed on the surface of circulating cells mediates hemodynamic shear-resistant adhesion onto the endothelial surface, the initial step in extravasation of blood-borne cells (Butcher, 1991; Sackstein, 2004; Springer, 1994).

The sLeX tetrasaccharide motif is localized at the termini of glycan chains displayed on membrane glycoproteins and glycolipids, and consists of a backbone “Type 2-lactosamine unit” (Type 2-LacNAc: Gal-β(1,4)-GlcNAc-R; this core disaccharide unit is also called a “neutral” Type 2-LacNAc), whereupon sialic acid is α(2,3)-linked to Gal (this core trisaccharide is known as a “Type 2-α(2,3)-sialylated lactosamine” (Type 2-α(2,3)-sialylLacNAc: NeuAc-α(2,3)-Gal-β(1,4)-GlcNAc-R) and fucose is α(1,3)-linked to GlcNAc (again, sLeX: NeuAc-α(2,3)-Gal-β(1,4)-[Fuc-α(1,3)]-GlcNAc-R). Biosynthesis of sLeX proceeds via the action of pertinent glycosyltransferases within the Golgi, and occurs in a defined step-wise fashion whereby a Type 2-lactosamine unit must first undergo placement of sialic acid in α(2,3)-linkage to the terminal Gal (a reaction catalyzed by α(2,3)-sialyltransferases), then followed by placement of fucose in α(1,3)-linkage to the GlcNAc within the α(2,3)-sialylated Type 2-lactosamine unit (a reaction catalyzed by α(1,3)-fucosyltransferases). If α(1,3)-fucosylation precedes the α(2,3)-sialylation, the trisaccharide structure known as “Lewis X” (LeX; also known as “CD15”) is created: Gal-β(1,4)-[Fuc-α(1,3)]-GlcNAc-R. This is an important caveat, as there is no sialyltransferase that can sialylate a Lewis X structure to create sLeX (i.e., biosynthetically, creation of LeX blocks construction of sLeX) (Sackstein, 2009).

E-selectin is characteristically an inducible endothelial molecule (expression is not constitutive, and is induced by trauma, ischemia, bacterial infections (e.g., LPS), and by inflammatory cytokines such as interleukin-1 (IL-1) and tumor necrosis factor (TNF)), and it is a principal mediator of leukocyte recruitment at inflammatory sites. However, notably, E-selectin is constitutively expressed by specialized marrow sinusoidal endothelial cells (Schweitzer et al., 1996; Sipkins et al., 2005). Intravital microscopy studies in mice of intravascularly administered human and mouse hematopoietic stem/progenitor cells (HSPCs) have provided direct evidence that E-selectin receptor/ligand interactions within marrow sinusoidal beds promote recruitment of circulating HSPCs into bone marrow (Katayama et al., 2003; Schweitzer et al., 1996; Sipkins et al., 2005). Thus, sLeX has garnered interest for its role in mediating the migration of HSPCs to bone marrow, (“osteotropism”), a process fundamental to both embryonic hematopoietic development and the success of clinical hematopoietic stem cell transplantation (HSCT; historically called “bone marrow transplant”) (Sackstein, 2016). Surprisingly, however, no studies to date have undertaken a systematic assessment of sLeX expression on human HSPCs, and, most importantly, knowledge of the level of sLeX expression on human hematopoietic stem cells (HSCs) is grossly incomplete; indeed, a recent review summarizing all current knowledge regarding cell surface markers that define human HSCs contains no mention of sLeX, nor, for that matter, of any gly can structure (Rix et al, 2022). Importantly, recent studies in mice indicate that sLeX expression levels— and, more critically, the level of E-selectin binding—is conspicuously lower on HSCs than on more mature hematopoietic progenitors; indeed, a comprehensive study of sLeX expression in murine HSPCs has shown that the most primitive mouse HSPCs, the “long-term HSCs” (LT-HSCs), are relatively deficient in sLeX expression (Al-Amoodi et al., 2022). Given that cell surface sLeX display is a mediator of HSPC homing to marrow, the conspicuously low sLeX expression observed on murine HSCs could suggest that these cells innately lack a high capacity for osteotropism. However, it is important to note that a variety of HSPC surface proteins including CXCR4, β1 integrins VLA-4 and VLA-5, and β2 integrin LFA-1 (Frenette et al., 1998; Hidalgo and Frenette, 2005; Katayama et al., 2003; Peled et al., 1999; Peled et al., 2000) have each been found to cooperatively orchestrate marrow homing (reviewed in (Lapidot et al., 2005)), raising the notion that a disproportionate abundance of one (or combination) of these cooperating proteins on mouse HSCs and on human HSCs could readily obviate the contribution(s) of sLeX/E-selectin adhesive interactions in promoting HSC osteotropism (Hidalgo and Frenette, 2005).

The process of hematopoiesis is a highly ordered hierarchical system of cell proliferation and differentiation events. At the apex of this hierarchy exists the authentic hematopoietic stem cell (HSC), defined by its operational properties of self-renewal and multipotency, i.e., its capacity of differentiating into a panoply of “progeny subsets” comprising all hematopoietic progenitors capable of making all types of blood cells (including platelets). The (progressively maturing) progenitors arising from the HSC are comprised of various functional subsets that have increasingly more limited differentiation potential, and they are each characterized by the types of cells they can generate. In the mouse, studies to define the hematopoietic hierarchical roadmap have been comprehensive, and the various markers to define the murine HSC and murine hematopoietic progenitor subsets are highly validated and well-established. In stark contrast, the characterization of cell surface markers that can be used to identify the various components of the human hematopoietic hierarchy are still evolving, and there still remains a pressing need to develop novel approaches to more efficiently and specifically define the human HSC as well as stage- and lineage-specific, oligopotent human hematopoietic progenitors.

The glycoprotein known as “CD34” is a principal marker of human HSPCs and its expression encompasses an assorted population of hematopoietic progenitors ranging from the HSC to a series of more differentiated oligopotent cells. The glycoprotein known as “CD38” also has utility in defining human HSPCs, and, importantly, essentially all primitive hematopoietic progenitors (including the human HSC) reside within the CD34+/CD38− fraction of hematopoietic cells derived from human marrow and human cord blood. Throughout the 1990s and early 2000s, the hematopoietic cell population that lacked expression of all markers associated with lineage-specific differentiation/commitment (i.e., the Lineage−subset (“Lin−cells”)) was used in combination with the CD34+/CD38− phenotype (thus, CD34+/CD38−/Lin−cells) to obtain a human hematopoietic population grossly enriched for HSCs. Then, in 2011, it was reported that a pentad-combination of membrane markers consisting of the following pattern—CD34⁺CD38⁻CD90⁺CD45RA⁻CD49f⁺—can serve to identify and isolate the authentic human HSC (Notta et al., 2011), and, subsequently, this 5-marker phenotype of the human HSC has been well-validated in numerous studies and currently serves as the reference standard (the “conventional” phenotypic description of the human HSC (“HSC_{conv}”)). Against this backdrop, multiple studies have indicated that sLeX expression on native human CD34+ HSPCs is widely heterogeneous (with ˜30% of cells lacking sLeX expression altogether) (Olweus et al., 1994), and that primitive HSPCs (CD34⁺CD38⁻ cells) within human umbilical cord blood (UCB) generally lack sLeX expression (Hidalgo and Frenette, 2005; Xia et al., 2004). A greater understanding of the subsets of human HSPCs that either maintain/preserve sLeX expression or choose not to express this determinant, the relative level(s) of expression of sLeX among those subsets of HSPCs that express this motif, altogether with information on the levels of expression of its precursor Type 2-lactosaminyl glycans, could further inform on operationally distinct hematopoietic progenitor subpopulations. However, the inherent limitations in acquiring sufficient numbers of human cells comprising discrete hierarchical hematopoietic networks, compounded by inherent limitations of techniques to assess the expression levels of relevant cell surface glycans, pose significant challenges to achieving a comprehensive understanding of how stage- and lineage-specific variations in expression of lactosaminyl glycans could serve as phenotypic markers of pertinent human hematopoietic progenitor subsets.

The need to continue to define new phenotypic markers of subsets of cells and, thereby, continue to improve methods to collect the target cell subset(s) is becoming increasingly more imperative in light of advances in the field of cell therapy. This necessity is being additionally fueled by advances in genetic manipulation that offer cure of various genetic diseases, yet it is clear that this promise can only be realized by improvements in methods to effectively and efficiently enrich or isolate the cell type(s) necessitating such genetic correction. As one pertinent example, the HSC is the optimal target cell type for curative-intent genetic therapy for patients suffering from hemoglobinopathies or immune deficiencies: the in vitro correction of the genetic defect within the patient's own HSC population, then followed by an autologous HSCT, would ensure that all blood cell types derived from the genetically-corrected HSC would harbor the pertinent genetic correction. But, clearly, it is first necessary to harness sufficient numbers of HSCs from the affected patient for subsequent in vitro genetic manipulation, a process critically dependent on the ability to accurately and readily detect and collect the requisite quantities of HSCs in hematopoietic tissue (e.g., marrow, spleen, or fetal liver) and/or in blood. Beyond utility in assessing the numerical content of HSCs from primary human tissue sources, this information would be valuable in both quantifying and operationally assessing HSCs within HSPC populations that have been manipulated and/or expanded in vitro (i.e., as cultured human HSPCs), or in cases where HSPCs are derived from genetically-modified and/or epigenetically-modified precursor cells or from pluripotent stem cell sources (e.g., HSPCs derived from human induced-pluripotent stem cells or from human embryonic stem cells).

At present, there are two principal technologies for characterization of glycan structures: mass spectrometry (MS) and nuclear magnetic resonance (NMR). Though powerful tools, these methods entail very expensive, specialized equipment necessitating considerable operational expertise, they involve tedious sample preparation, and they necessitate substantial amounts of both the starting material and the derivative analyte. Moreover, these methods cannot quantify expression levels of a given glycan, especially not on cell surfaces, and the sample quantities required for MS- or NMR-based glycan analysis precludes their applicability for characterization of glycans expressed among rare cell populations. Though flow cytometry using anti-glycan mAbs and/or lectins could provide both qualitative and quantitative information regarding the display of discrete glycan motifs on scarce cells, the current inventory of glycan-specific mAb reagents is quite limited and lectins are hindered by a general lack of both specificity and sensitivity. Importantly, at present, there are no mAbs or lectins that can specifically identify either terminal Type 2-LacNAc or terminal Type 2-α(2,3)-sialylLacNAc. To overcome this bottleneck, it is necessary to develop strategies to precisely and quantitatively identify pertinent glycan structures expressed on rare cell populations, ideally without the need for highly specialized instrumentation and expertise.

The present disclosure addresses these and other needs.

### SUMMARY OF THE INVENTION

According to some aspects, the present disclosure provides a method for characterizing, identifying, and isolating a subset of cells from a heterogenous mixture, as well as kits therefor, on the basis of a distinguishing display of a given glycan motif or a pattern of certain glycan motifs on the cell surface such as to define pertinent subpopulations of cells (a “glycosignature” or “glyco-epitope” or “glycotype” or “glycotope” of the pertinent cells). In some embodiments, a new glycosyltransferase-based glycoanalytic method (called “Glycosyltransferase Acceptor-Product Analysis” (GAP analysis)) is provided that enables precise identification on any cell population of the levels of the biosynthetic glycan precursors (i.e., the (acceptor) terminal lactosaminyl glycans) known as a “Type 2 lactosamine” (“Type 2-LacNAc”: Gal-β(1,4)-GlcNAc-R) and a “Type 2 α(2,3)-sialylated lactosamine” (“Type 2-α(2,3)-sialylLacNAc”: NeuAc-α(2,3)-Gal-β(1,4)-GlcNAc-R) that, respectively, undergo α(1,3)-fucosylation of the GlcNAc to yield the trisaccharide Type 2 lactosaminyl glycan motif known as “Lewis X” (LeX; also known as “CD15”: Gal-β(1,4)-[Fuc-α(1,3)]-GlcNAc-R)) or the tetrasaccharide Type 2 lactosaminyl glycan motif known as “sialylated Lewis X” (sLeX; also known as “CD15s”: NeuAc-α(2,3)-Gal-β(1,4)-[Fuc-α(1,3)]-GlcNAc-R)). The GAP analysis method quantifies the expression level of these acceptors via measurement of the level of increased expression of the LeX or sLeX glycan determinants following treatment of the surface of cells with an α(1,3)-fucosyltransferase together with a donor nucleotide fucose such as to stereospecifically install fucose in α(1,3)-linkage to GlcNAc within the target Type 2-lactosamine or Type 2 α(2,3)-sialylated lactosamine acceptors, respectively. In some embodiments, a method is provided for defining the distinct level of expression of various types of terminal lactosaminyl glycans on the surface of operationally distinct subsets of human hematopoietic stem and progenitor cells (HSPCs), such as to provide a “glycosignature” capable of characterizing, identifying, and isolating pertinent subpopulations of such cells. In some embodiments, a method is provided to enrich human hematopoietic stem cells (HSCs) on the basis of expression of sLeX. In other embodiments, a method is provided to identify and enrich operationally distinct subsets of human HSPCs based on low levels of expression of sLeX among such subpopulations. In other embodiments, the distinct patterns of expression of fucosylated lactosamines installed on the cell surface following the contacting of cells with one or more α(1,3)-fucosyltransferases together with donor nucleotide-fucose (GDP-fucose) can be used to characterize and isolate defined cell subsets within a heterogenous mixture of cells. In other embodiments, cells containing distinct biologic properties can be isolated following the fucosyltransferase-mediated enforced α(1,3)-fucosylation by enriching those target cells that have the relevantly engendered higher levels of LeX or sLeX expression. In other embodiments, stereospecific addition of a molecular tag-modified donor nucleotide-fucose (e.g., azido-modified or biotin-modified fucose-GDP) allows for subsequent identification of the installed fucose onto cell surface lactosaminyl glycans, and thus, by separating cells on the basis of the relative level(s) of the installed tagged-fucose, can be used to identify and isolate cells on the basis of the level of expression of the pertinent (underlying target) lactosaminyl glycan acceptor.

The discovery that distinct human HSPC subsets can be defined on the basis of sLeX expression streamlines the number of markers currently used to identify subsets of HPSCs, and thus would significantly increase both the efficiency and the quantitative yield of the intended subset that is sought from within a heterogenous composition of hematopoietic cells: for example, high sLeX expression on CD34+/CD38− HSPCs supplants three markers (CD90+, CD45RA−, and CD49f⁺) that are conventionally deemed necessary to isolate authentic human HSCs (i.e., HSC_{conv}). Methods employing the quantification of cell surface sLeX expression thereby allows for more robust isolation of substantially homogenous compositions of subsets of human HSPCs, including cells comprising human HSCs, GMPs, and MEPs. The human HSCs and/or other subsets of HSPCs have clinical applicability for therapy of a variety of diseases/conditions, including, but not limited to, conditions requiring the replacement of certain cell types, the regeneration of hematopoietic elements, or the engraftment of genetically-modified cells. As a set of non-limiting examples, improved isolation/collection of HSCs would be critical for success of HSCT, for treatment of aplastic anemia, for HSC-based genetic correction/gene editing of hemoglobinopathies followed by HSCT, for HSC-based genetic correction/gene editing of immune deficiency conditions followed by HSCT, or for HSC-based genetic manipulation/gene editing of cell surface molecules that allow entry and infection of pathogens into hematopoietic cells followed by HSCT (e.g., eliminating expression of CCR5 on HSCs to prevent HIV entry/infection, followed by HSCT of CCR5-deficient HSCs that would thus generate CCR5-deficient leukocytes). In addition, for example, improved methods to isolate GMPs and MEPs on basis of sLeX expression levels would be useful for treatment of delayed engraftment following HSCT and/or for treatment of marrow failure states wherein production of platelets or red cells (erythrocytes) or myeloid cells (neutrophils or monocytes) is deficient.

According to some aspects, the present disclosure provides a method for selecting one or more human hematopoietic stem/progenitor cells (HSPCs) from within a heterogenous population of lin⁻ HSPCs comprising: contacting the heterogenous population of lin⁻ HSPCs with a binding molecule for sialylated Lewis X (sLeX); measuring the amount of sLeX present on individual cells in the heterogenous population of lin⁻ HSPCs; and selecting for one or more of sLeX^{high} cells (most conveniently achieved via FACS of sLeX-stained cells) based on the level of sLeX expression within lin⁻ HSPCs, wherein the sLeX^{high} cells are the cells having the highest 15% sLeX expression level within the heterogenous population of sLeX+ lin⁻ HSPCs (i.e., the sLeX^{high} subset comprises the (top) 15% fraction of sLeX-bearing lin⁻ HSPCs that have the highest abundance/density of cell surface sLeX). In some embodiments, the method further comprises the step of selecting for CD38− cells. In some embodiments, the method further comprises the step of selecting for CD34+ cells. In some embodiments, the method further comprises the step of selecting for CD38+ cells.

In some embodiments, the heterogenous population of lin⁻ HSPCs is from bone marrow, umbilical cord blood, adult (post-natal) blood, fetal blood, fetal liver, fetal spleen, embryonic yolk sac, embryonic ventral endothelium of dorsal aorta, adult (post-natal) liver, or adult (post-natal) spleen. In some embodiments, the heterogenous population of lin⁻ HSPCs are obtained by one or more steps of depleting differentiated HSPCs expressing lineage markers (i.e., depletion of lin⁺ nucleated cells). However, notably, positive selection for CD34 expression can be used in the first stage of human HSPC enrichment from the heterogenous population (i.e., the selection of the CD34+ fraction of HSPCs without prior depletion of lin+ cells), as a CD34+ cell-selection step would inherently enrich for lin⁻ HSPCs.

In some embodiments, the selection for one or more of sLeX^{high} and CD38− cells within a population of lin⁻ HSPCs comprises one or more steps of positive selection or negative selection. In some embodiments, the selection of sLeX^{high} cells comprises one or more negative selection steps of depleting (e.g., via fluorescence-activated cell sorting (FACS) and staining of cells with fluorochrome-conjugated anti-sLeX mAb) of the heterogenous population of lin⁻ human HSPCs expressing sLeX at density levels within the lower 85% of the range of sLeX expression within the heterogenous cell population. In some embodiments, the selecting for one or more of sLeX^{high} cells comprises selecting for cells having the highest 10% of sLeX expression within the heterogenous population of cells. In some embodiments, the selecting step comprises use of a molecule that binds the glycan determinant sLeX and the anti-determinant molecule contains a selection tag whereby cells bearing the anti-determinant molecule (e.g., anti-sLeX antibody, E-selectin-Ig chimera, etc.) can then be separated. In some embodiments, the selecting step comprises use of a molecule that binds the glycan determinant sLeX that carries a functional group to allow detection and separation of cells bearing the molecule such as the use of magnetic bead-tagged anti-determinant molecules (e.g., magnetic bead-conjugated anti-sLeX antibody, magnetic bead-conjugated E-selectin-Ig chimera), biotin-tagged anti-determinant molecules (e.g., biotin-tagged anti-sLeX antibody, biotin-tagged E-selectin-Ig chimera), FACS utilizing fluorochrome-tagged anti-determinant molecules (e.g., direct (one-step) using a fluorochrome-tagged anti-determinant molecules, or indirect (two-step) using a fluorochome-tagged secondary reagent that recognizes the (primary) anti-determinant molecule (indirect (two-step) fluorochrome labelling of the cell)), chemically-modified anti-determinant molecules (e.g., anti-determinant molecule modified to contain a “clickable” chemical reagent such as an alkyne or azide modification), “panning” by affixing the anti-determinant molecules to a solid support matrix, passage of cells over affinity columns containing the anti-determinant molecules attached to beads or other matrices, or other techniques providing accurate cell separation.

According to some aspects, the present disclosure provides a method for selecting one or more human hematopoietic stem/progenitor cells (HSPCs) from within a heterogenous population of lin⁻ HSPCs comprising: contacting the heterogenous population of lin⁻ HSPCs with a binding molecule for sialylated Lewis X (sLeX); measuring the amount of sLeX present on individual cells in the heterogenous population of lin⁻ HSPCs; and selecting (most conveniently via FACS) for one or more of sLeX^{low/-} cells based on absence-to-very lowest levels of sLeX expression of the cells within the pertinent population, wherein the sLeX^{low/-} cells comprise the (bottom) 15% fraction of the total heterogenous population of sLeX-stained lin⁻ HSPCs (that may variably comprise cells that express no sLeX and/or cells expressing very low levels of sLeX: those cells that by FACS comprise the 15% fraction of the entire population that have the lowest fluorescence staining level for sLeX).

In some embodiments, the method further comprises the step of selecting for CD38− cells. In some embodiments, the method further comprises the step of selecting for CD34+ cells. In some embodiments, the method further comprises the step of selecting for CD38+ cells.

In some embodiments, the heterogenous population of lin⁻ HSPCs is from bone marrow, umbilical cord blood, adult (post-natal) blood, fetal blood, fetal liver, fetal spleen, embryonic yolk sac, embryonic ventral endothelium of dorsal aorta, adult (post-natal) liver, or adult (post-natal) spleen.

In some embodiments, the selection for one or more of sLeX^{low/-}, CD34+, and CD38− cells comprises one or more steps of positive selection or negative selection to select for cells expressing the markers sLeX^{low/-}, CD34 and CD38. In some embodiments, the heterogenous population of lin⁻ HSPCs are obtained by one or more steps of depleting differentiated HSPCs expressing lineage markers (i.e., depletion of lin+ cells). In some embodiments, the selection comprises one or more negative selection steps to enrich a population of sLeX^{low/-} cells by depletion of cells expressing sLeX at cell density levels >85% of the level within the heterogenous cell population. In some embodiments, the sLeX^{low/-} cells are the cells having the lowest 10% of sLeX expression level in the heterogenous population of nucleated cells. In some embodiments, the selecting step comprises use of a molecule that binds the determinant (e.g., sLeX) and the anti-determinant molecule contains a selection tag whereby cells bearing the anti-determinant molecule (e.g., anti-sLeX antibody, E-selectin-Ig chimera, etc.) can then be separated. In some embodiments, the selecting step comprises magnetic bead-tagged anti-determinant molecules (e.g., magnetic bead-conjugated anti-sLeX antibody, magnetic bead-conjugated E-selectin-Ig chimera), biotin-tagged anti-determinant molecules (e.g., biotin-tagged anti-sLeX antibody, biotin-tagged E-selectin-Ig chimera), fluorescence-activated cell sorting (FACS) utilizing fluorochrome-tagged anti-determinant molecules (e.g., direct (one-step) using a fluorochrome-tagged anti-determinant molecule, or indirect (two-step) using a fluorochome-tagged secondary reagent that recognizes the (primary) anti-determinant molecules (indirect (two-step) fluorochrome labelling of the cell)), chemically-modified anti-determinant molecules (e.g., anti-determinant molecule modified to contain a “clickable” chemical reagent such as an alkyne or azide modification), “panning” by affixing the anti-determinant molecules to a solid support matrix, passage of cells over affinity columns containing the anti-determinant molecules attached to beads or other matrices, or other techniques providing accurate cell separation.

According to some aspects, the present disclosure provides a method for grading the level of expression of terminal α(2,3)-sialylated Type-2 lactosamine units on cells comprising the steps of: measuring the level of expression of sialylated Lewis X (sLeX) on the surface of one or more cells; contacting the one or more cells with an α(1,3)-fucosyltransferase capable of creation of sLeX from an acceptor terminal α(2,3)-sialylated Type-2 lactosamine together with a nucleotide donor sugar (GDP-fucose); measuring the level of sLeX expression on the fucosyltransferase-treated cells, wherein the increase in sLeX expression from Step (a) compared to that following Step (b) indicates the level of terminal α(2,3)-sialylated Type-2 lactosamine units on the one or more cells.

According to some embodiments, the α(1,3)-fucosyltransferase is selected from the group consisting of FTVI, FTVII, FTIII, FTV, and FTIV. In some embodiments, the GDP-fucose is modified with a selection tag that allows for separation of those cells containing the installed fucose. In some embodiments, the selection tag consists of a chemically “tagged” GDP-fucose covalently modified with a fluorochrome, a “clickable” chemical group, biotin, a radiolabel, or any other molecule covalently linked to the fucose moiety within GDP-fucose that can be used to identify the installed fucose. In some embodiments, the measuring of step (a) and step (b) comprises contacting the sLeX with a fluorescent binder and measuring mean fluorescence intensity (MFI) by flow cytometry.

According to some aspects, the present disclosure provides a method for grading the level of expression of terminal “neutral” Type-2 lactosamine units on cells comprising the steps of: measuring the level of expression of Lewis X (LeX) on the surface of one or more cells; contacting the one or more cells with an α(1,3)-fucosyltransferase capable of creating LeX from an acceptor terminal “neutral” Type-2 lactosamine together with a nucleotide donor sugar (GDP-fucose); measuring the level of LeX expression on the fucosyltransferase-treated cells, wherein the increase in LeX level expression from Step (a) compared to that following Step (b) indicates the level of terminal “neutral” Type-2 lactosamine units on the one or more cells.

In some embodiments, the α(1,3)-fucosyltransferase is selected from the group consisting of FTIX, FTVI, FTIV, FTIII, and FTV. In some embodiments, the GDP-fucose is modified with a chemical tag that allows for separation of those cells containing the installed fucose. In some embodiments, the selection tag consists of a chemically “tagged” GDP-fucose covalently modified with a fluorochrome, a “clickable” chemical group, biotin, a radiolabel, or any other molecule covalently linked to the fucose moiety within GDP-fucose that can be used to identify the installed fucose. In some embodiments, the measuring of step (a) and step (b) comprises contacting the LeX with a fluorescent binder and measuring mean fluorescence intensity (MFI) by flow cytometry.

According to some aspects, the present disclosure provides a method of selecting cells having free terminal α(2,3)-sialylated Type-2 lactosamine units comprising the steps of: contacting a population of cells with an α(1,3)-fucosyltransferase capable of creation of sLeX from an acceptor terminal α(2,3)-sialylated Type-2 lactosamine together with a chemically-tagged nucleotide donor sugar (tagged GDP-fucose); and selecting for cells having sLeX comprising the tagged-fucose within the population of cells; wherein the selected cells from step (b) comprise cells originally having free terminal α(2,3)-sialylated Type-2 lactosamine units.

In some embodiments, the α(1,3)-fucosyltransferase is selected from the group consisting of FTVI, FTVII, FTIII, FTV, and FTIV. In some embodiments, the chemical tag is selected from the group consisting of a fluorochrome, a clickable chemical group, biotin, a radiolabel, or any other molecule covalently linked to the fucose moiety within GDP-fucose that can be used to identify the installed fucose.

According to some aspects, the present disclosure provides a method of selecting cells having terminal free “neutral” Type-2 lactosamine units comprising the steps of: contacting a population of cells with an α(1,3)-fucosyltransferase capable of creation of LeX from an acceptor terminal “neutral” Type-2 lactosamine together with a tagged nucleotide donor sugar (tagged GDP-fucose); selecting for cells having LeX comprising the installed tagged fucose within the population of cells; wherein the selected cells from step (b) comprise cells originally having terminal free Type-2 lactosamine units.

In some embodiments, the α(1,3)-fucosyltransferase is selected from the group consisting of FTIX, FTVI, FTIV, FTIII, and FTV. In some embodiments, the chemical tag is selected from the group consisting of a “tagged” GDP-fucose covalently modified with a fluorochrome, a clickable group, biotin, a radiolabel, or any other molecule covalently linked to the fucose moiety within GDP-fucose that can be used to identify the installed fucose.

According to some aspects, the present disclosure provides a composition comprising the cells selected according to the methods disclosed herein. According to some embodiments, the present disclosure provides a method of treating a human subject in need thereof comprising the step of administering to the human subject a therapeutically effective amount of the cells selected according to the methods disclosed herein. In some embodiments, the cells are effective to treat one or more of hematologic diseases/disorders/conditions, genetic diseases/disorders/conditions, congenital diseases/disorders/conditions, degenerative diseases/disorders/conditions, cancerous diseases/disorders/conditions, immune diseases/disorders/conditions, drug reactions, toxin-induced injury, psychiatric diseases/disorders/conditions, vascular diseases/disorders/conditions, inflammatory diseases/disorders/conditions, iatrogenic conditions, infectious diseases/disorders/conditions, trauma, burns, ischemia/reperfusion injury, nervous system diseases/disorders/conditions, sepsis, cytokine-induced diseases/conditions/disorders, and tissue/organ failure.

According to some aspects, the present disclosure provides a kit for enriching or isolating one or more human hematopoietic stem/progenitor cells (HSPCs) from within a heterogenous population of HSPCs comprising reagents for selecting one or more of the markers sLeX, CD38, and CD34, and instructions for use thereof. In some embodiments, the kit comprises the reagents for using sLeX to detect one or more of subsets of HSPCs, such as HSCs, MEPs, GMPs, and endothelial progenitor cells (EPCs), within a population of HSPCs. In some embodiments, the kit comprises the reagents for stratifying and/or isolating subsets of HSPCs (such as HSCs, MEPs, and GMPs), as well as EPCs.

### BRIEF DESCRIPTION OF THE DRAWINGS

The patent or application file contains at least one drawing executed in color. Copies of this patent or patent application publication with color drawing(s) will be provided by the Office upon request and payment of the necessary fee.

The following drawings form part of the present specification and are included to further demonstrate certain aspects of the present invention. The invention may be better understood by reference to one or more of these drawings in combination with the detailed description of specific embodiments presented herein.

FIG. 1 shows (A) Schematic depicting the differentiation hierarchy of human HSPCs. Each node within the hierarchy represents cells at a specific developmental stage (see also Supplemental Table 1). (B-E) Phenotyping of human HSPCs. Human umbilical cord blood (UCB) and marrow-derived CD34⁺ cells were co-stained with monoclonal antibodies (mAbs) against CD34, CD38, CD90 (Thy-1), CD135, CD45RA, CD49f, sLeX ((clone HECA452), staining pattern not shown here). (B) Gates P0 and P1 represent CD34⁺CD38− and CD34⁺CD38⁺ populations respectively. (C) P0 is further partitioned into HSC_{conv}+ MPP1 (CD34⁺CD38⁻CD90⁺CD45RA⁻), MPP2 (CD34⁺CD38⁻CD90⁻CD45RA⁻), and MLP (CD34⁺CD38⁻CD90⁻CD45RA⁺). (D) HSC_{conv}+ MPP1 is further subdivided into HSC_{conv} (CD34⁺CD38⁻CD90⁺CD45RA⁻CD49f⁺), and MPP1 (CD34⁺CD38⁻CD90⁺CD45RA⁻ CD49f⁻). (E) P1 is fractionated into CMP (CD34⁺CD38⁺CD135⁺CD45RA⁻), GMP (CD34⁺CD38⁺CD135⁺CD445RA⁺), and MEP (CD34⁺CD38⁺CD135⁻CD45RA⁻) subsets. sLeX staining level was analyzed for each of these subsets.

FIG. 2 shows expression levels of integrins and CXCR4 on human HSPC subsets. (A-F) Expression of the integrin subunits (A) β1 (CD29, N=10), (B) α4 (CD49d, n=5), (C) α5 (CD49e, N=5), (D) α6 (CD49f, n=5), (E) αL (CD11a, N=5), and (F) β2 (CD18, n=4), and (G) Chemokine receptor CXCR4 (n=3) on HSPC subsets. Data are presented as mean±SD. Statistics: Repeated measures ANOVA (A, P=0.0011, B, P<0.0001, C, P=0.3327, D, C=0.0246, E, P=0.0014, F, P<0.0001, G, P=0.0007) with Tukey's multiple comparison test. (A) *P<0.05, **P<0.01, ***P<0.001, ****P<0.0001. Square brackets indicate data points compared. (D) *P<0.05, **P<0.01. Square brackets indicate data points compared. #P<0.05 indicates that these data points are different from all other points, but these values are not statistically different from each other. (E) **P<0.01. Square brackets indicate data points compared. †, #P<0.0001, indicate that the designated data points are different from all other data points. † and # are significantly different from each other (P<0.05). (F) †, #P<0.0001, indicate that the designated data points are different from all other data points. † and # are significantly different from each other (P<0.05). (G) #P<0.0001 compared all other data points. (H) Normalized transmigration activity of HSPCs across CXCL12 (stromal derived factor-1 (SDF-1)) gradient. CXCR4 antagonist AMD3100 was used to confirm that the migration was CXCR4-mediated; “baseline” refers to transmigration without CXCL12 input. Left panel, Bar plot presents CXCL12-driven transmigration activity of CD34⁺CD38⁻sLeX⁺ (red bars) and CD34⁺CD38⁻ sLeX^{-/low} cells (white bars) cells. Right panel, bar plot presents transmigration activity of CD34⁺CD38⁺ sLeX⁺ (red bars) and CD34⁺CD8⁺ sLeX^{-/low} cells (white bars) cells. N=3, with replicates of 2 wells for each assessment. Data presented as mean±SD. Statistics: Paired t-test, *P<0.05, **P<0.01, ns indicates sample means not significantly different.

FIG. 3 shows that HSC_{conv}s express uniformly high levels of sLeX while more differentiated HSPCs contain distinct sLeX+ and sLeX⁻ subpopulations. (A) Representative contour plots showing binding of mAb HECA452 to each individual HSPC subset (as indicated at the top left corner of each plot). Numbers denote frequency of cells (%). (B) (left panel) Aggregate data presenting frequency of sLeX expressing cells within HSPC subsets. Data are presented as Mean±SD, N=13 independent UCB donors. Statistics: Repeated measures ANOVA (P<0.0001) with Tukey's multiple comparisons test comparing means of each column. Square brackets indicate populations compared, *P<0.05, **P<0.01, ***P<0.001, ****P<0.0001. #P<0.0002 compared to every other population. (Right panel) Mean fluorescence intensity of HECA-452 binding to HSC_{conv}, MLP, and GMP. Data are presented as mean±SD of measurements from 13 independent donors. Statistics: Repeated measures ANOVA (P<0.0001) with Tukey's multiple comparisons test comparing means of each subset with the mean of every other subset. *P<0.05, **P<0.01. Results are presented as mean±SD. (C) Flow cytometry dot plot of a representative marrow-derived CD34+ HSPC population stained with antibodies to CD38 (Y-axis) and to sLeX (X-axis). As shown, among human CD34+ HSPCs, HSCs are localized within the CD38−/sLeX^{high} region of the dot plot, whereas GMP are localized within the CD38+sLeX^{high} region and MEP are found within the CD38+sLeX^{low/-} region.

FIG. 4 shows sLeX expression within hematopoietic progenitor subsets identified by CD123 and CD45RA expression. (A) Gating strategy to identify CMP, GMP and MEP sunsets based on CD34, CD38, CD123, and CD45RA expression. (B) Left panel shows representative histograms of HECA-452 staining of cells comprising the CMP (Black histogram), GMP (Grey histogram) and MEP (Red histogram) subsets. Right panel shows summary of multiple experiments presenting sLeX density measured by mAb HECA452 binding (N=4 separate UCB/marrow donors). Statistics: Repeated measures ANOVA (P<0.005) with Tukey's multiple comparisons test; *P=0.014, **P=0.008.

FIG. 5 shows sLeX expression within subsets of MEPs as defined by MPL expression. (A) Gating strategy to identify megakaryocytic-committed cells, MEP-MPL+(CD34⁺CD38⁺CD135⁻CD45RA⁻CD110⁺), and erythroid-committed cells, MEP-MPL−(CD34⁺CD38⁺CD135⁻CD45RA⁻CD110⁻). (B) Left panel shows representative histograms showing HECA-452 binding to the two MEP subpopulations. Right panel shows summary of multiple experiments presenting sLeX expression as measured by mean fluorescence intensity (MFI) of mAb HECA452 staining. N=3 separate UCB donors. Statistics: ratio paired t-test; *P=0.02.

FIG. 6 shows sLeX expression and E-selectin binding of human HSPC subsets. (A) MFI of mAb CSLEX1 staining of HSPC subsets, Mean±SEM; N=3 independent UCB donors. Statistics, repeated measures ANOVA (P=0.0084), *P<0.05. (B) MFI of E-selectin binding (E-selectin-Ig chimera staining) of HSPC subsets, Mean±SEM; N=3 independent UCB donors.

FIG. 7 shows the analysis of expression of glycoproteins that display sLeX on human HSPC subsets. (A) Expression of sLeX-carrying glycoproteins on HSPC subsets. Mean fluorescence intensities of CD162 (PSGL-1, N=3-5) (top panel), CD44 (N=5) (middle panel), and CD43 (N=5) (bottom panel) among human HSPC subsets. Data are presented as mean±SD of 3-5 independent experiments. Statistics: Ordinary one-way ANOVA (P<0.0001 for top and middle panels and P=0.6745 for bottom panel) with Tukey's multiple comparison test. For top panel, Square bracket indicates comparison between two data points, and *P<0.05. For top and bottom panels, #P<0.004 compared to all other subsets. (B) Staining of untreated and pronase-treated CD34+ HSPCs with mAbs against PSGL-1, CD44, CD43, and sLeX (clones HECA452 and CSLEX). Top panels present representative flow cytometry histograms. Red lines indicate untreated cells, solid black lines indicate pronase-treated cells, and dotted line is respective isotype-control mAb staining. Bottom panels are aggregate data (n=3) measuring cell surface levels of PSGL-1, CD44, CD43, and sLeX (staining with anti-sLeX mAbs HECA452 and CSLEX1), on untreated and pronase-treated CD34⁺ human HSPCs. Statistics: Student's t-test, ****P<0.0001, ***P=0.0007.

FIG. 8 shows the various glycosyltransferases and glycosidases that regulate human sLeX expression. The first step in creation of sLeX requires the addition of GlcNAc to an acceptor glycan, which is then modified in § (1,4)-linkage by Gal via the action of the enzyme β4GALT1 (Step 2) to create a Type 2-LacNAc. (A) For initial creation of Type 2-LacNAc on a glycoprotein N-glycan, GlcNAc must first modify a mannosyl backbone, a reaction catalyzed by the enzyme MGAT1 (Stolfa et al., 2016). (B) On glycoprotein O-glycans, initial GlcNAc addition is catalyzed by the enzyme GCNT1 to create a “core-2” structure (Nonomura et al., 2004). (C) P4GALT1 catalyzes addition of the galactose (Gal) residue to GlcNAc to create Type 2-LacNAc; Type 2-LacNAc units form disaccharide polymers by sequential addition of GlcNAc and Gal (“polylactosamine chain”). Whether additional Type 2-LacNAc units are added or not, the penultimate step (Step 3) of sLeX assembly is the addition of NeuAc (sialic acid) to a terminal Type 2-LacNAc unit, a reaction programmed by three different members of the α(2,3)-sialyltransferase (α(2,3)-ST) family, ST3GAL3, ST3GAL4, or ST3GAL6 (Mondal et al., 2015; Yang et al., 2012). The terminal Type 2-α(2,3)-sialylLacNAc (NeuAcu(2,3)-Galβ(1,4)-GlcNAc-R) can then be catalytically converted to sLeX by various members of the α(1,3)-fucosyltransferase (α(1,3)-FUT) family. In humans, there are six α(1,3)-FUT isoenzymes (FUT3, FUT4, FUT5, FUT6, FUT7, and FUT9) (Mondal et al., 2018): four of these, viz, FUT3, FUT5, FUT6, and FUT7 efficiently add α(1,3)-Fuc to Type 2-α(2,3)-sialylLacNAc creating sLeX, while FUT4 and FUT9 principally add α(1,3)-Fuc to the “neutral” (i.e., unsialylated) Type 2-LacNAc unit, forming the trisaccharide known as “Lewis-X” (LeX, CD15, a known myeloid marker). Of these enzymes, there is variable potency: for creation of sLeX, FUT6 is the most potent (followed by FUT7); for creation of LeX, FUT9 is the most potent (Mondal et al., 2018). Notably, FUT3, FUT5, and FUT6 are multi-specific, as they can create both sLeX and LeX. Importantly, addition of α(1,3)-Fuc to Type 2-α(2,3)-sialylLacNAc is the terminal step in sLeX biosynthesis, as α(2,3)-sialyltransferases cannot sialylate an LeX acceptor to create sLeX; as such, fucosylation of Type 2-LacNAc to create LeX prior to the α(2,3)-sialylation of the Type 2-LacNAc (to create Type 2-α(2,3)-sialylLacNAc) blocks sLeX generation. Neuraminidases (NEU1 and NEU3), enzymes that remove sialic acid, and fucosidases (FUC1 and FUC2), enzymes that hydrolyse fucose linkages, also play important roles in controlling sLeX levels on the cell surface. Neuraminidases can cleave sialic acid residues and convert sLeX to LeX, and these enzymes can also desialylate Type 2-α(2,3)-sialylLacNAc to yield (unsialylated) Type 2-LacNAc. Fucosidases can remove the fucose residue from sLeX to engender core Type 2-α(2,3)-sialylLacNAc, or from LeX to engender backbone Type 2-LacNAc.

FIG. 9 shows transcriptomic Assessment in Human HSPC Subsets of Glycosyltransferase Genes Regulating sLeX Expression, and GAP Analysis of Type 2-α(2,3)-sialylLacNAc and Type 2-LacNAc Cell Surface Levels on Human HSPC Subsets. (A) Expression of glycosyltransferase genes involved in sLeX Expression measured by qRT-PCR. Top Panel, Expression of MGAT1, enzyme that initiates complex and hybrid N-glycan synthesis, GCNT1, enzyme initiates synthesis of core-2 O-glycans, and β4GALT1, galactosyltransferase that adds 0(1,4)-linked galactose residue to N-Acetylglucosamine creating Type 2 lactosamine, GDP-fucose transporter (SLC35C1) that controls levels of GDP-fucose within Golgi Apparatus, α_{L}-fucosidases (FUC1 and FUC2) that cleave α(1,3)-linked fucose. Middle panel, Expression of α(2,3)-sialyltransferases (ST3Gal3, ST3Gal4, and ST3Gal6) that add α(2,3)-sialic acid to Type 2 lactosamine, and neuraminidases (NEU1 and NEU3) that remove sialic acid residues from glycoproteins or glycolipids. Bottom panel, Expression levels of the six α(1,3)-fucosyltransferases, FUT3, FUT4, FUT5, FUT6, FUT7, and FUT9. Statistics: Ordinary one-way ANOVA (p-values are indicated in panel (B)), with multiple comparison test according to “Two-stage linear step-up procedure of Benjamini, Krieger and Yekutieli (Benjamini et al., 2006)” *FDR<0.05, ‡FDR<0.01, ‡FDR<0.001. FDR=False discovery rate. (C) GAP Analysis: Measurement of the levels of acceptor Type 2-α(2,3)-sialylLacNAc units on the surface of buffer-treated (BT; control) and α(1,3)-exofucosylated HSPC subsets (sLeX detected by HECA452 mAb staining; LeX detected by H198 mAb staining). Top panel & Middle Panels: Measurement of the levels of acceptor Type 2-α(2,3)-sialylLacNAc units. Top panel shows representative histograms of HECA452 mAb binding to either buffer-treated (BT, black empty histogram), or FT6-treated (FT6, red histogram) human HSPC subsets. Middle panel presents aggregate data for n=5 independent experiments of changes in sLeX expression between BT- and FT-treated subsets. Statistics: Paired t test, *P<0.05, ns indicates sample means not significantly different. Bottom panel: GAP Analysis of levels of neutral Type 2-LacNAc on human HSPC subsets. Representative histograms showing measurement of the levels of Le^{X} (HI98 binding) on the surface of buffer-treated (BT; control) and FT9-exofucosylated (FT9) HSPC subsets. Black empty histograms indicate buffer-treated cells and blue histograms indicate FT9-treated cells. (D) Graphic display of the percent of sLeX+ cells (cells stained using mAb CSLEX-1 (Y-axis)) among the different human HSPC subsets (X-axis) before and after α(1,3)-fucosylation of cell surface Type 2-α(2,3)-sialylLacNAc units using FT7 (n=3 separate donors). The glycosyltransferase FT7 is capable only of catalyzing the installation of fucose in α(1,3)-linkage to GlcNAc within a Type 2-α(2,3)-sialylLacNAc unit. Note that consistent with results using FT6 (as shown in (C) middle panel), the MPP1, MPP2, MLP, CMP, GMP, and MEP subsets all become uniformly sLeX+(at 100% level of sLeX-positivity) following FT7-mediated α(1,3)-fucosylation. The fact that α(1,3)-fucosylation of certain HSPC subsets (most notably MPP2s, CMPs, and MEPs) raises their cell surface sLeX expression to levels equivalent to that of HSCs indicates that these cells are thus robustly capable of engaging E-selectin, and, thereby, have become operationally specialized for osteotropism and engraftment.

FIG. 10 shows that exofucosylation accelerates myeloid engraftment by CD34⁺CD38⁺ HSPCs. (A) Schematic of xenotransplantation assay for evaluating short-term hematopoietic engraftment in NSG mice. Flow-sorted CD34⁺CD38⁺ human HSPCs (“HPCs”) were either treated with buffer alone (BT) or exofucosylated with FT6 (FT6) and injected into sub-lethally irradiated (225 cGy) NSG mice. Control mice received untreated CD34⁺CD38⁻ HSPCs (“P0”). Blood was collected from mice on indicated time points and analyzed using flow cytometry after co-staining with antibodies against mouse and human CD45. (B) Left panel, pre-injection CD34 and CD38 expression. Right panel, flow cytometry histograms of sLeX expression in BT (clear histogram) and FT6-treated (red histogram) cells. (C) Proportion of mice with human cell engraftment at various time points (left panel), and kinetics of human cell engraftment in NSG mice between 7 and 31 days post-transplantation (right panel). Engraftment steadily increased in mice receiving P0 cells; engraftment diminished within 31 days in those receiving HPCs, but with earlier recovery and more prominent engraftment in FT6-treated HPCs. N=10 mice in each treatment group. Statistics: Welch's paired t-test, **P=0.0017. (D) Schematic of experimental workflow to evaluate proliferation and differentiation potential of HSPCs. CD34⁺ HSPCs were isolated and divided into three treatment groups: untreated (UN), buffer treated (BT), and exofucosylated using FT6 (FT6). Cell proliferation was measured by staining with Ki67. Differentiation potential was measured using methyl cellulose colony forming unit (CFU) assay. (E) sLeX levels in UN, BT, and FT6 cells. N=4. Statistics: Repeated measures ANOVA with Tukey's multiple comparison test. **P<0.01, ns indicates no significant difference. (F) Expression of the proliferation marker Ki67 within CD34⁺CD38⁻ (left panel) and CD34⁺CD38⁺ (right panel) HSPC subpopulations. N=4. Statistics: Paired t-test, ns indicates not significant. (G) Colony forming efficiency of UN (white bar), BT (grey bar), and FT6-treated (red bar) cells. N=3. Statistics, Repeated measures ANOVA (P=0.8382). (H) Distribution of hematopoietic colonies formed by UN, BT, and FT6 cells. N=3, statistics, repeated measures ANOVA for each colony type.

FIG. 11 shows that the transcriptional profile of CD34⁺CD38⁻sLeX^{high} cells parallels that of HSC_{conv}s. (A) Gating strategy to isolate five UCB HSPC subpopulations, based on sLeX expression, for RNA sequencing analysis. CD34⁺UCB HSPCs (top left) were subdivided into (1) P1=CD34⁺CD38⁻sLeX^{high} (“sLeX^{high}” is highest 10% fraction of sLeX-staining MFI); (2) P2=CD34⁺sLeX^{high}; (3) P3=CD34⁺sLeX^{-/low} (“sLeX^{-/low}” is lowest 10% fraction of sLeX-staining MFI); (4) HSC_{conv}=CD34⁺CD38⁻CD90⁺CD45RA⁻CD49f⁺; and (5) HPC=CD34⁺CD38⁺. (B) Principal component analysis of the 459 genes differentially expressed across the five isolated HSPC subsets (based on DEseq2 differential expression analysis). Only the first two principal components are presented as they represent majority of variation in the dataset. (C) Pearson's correlation matrix representing relationships between individual subpopulations. The number of genes having significantly different expression (FDR<0.05, moderated t-test with Benjamini-Hochberg correction) between subset pairs are indicated within each box. (D) DAVID analysis indicating overrepresented categories between P1 and HPC subsets. (E) Heatmap presenting genes that regulate cell cycle in the isolated HSPC subsets. (F) Cluster dendrogram presenting population distances (Pearson's correlation-based distance). (G) Heatmap presenting hierarchical clusters of 459 differentially expressed genes. (H) Scree plot presenting proportions of total variance in the data explained by each principal component.

FIG. 12 shows results of xenotransplantation assays to measure long-term human engraftment in NSG mice. 500, 1000, or 2000 P1 (CD34⁺CD38⁻sLeX^{high}) P2 (CD34⁺sLeX^{high}), HSC_{conv}, and HPC (CD34+CD38+) cells were injected into sub-lethally (225 cGy) irradiated NSG mice. Blood was collected at 2, 4-, 6-, 12-, and 24-weeks post-transplantation, and interrogated using flow cytometry after staining with antibodies against mouse and human CD45. (A) Percentage of mice in each group displaying human cell engraftment at each time point. (B) Kinetics of human cell engraftment in mice receiving P1, P2, and HSC_{conv} populations between 2 and 24 weeks.

FIG. 13 shows that α(1,3)-exofucosylation of UCB HSPCs enhances bone marrow homing of the cells. (A) Schematic of xenotransplantaion assay for evaluating marrow homing of HSPCs. Buffer-treated (BT) and FT6-exofucosylated CD34⁺ UCB HSPCs were stained with CFSE and SNARF-1 respectively (dye staining was reversed for some experiments), mixed in 1:1 ratio, and injected into individual NSG (NOD-scid IL2Rgamma^{null}) mouse (8 mice total). Bone marrow was harvested 24 hours after injection and marrow cells were interrogated by flow cytometry for detection of CFSE and SNARF-1 labeled cells. (B) Representative histograms of sLeX display on BT (clear histogram) and FT6-exofucosylated (red histogram) cells. (C) Representative flow cytometry dot plots showing pre-injection mixture of CFSE and SNARF-1 labeled cells (Left panel), bone marrow of NSG mouse without any human cell injection (middle panel), and CFSE- and SNARF-1-positive cells within mouse bone marrow (right panel). (D) Aggregate data shows fold-difference in homed human cells in control mice (mice that received CFSE- and SNARF-1-labeled BT cells) and experimental mice that were injected with CFSE- and SNARF-1-labeled (or vice versa) BT and FT6-treated HSPCs (red box). N=8 mice in total (4 for each dye combination). Statistics: Mann-Whitney U test, P=0.0143 indicates statistically significant difference.

### DETAILED DESCRIPTION

### Cell Separation/Selection

According to some aspects, the present disclosure provides a method of selecting cells from a population of nucleated cells based on, inter alia, expression level of sLeX. As disclosed herein, it has been surprisingly discovered that sLeX expression alone or in combination with other cell markers can be used to identify and isolate various HSPC subsets/types that have myriad medical applications. In some embodiments, a population of nucleated cells can be sorted based on the level of sLeX expression, wherein sLeX^{high} cells comprise hematopoietic stem cells (HSCs) and/or granulomonocytic progenitors (GMPs), and sLeX^{low/-} cells comprise megakaryocyte-erythroid progenitors (MEPs) and/or endothelial progenitor cells (EPCs) and compositions comprising such selected cells. In some embodiments, the cells isolated as disclosed herein can be cultured ex vivo to produce therapeutically relevant numbers of cells. In some embodiments, the cells isolated as disclosed herein can be further contacted with an α(1,3)-fucosyltransferase (together with a tagged or untagged GDP-fucose) capable of creation of sLeX from an acceptor terminal α(2,3)-sialylated Type-2 lactosamine. In some such embodiments, specific cell types (such as MEPs and/or EPCs) are selected based on native sLeX expression and then enforced to have additional sLeX expression to enhance the isolated cells' ability to home to target sites (such as sites of inflammation and/or bone marrow).

According to some aspects, the present disclosure provides a method for selecting one or more lineage negative (lin⁻) hematopoietic stem/progenitor cells (HSPCs) from within a heterogenous population comprising the steps of: contacting the heterogenous population of lin⁻ HSPCs with a binding molecule for sialylated Lewis X (sLeX); measuring the amount of sLeX present on individual cells in the heterogenous population of lin⁻ HSPCs using the binding molecule; and selecting for one or more of sLeX^{high} cells based on the level of sLeX expression of the cells, wherein the sLeX^{high} cells are the cells having the highest 15% sLeX expression level within the heterogenous population of lin⁻ HSPCs. Alternatively, one procedure that may be used at the first stage to enrich lin−cells from a heterogenous population of hematopoietic cells is to (positively) select for cells expressing CD34, as CD34+ HSPCs are inherently enriched in lin⁻ HSPCs; selection for sLeX^{high} cells could then proceed from the CD34+ fraction, or could proceed after negative selection of CD38− cells within the CD34+ fraction (selection of sLeX^{high} cells within the CD34+CD38− fraction of HSPCs).

As used herein the terms “selecting,” “collecting,” “enriching,” “separating,” and “sorting” of cells refers to an operation that segregates cells into groups according to a specified criterion (including but not limited to, differential staining and marker expression) as would be known to a person skilled in the art such as, for example, sorting using FACS. Any number of methods to differentiate the specified criterion may be used, including, but not limited to the use of anti-marker antibodies together with a wide variety of reporter fluorochrome dyes, either as using direct mAb-fluorochrome conjugates or in a two-step process by which molecules (e.g., anti-Ab secondary antibodies, protein G, etc.) that are conjugated to fluorochromes can be used to stain cells that bear (unconjugated) anti-marker mAbs. Flow cytometry of fluorochrome-stained cells allows for quantification of the expression level of a given marker on discrete cells, and is the predominant technique for subsequent enrichment of cells on the basis of marker expression levels (e.g., useful for detection and subsequent flow-sorting of the sLeX^{high} HSPC fraction of cells). Bulk separation of cells can be accomplished conveniently by various techniques, including, but not limited, use of antibodies conjugated to magnetic beads (with subsequent cell collection using magnets), antibodies tagged with biotin (with subsequent collection of cells via avidin- or streptavidin-coated support surfaces), antibodies affixed to solid matrices (e.g., “panning”), or passing cells through a column in which antibodies are fixed to beads or other matrices. For binding to sLeX, there are numerous commercially available mAbs that can be used (e.g., HECA452, CSLEX-1, etc.) and E-selectin-Ig chimera is also commercially available. E-selectin-Ig chimera functions essentially as an anti-sLeX mAb, wherein binding to sLeX is calcium dependent, and, as such, this reagent has the advantage of releasing its binding to sLeX when exposed to EDTA (thereby fully freeing sLeX display).

As used herein, the “selecting,” “collecting,” “enriching,” “separating,” and “sorting” of cells may comprise a positive or negative selection step. A “positive” selection used in reference to cell surface markers means making a selection for the presence of a certain cell marker by directly selecting for cells expressing the desired characteristic marker. The term “negative” selection as used in reference to cell selection generally involves enriching a pertinent cell population from within a heterogenous population of cells indirectly, i.e., by removing a pertinent cell subpopulation (from within the heterogenous cell mixture) that does not contain the phenotypic and/or biologic characteristic that one seeks to target: for example, removing CD38⁺ cells from a heterogenous population of HSPCs “negatively” selects for CD38− cells. This said, importantly, one can also negatively enrich for a subpopulation of cells by targeting a phenotypic and/or biologic characteristic that is not co-expressed by the population of cells that one wishes to select: e.g., CD34⁺ cells can be enriched from a heterogenous population of HSPCs by removal of lin+ cells (and, more fundamentally, differential centrifugation or gradient-based centrifugation can serve to remove undesired cells (and can also be used to positively enrich for desired cells), and flow cytometry-based forward scatter and side scatter characteristics of cells enables selective gating to exclude cell subpopulation(s) that one does not wish to obtain (as well as to positively select for a given subpopulation via a specific side scatter/forward scatter pattern). Moreover, “negative” selection does not necessarily imply that the cells that are removed from a given heterogenous cell population lack expression of a given marker that is present on the cells which one seeks to select: e.g., using FACs, negative selection can be applied to enrich a target population by sort/removing cells that have a certain level of expression for the pertinent cell marker, and, as such, the term “negative selection” does not imply that the selected cells lack expression of the certain marker (for example, an sLeX^{high} fraction of HSPCs can be enriched by negative selection by removing those HSPCs that express sLeX at mean channel fluorescence intensity (MFI) staining levels that fall below the “high” MFI cut-off level). Thus, a method employing positive or negative selection of cells bearing a pertinent phenotypic marker can be used to select cells that express that same marker, and a positive or negative selection of cells bearing a given marker and/or a given set of markers can also be used to select cells that do not express a particular marker.

As used herein, “sLeX^{high}” cells refers to cells that express sialylated Lewis X (sLeX) at a level that is greater than at least 85% of the cells that express sLeX within that population of HSPCs; i.e., the cells harbor sLeX surface density above the 85th percentile of the sLeX surface density (expression level) within the cells of the population. Thus, sLeX^{high} cells include the cells expressing sLeX at an amount that is greater than 90%, 95%, or even 99% of the cells that express sLeX in a population.

The term “binding molecule” as used herein refers to any molecule that binds with specificity to a particular target (such as a cell marker). Examples of binding molecules include, but are not limited to, classes such as antibodies, peptides, chimeric constructs containing a binding molecule linked to a non-binding moiety, small molecules, nucleic acids and the like. As used herein the term “antibody” or “antibodies” means a polypeptide that specifically binds and recognizes an analyte (antigen) such as CD34, CD38, or sLeX, or a specific antigenic determinant (epitope) thereof. The term “antibody” or “antibodies” is used herein in the broadest sense and encompasses various antibody structures, including but not limited to monoclonal antibodies, polyclonal antibodies, multispecific antibodies (e.g., bispecific antibodies), and antibody fragments, so long as they exhibit the desired antigen/epitope-binding activity. Non-limiting examples of antibodies include, for example, intact immunoglobulins and variants and fragments thereof known in the art that retain binding affinity for the antigen. Examples of antibody fragments include but are not limited to Fv, Fab, Fab′, Fab′-SH, F(ab′)₂; diabodies; linear antibodies; single-chain antibody molecules (e.g. scFv); and multispecific antibodies formed from antibody fragments. Antibody fragments include antigen binding fragments either produced by the modification of whole antibodies or those synthesized de novo using recombinant DNA methodologies (see, e.g., Kontermann and Dubel (Ed), Antibody Engineering, Vols. 1-2, 2^{nd} Ed., Springer Press, 2010). Other examples of binding molecules include those that are effective to bind to cell surface glycans, including but not limited to E-selectin. Notably, among the selectins, though E-selectin prototypically binds sLeX with highest affinity, both L-selectin and P-selectin (and constructs containing the respective lectin domains of each of the selectins) can bind sLeX and could thus be used to stain (and select) for cells expressing sLeX.

In some embodiments, such binding molecules may be used to probe for a marker (to quantify abundance of the marker) or to select for cells having a particular marker. In some embodiments, such binding molecules may be used with one or more of the following for selection of cells with a particular marker: magnetic bead-tagged anti-determinant molecules (e.g., magnetic bead-conjugated anti-sLeX antibody, magnetic bead-conjugated E-selectin-Ig chimera), biotin-tagged anti-determinant molecule (e.g., biotin-tagged anti-sLeX antibody, biotin-tagged E-selectin-Ig chimera, with subsequent collection using avidin- or streptavidin-conjugated beads/matrices), fluorescence-activated cell sorting (FACS) utilizing fluorochrome-tagged anti-determinant molecule (e.g., direct (one-step) using a fluorochrome-tagged anti-determinant molecule, or indirect (two-step) using a fluorochome-tagged secondary reagent that recognizes the (primary) anti-determinant molecule (indirect (two-step) fluorochrome labelling of the cell)), chemically-modified anti-determinant molecule (e.g., anti-determinant molecule modified to contain a “clickable” chemical reagent such as an alkyne or azide modification), “panning” by affixing the anti-determinant molecule to a solid support matrix, passage of cells over affinity columns containing the anti-determinant molecule attached to beads or other matrices, or other techniques providing accurate cell separation.

In some embodiments, a glycosyltransferase is used to install a chemically reactive group, orthogonal functional group, or molecular tag for the selection. For example, in some embodiments, addition of a donor GDP-fucose wherein the fucose has been modified by methods known in the art with a chemically reactive group, or orthogonal functional group, or molecular tag (e.g., biotinylated GDP-fucose, azido-GDP-fucose, etc.) thereby allowing for subsequent linkage of other molecules onto the installed fucose within cell surface lactosaminyl glycans (examples of this approach include, but are not limited to, use of biotinylated GDP-fucose with subsequent complexing using streptavidin-conjugated molecules (see, e.g., Elhalabi and Rice, Current Medicinal Chemistry, vol. 6, No. 2, 108-109, 1999) and/or use of “click chemistry” (e.g., wherein an azido-containing fucose molecule is then complexed to an alkyne-containing molecule). (see, e.g., Sekhon, BS, Click chemistry: Current developments and applications in drug discovery, J. Pharm. Educ. Res. Vol. 3, Issue 1, 2012; e.g. Click-IT Fucose Alkyne, Thermo Fisher, At. No. C10264) In other embodiments, molecules covalently linked to the donor nucleotide fucose (i.e., GDP-fucose with covalent attachment of additional molecule(s)) can be stereospecifically added to a given cell surface by use of fucosyltransferases, thereby rendering a distinct molecular signature onto cell surface lactosaminyl glycans that can thus provide the ability to select the pertinent cell using ligands that bind to the relevant molecular moiety.

As used herein “lineage” or “lin” markers refer to markers that are used for detection of lineage commitment. Cells and fractions thereof that are negative for these lineage markers are referred to as “lin-”. In some embodiments, methods are used to deplete the “lin*” cells, such as by FACS. Human blood cell lineage markers are multiple, including (but not limited to): CD13 and CD33 for myeloid series cells; CD71 and glycophorin A (glyA; CD235) for erythroid series cells; CD41A and CD61 for megakaryocytic series cells (and platelets); CD10/CD19/CD20 markers for B cells; CD2/CD3/CD4/CD5/CD8 markers for T cells; CD16 and CD56 for NK cells; CD14, CD11b, and CD16 markers for monocytes; and CD1c, CD11c, CD303, and CD304 markers for dendritic cells.

In some embodiments, the methods disclosed herein comprise the steps of providing a population of cells that are CD34+. In some embodiments, the CD34+ cells are positively selected by specifically targeting the CD34 with a binding molecule. In some embodiments, the CD34+ cells are negatively selected by removing cells that are not CD34+. For example, in some embodiments, cells are selected for being lin−(by removing the lin+ cells). The lin− HSPC population contains cells that are CD34+ and, therefore, a negative selection process for lineage markers can be used to select for CD34+ cells. However, the selection of the lin− cell population for enriching primitive (immature) HSPCs not only comprises CD34+ HSPCs, but also includes a very rare population of CD34− cells that also have the multipotency and self-renewal properties that define HSCs. Thus, HSCs are operationally found in lin− HSPC populations that harbor CD34+CD38− cells as well as CD34−/CD38− cells.

In some embodiments, the methods disclosed herein comprise the steps of selecting lin− HSPCs (comprising CD34+ cells) that are both CD38− and sLeX^{high}. As disclosed herein, such cells comprise hematopoietic stem cells. As used herein “hematopoietic stem cells” or “HSCs” refers to a cell of any origin (such as bone marrow, embryonic yolk sac, fetal or post-natal liver, fetal or post-natal spleen, blood (whether consisting of fetal blood, or umbilical cord blood, or post-natal blood (whether native or specifically enriched with HSPCs via “mobilization” (e.g., by administration to patients of cytokines such as G-CSF, or administration of inhibitors of CXCR4 such as plerixafor)) that is capable of long-term self-renewal and is multipotent in its capability to develop into all mature blood cell types (and platelets). Previously, selection of human HSCs on the basis of cell surface molecules required use of a pentad of markers: CD34⁺CD38⁻CD90⁺CD45RA⁻CD49f⁺. It has been surprisingly discovered that that high sLeX expression is characteristic of human HSCs, and, more specifically, that, among human CD34+CD38− HSCs, the marker combination consisting of CD90+, CD45RA−, and CD49f+ can altogether by supplanted by high sLeX expression. As such, human HSCs can be defined as lin−/CD38−/sLeX^{high} cells and/or as CD34+CD38−sLeX^{high} cells. As compared to the conventional pentad combination of markers, the use of a triad of markers greatly simplifies both the selection and separation of the human HSC. Importantly, the ability to condense the marker combination from 5 markers to 3 markers greatly improves both the efficiency and the yield of collection of HSCs: for any cell selection method and/or process, there is a significant loss of cells (e.g., due to issues related to the sensitivity and/or specificity of the method at each isolation step, cell damage, cell death, etc.) within both the starting cell population and the intended target cells (i.e., the predetermined, sought subpopulation) during each round of marker selection.

In some embodiments, the methods disclosed herein comprise selecting lin-HSPCs (comprising CD34+ cells) that are both CD38+ and sLeX^{high}. As disclosed herein, such cells comprise granulocyte/monocyte (granulomonocytic) progenitors (GMPs), which, as the name implies, is an oligoclonal hematopoietic progenitor population capable of producing monocytes and the various types of granulocytes (neutrophils, eosinophils, and basophils). Previously, GMPs were most often identified using the following marker combination: CD34+CD38−CD45RA+CD135+(or, CD123+ can be used interchangeably with CD135+). It has been surprisingly discovered that GMPs can be selected in lin−/CD34+ HSPCs by selecting for CD38⁺ and sLeX^{high} cells, which greatly simplifies the selection criteria for these cells.

According to some aspects, the present disclosure provides a method for selecting one or more human hematopoietic stem/progenitor cells (HSPCs) from within a heterogenous population of lin⁻ HSPCs comprising: contacting the heterogenous population of lin⁻ HSPCs with a binding molecule for sialylated Lewis X (sLeX); measuring the amount of sLeX present on individual cells in the heterogenous population of lin⁻ HSPCs using the binding molecule (e.g., using flow cytometry to quantify surface sLeX levels); and then selecting (e.g., via FACS) for one or more of sLeX^{low/-} cells based on the level of sLeX expression of the cells, wherein the sLeX^{low/-} cells represent the fraction of 15% within the heterogenous population of lin⁻ HSPCs that comprise cells having absent-to-lowest sLeX expression level (by FACS, this would be measured as the fraction of 15% of the population having the lowest fluorescence expression level of sLeX staining).

As used herein, depending on the composition of the heterogenous cell population from which the “sLeX^{low/-}” cells are selected, the collected cells may range in composition from a population of cells that are completely devoid of sLeX expression, or a population that may have a mixture of cells that lack sLeX and some that have low sLeX levels, or may be a population in which all cells express sLeX but at a relatively low level. This potential range of sLeX expression on the particular collected population of sLeX^{low/-} cells is predicated by the cut-off being set at selecting the 15% fraction of the target cell population that expresses the lowest levels of sLeX expression. Thus, it is anticipated that the collected sLeX^{low/-} population will commonly have at least some cells that lack sLeX expression.

In some embodiments, the methods disclosed herein comprise the steps of selecting lin− HSPCs (comprising CD34+ cells) that are both CD38− and sLeX^{low/-} cells. As disclosed herein, such cells comprise endothelial progenitor cells. As used herein “endothelial progenitor cells” or “EPCs” refers to cells that can initiate vasculogenesis and differentiate into endothelial cells. Previously, EPCs were identified by the following markers: CD34+ VEGFR2(KDR)+CD38−CD45−. It has been surprisingly discovered that EPCs can be selected from within lin⁻ HSPCs by selecting for CD38− and sLeX^{low/-} cells, which greatly simplifies the selection criteria for these cells.

In some embodiments, the methods disclosed herein comprise selecting lin-HSPCs (comprising CD34+ cells) that are both CD38+ and sLeX^{low/-}. As disclosed herein, such cells comprise megakaryocyte-erythroid progenitors (MEPs). As used herein, “megakaryocyte-erythroid progenitors” or “MEPs” refer to an oligoclonal progenitor subset that yields precursors of megakaryocytes (which make platelets) and erythrocytes (red cells). Previously MEPs could only be identified by the following markers: CD34+CD38+CD135−(or CD123−)CD45RA⁻. It has been surprisingly discovered that MEPs can be selected in lin-/CD34+ HSPCs by selecting for CD38+ and sLeX^{low/-} cells, which greatly simplifies the selection criteria for these cells.

In some embodiments, the heterogenous population of lin⁻ HSPCs is from bone marrow, umbilical cord blood, adult (post-natal) blood, fetal blood, fetal liver, fetal spleen, embryonic yolk sac, embryonic ventral endothelium of dorsal aorta, adult (post-natal) liver, or adult (post-natal) spleen.

In some embodiment, as an alternative to, or in addition to, selection for lin-HSPCs, CD34+ HSPCs are selected by one or more steps of positive selection (i.e., selecting for cells expressing CD34) or by negative selection (e.g., enriching for CD34+ cells by depleting a lineage+ nucleated cell population obtained from any of the stated sources of hematopoietic nucleated cells expressing lineage markers). Such cells may be used in the methods disclosed herein.

In some embodiments, the isolated cells as disclosed herein are characterized by both the presence of certain markers associated with specific epitopic sites (e.g., as identified by antibodies) and the absence of certain markers (e.g., as identified by the lack of binding of certain antibodies). As disclosed herein, it is not necessary to select for a marker specific for stem cells. By using a combination of negative selection (removal of cells) and positive selection (isolation of cells), a substantially homogeneous stem cell composition can be achieved.

In some embodiments, cell selection can begin with an initial “crude” separation. For example, the source of the cells may be the bone marrow (fetal, neonate or adult) or other hematopoietic cell source (e.g., fetal or adult liver, spleen, peripheral blood, umbilical cord blood, and the like). In some embodiments, bulk separation (by methods such as use of magnetic beads, cell rosetting-based separation (as commercially available from StemCell Technologies), panning, etc.) may be used initially to remove large numbers of lineage-committed cells, namely major cell populations of the hematopoietic systems, including such lineages as erythrocytes (red cells), myelomonocytic cells (granulocytes and monocytes), and lymphocyte populations (T-cells, B-cells, and NK cells), as well as platelets. In some embodiments, at least 70% of the total hematopoietic cells will be removed. In some embodiments, it is not essential to remove every dedicated cell class, particularly the minor population members at the initial stage. In some embodiments, the platelets and erythrocytes will be removed prior to sorting using gradient centrifugation or other means. In some embodiments, positive selection to select a desired cell type will be used without prior removal of lin+ cells. In some embodiments there can be use of negative selection to enrich lin−cells with or without further rounds of negative selection to enrich for a subpopulation containing the desired cell type(s), so that in subsequent positive selection step(s), the number of non-target (contaminating) cells present within the remaining heterogenous cell population is minimized.

In some embodiments, it may be necessary to isolate a rare HSPC subset whose concentration within a given hematopoietic tissue may vary, in which case the source of the starting population of heterogenous hematopoietic cells will be critical. In some embodiments, bone marrow cells may be obtained from a defined anatomic source of bone, e.g., iliac crests, tibia, femora, vertebrae, calvarium, sternum, or other bone cavities, or other sources of HSPCs can be used from particular sources such as embryonic yolk sac, fetal and adult liver, fetal and adult spleen, and blood (including adult peripheral blood or umbilical cord blood or “mobilized” blood (e.g., cytokine-mobilized or plerixafor-mobilized). For isolation of bone marrow from fetal bone or other bone source, an appropriate isotonic solution may be used to flush the bone, such as a balanced salt solution supplemented with anti-coagulants (and, generally, free of divalent cations that could trigger the coagulation system), with or without serum or protein supplementation, without or with an acceptable buffer at low concentration (generally from about 5-25 mM). In some embodiments, buffers include Hepes, phosphate buffers, lactate buffers, a Good's buffer such as a HEPES buffer, a 2-Morpholinoethanesulfonic acid (MES) buffer, etc. Suitable physiologically acceptable solutions include, for example, Hank's Balanced Salt Solution (HBSS), Dulbecco's Modified Eagle Medium (DMEM), or phosphate buffered saline (PBS).

In some embodiments, bone marrow may be aspirated from the bone in accordance with conventional techniques and collected in syringes containing anticoagulant-supplemented physiologic solutions. Hematopoietic cells can be extracted from tissues (e.g., spleen) that are minced then passaged over meshing to disrupt tissue integrity and release cells.

As disclosed herein, various techniques may be used to separate and/or isolate human HSPCs of interest. Any technique that is used should maximize the retention of cell viability. In some embodiments, fluorochrome-conjugated mAbs are used for identifying markers (surface membrane molecules) associated with particular cell lineages and/or stages of differentiation, and this approach is ideal for multi-parameter (multi-color) FACS sorting of the pertinent cell subsets delineated by mAb staining. The particular technique used will depend upon efficiency of separation, cytotoxicity of the methodology, ease and speed of performance, and necessity for sophisticated equipment and/or technical skill.

In some embodiments, “bulk” procedures for separation may involve differential centrifugation, gradient centrifugation, magnetic separation (using antibody-coated magnetic beads with subsequent cell collection via magnets), affinity chromatography (including, but not limited, lectin-based affinity chromatography to enrich for cells bearing target glycan structures, use of antibodies affixed to beads, or use of antibodies conjugated to biotin with subsequent separation via binding to avidin or streptavidin, etc.), cytotoxic agents joined to a monoclonal antibody or used in conjunction with a monoclonal antibody (e.g., complement and/or cytotoxins), and “panning” with antibody attached to a solid matrix (e.g., plate), or other convenient techniques. As shown in Supplemental Table 1, in our experience the percentage of operationally/developmentally-distinct human HSPC subsets within a population of CD34− expressing HSPCs obtained from human umbilical cord blood (UCB) (i.e., within the CD34+ HSPC population in UCB) varies widely dependent on the relevant subset, from as low as <2% (e.g., HSCs) to as high as >15% (e.g., CMPs) of the CD34+ cells. The use of such “bulk” techniques would enrich many-fold (ideally, at least 3-10-fold) the percentage of the desired cell subset within any heterogenous mixture of hematopoietic cells, with even higher-fold enrichment achievable depending on potential use of combinations of bulk techniques, in each case dependent on the specificity and sensitivity of the technique employed or isolation of cells bearing a pertinent marker. In most cases, bulk separation procedures would need to be supplemented with more fine separation techniques to ensure purity of the selected population(s) of interest: following the substantial enrichment of cells (e.g., for cells that are CD34+/CD38−), the cells of interest could then be further isolated/selected using methods with high specificity that are capable of discriminating the full range of expression of a given marker on a pertinent cell type of interest. In some embodiments, particularly in applications requiring highly accurate cell separation, especially of extremely rare cells (e.g., HSCs) or in the enrichment of cells that express variable levels (e.g., “high” or “low”) of a given marker (such as sLeX), the method of FACS serves as both a highly sensitive and highly specific technique, which can be used in tandem following bulk isolation (or, in itself, can achieve intended cell isolation using mAbs to different markers all at one time), and which can employ varying parameters to isolate a given cell type, e.g., a plurality of fluorescence color options and channels based on the combination(s) of lasers and fluorochromes chosen, low angle and obtuse light scattering detecting channels, impedance channels, etc. In some embodiments, human HSCs may be isolated by bulk separation combined with FACS to select a population of CD34+CD38−sLeX^{high} cells. The derived human CD34+CD38−sLeX^{high} population is expected to have a composition of HSCs in excess of 90%. In some embodiments, HSC isolation could encompass the sLeX^{high} fraction of any population of putative HSCs defined by pertinent markers (including, but not limited to groupings of the markers CD34+, CD38−. CD133+, EPCR+, CD90+, CD45RA−, CD49f+, GPRC5C+, or any other relevant HSC-associated marker recognized at present or in the future) or biologic/physical characteristics (e.g., “Rhodamine¹²³_{low} staining with low forward and low side scatter” characteristics by flow cytometry): i.e., this disclosure includes a human HSC population containing any permutation of phenotypic markers and/or biophysical properties that either singly or that could group together to identify a human HSC, in each case further used in combination with sLeX staining to isolate those HSCs based on the marker “sLeX^{high}”.

In some embodiments, after bulk separation to achieve substantial enrichment of desired HSPCs from a heterogenous mixture of hematopoietic cells, multicolor FACS could then be employed to isolate HSPC subsets of interest with high specificity and efficiency using relevant mAbs directed against cell markers of interest, in each case having the respective mAbs conjugated to a different fluorochrome. Fluorochromes which may find use in a multi-color analysis include phycobiliproteins, e.g., phycoerythrin and allophycocyanins, fluorescein, Texas red, etc. While each of the lineages may be separated in a separate step, desirably the lineages would be separated at the same time.

In some embodiments, the cells may be selected against dead cells, by employing dyes associated with dead cells (e.g., propidium iodide, LDS). In some embodiments, the cells are collected in a medium comprising serum (e.g., 2% fetal calf serum). In some embodiments, other techniques for positive selection may be employed, such as affinity columns, and the like.

As disclosed herein, the particular order of separation is not critical. In some embodiments, cells are initially separated by a coarse (bulk) separation such as negative selection for more primitive hematopoietic cells by use of antibodies against markers associated with lineage-committed cells (and/or, as indicated, against CD38), followed by a fine separation using positive selection by way of mAbs directed to markers associated with more primitive HSPCs.

In some embodiments, one or more cells from a population of heterogenous cells are selected by providing a selectin polypeptide, e.g., E-selectin (or P- or L-selectin). The advantage of these reagents is that they are calcium-dependent lectins; as such they affix to sLeX in presence of calcium, but then readily release the sLeX determinant upon calcium-chelation (e.g., by use of EDTA). Chimeric selectin-immunoglobulin heavy chain constructs are commercially available for each of the selectins, and these reagents essentially function as mAbs in detecting sLeX (with E-Ig having the greatest affinity for sLeX). The selectin-Ig constructs could be used in FACS, or immobilized on a solid phase to perform bulk collection of sLeX-bearing cells (or negative enrichment of sLeX−cells). Selectin-Ig molecules affixed to solid surfaces could also be used to select cells under defined hemodynamic shear conditions, wherein a heterogenous suspension of HSPCs could be passaged over the selectin-containing solid support under pertinent fluid flow conditions to achieve desired shear stress. Microscopy could be employed in real-time to allow the assessment of adherence of HSPCs to the selectin-coated surface, with subsequent release of bound cells by calcium-chelation. By varying the site density of affixed selectins and the fluid shear conditions, defined subsets of HSPCs (such as HSCs) would be enriched via binding based on their level of sLeX expression.

In some embodiments, methods of isolating a human HSPC (e.g., a self-renewing, multipotent HSC) from a population of human hematopoietic cells comprises contacting the cell population with one or more agents that specifically bind to sLeX under conditions sufficient to form a complex between the agents and sLeX. Complex formation is detected and the pertinent cells are then selected from the cell population thereby isolating the sLeX expressing cell from the cell population. Additionally, the cell/agent complex can be disrupted.

In some embodiments, compositions having greater than 90% human HSPCs that are sLeX^{high}, CD34+, and CD38− is achieved. The isolated cells are capable of long-term self-renewal
and long-term development of oligopotent HSPCs that then can create all pertinent blood cell types (and platelets). In some embodiments, a single HSC as defined as being sLeX^{high}, CD34+, and CD38− could suffice to reconstitute hematopoiesis in a host mammal, including a human.

In some embodiments, the selected HSPC subsets are capable of being propagated and expanded in vitro. As used herein, “expand”, “expanding,” “expansion” and like terms means to increase the number of cells (such as stem or progenitor cells) in the population relative to the number of cells in the original population using any of the methods known to those skilled in the art. The expansion could occur in the presence or absence of feeder cells. In some embodiments, the expansion is at least 40-fold compared to the original number of HSPCs in the population. In some embodiments, the expansion is at least 20-fold, 100-fold, 150-fold, 200-fold, 250-fold, or 500-fold compared to the original number of HSPCs. In some embodiments, the HSPC subsets may be generated ex vivo from embryonic stem cells or induced-pluripotent stem cell populations. In some embodiments, the selected cell can be expanded while remaining substantially undifferentiated. As used herein, a cell population is “substantially undifferentiated” if a sufficient number of cells in that population retain the ability to self-renew (at least for some period of time, with unlimited renewal being a property of the HSC) and can give rise to various differentiated cell types when transplanted into a recipient (e.g., in the case of an HSC population, repopulating the entire hematopoietic lineage when transplanted into a host). As used herein, “without significant differentiation” means the expanded HSPC population has a sufficient number of cells that maintain a multi-lineage differentiation potential such that the full scope of pertinent lineage development for the relevant subset may be regenerated upon transplantation of the expanded HSPC population into a recipient. Thus, in the case of an HSC population, the expanded HSC population, when transplanted into a recipient, would be capable of regenerating the entire hematopoietic cell lineage, whereas MEPs would be expected to generate platelets and erythrocytes.

### Methods of Identifying Cells Containing Unfucosylated Type 2-Lactosaminyl Glycans

According to some aspects, the present disclosure provides various methods of identifying and/or isolating cells that express pertinent precursor Type 2-lactosaminyl glycans. It has been surprisingly discovered that this new analytic technique (termed “Glycosyltranserase Acceptor-Product Analysis (GAP)”) is both specific and quantitative, and may be used to identify and sort a population of nucleated cells. This method comprises contacting cells with one or more α(1,3)-fucosyltranserases together with GDP-fucose to identify subsets of cells that can accept the fucose substitution as demonstrated by consequent increased expression of either sLeX or LeX. As disclosed herein, the high level of specificity of GAP analysis is based on the high specificity of the glycosyltranserases for their pertinent acceptor glycans. This entirely new approach obviates the need for high specificity reagents to detect the reaction product, because the specificity of the catalytic reaction provides the requisite specificity for detection of the acceptor (precursor) glycan. Quantification (such as flow cytometry quantification) of target (product) glycan expression is done before the glycosyltransferase reaction (to determine a baseline) and after the glycosyltransferase reaction (to determine the level of enforced expression). As disclosed herein, it has been surprisingly discovered that the surface of all HPSCs contain an equivalent total amount α(2,3)-sialylated LacNAc units, the only difference being that certain HSPC subsets α(1,3)-fucosylate the α(2,3)-sialylated LacNAc units more than others. GAP analysis is the only technique that is capable of providing this type of information about a population of cells.

GAP analysis is a method for grading the level of expression of terminal α(2,3)-sialylated Type-2 lactosamine units on cells comprising the steps of: (a) measuring the baseline level of expression of sialylated Lewis X (sLeX) on the surface of one or more cells; contacting the one or more cells with an α (1,3)-fucosyltransferase capable of creation of sLeX from an acceptor terminal α (2,3)-sialylated Type-2 lactosamine together with a nucleotide donor sugar (GDP-fucose); (b) measuring the level of sLeX expression following α(1,3)-fucosylation reaction, wherein the increase in sLeX expression from Step (a) to that following Step (b) indicates the level of “free” (i.e., unfucosylated) terminal α(2,3)-sialylated Type-2 lactosamine units on the surface of the cell(s).

According to some aspects, the present disclosure provides a method for grading the level of expression of terminal “neutral” Type-2 lactosamine units on cells comprising the steps of (a) measuring the level of expression of Lewis X (LeX) on the surface of one or more cells; contacting the one or more cells with an α(1,3)-fucosyltransferase capable of creating LeX from an acceptor terminal “neutral” Type-2 lactosamine together with a nucleotide donor sugar (GDP-fucose); (b) measuring the level of LeX expression on the fucosyltransferase-treated cells, wherein the increase in LeX level expression from Step (a) to that following Step (b) indicates the level of terminal “neutral” Type-2 lactosamine units on the cell(s).

In some embodiments, the measuring of step (a) and step (b) comprises contacting the sLeX or LeX with a fluorescent binder and measuring mean fluorescence intensity (MFI) by flow cytometry. As used herein, “mean fluorescence intensity” or “MFI” is a measure of the density (abundance) of a specific epitope/antigen/marker on the surface of a cell as quantified by the level of fluorescence emitted (brightness) by that cell.

In some embodiments, for the generation of sLeX, the α(1,3)-fucosyltransferase is selected from the group consisting of FTVI, FTVII, FTIII, FTV, or FTIV as defined herein. In some embodiments, the fucose of the GDP-fucose is modified with a selection tag that allows for separation and/or isolation of those cells containing the installed fucose bearing the selection tag.

In some embodiments, for the generation of LeX, the α(1,3)-fucosyltransferase is selected from the group consisting of FTIX, FTVI, FTIV, FTIII, or FTV as defined herein. In some embodiments, the fucose of the GDP-fucose is modified with a selection tag that allows for separation and/or isolation of those cells containing the installed fucose bearing the selection tag.

In some embodiments, the fucose of the GDP-fucose has been modified by methods known in the art with a chemically reactive group, or orthogonal functional group, or molecular tag (e.g., biotinylated GDP-fucose, azido-GDP-fucose, etc.) thereby allowing for subsequent linkage of other molecules onto the installed fucose present within cell surface lactosaminyl glycans (examples of this approach include, but are not limited to, use of biotinylated GDP-fucose with subsequent complexing using streptavidin-conjugated molecules (see, e.g., Elhalabi and Rice, Current Medicinal Chemistry, vol. 6, No. 2, 108-109, 1999) and/or use of “click chemistry” (e.g., wherein an azido-containing fucose molecule is then complexed to an alkyne-containing molecule). (see, e.g., Sekhon, BS, Click chemistry: Current developments and applications in drug discovery, J. Pharm. Educ. Res. Vol. 3, Issue 1, 2012; e.g. Click-IT Fucose Alkyne, Thermo Fisher, At. No. C10264). In other embodiments, molecules covalently linked to the fucose (i.e., GDP-fucose with covalent attachment of additional molecule(s)) can be stereospecifically added to a given cell surface by use of fucosyltransferases, thereby rendering a distinct molecular signature onto cell surface lactosaminyl glycans that can thus provide the ability to select the pertinent cell using ligands that bind to the relevant molecular moiety. In some embodiments, the selection tag consists of a chemically “tagged” GDP-fucose covalently modified with a fluorochrome, a clickable chemical group, biotin, a radiolabel, or any other molecule covalently linked to the fucose moiety within GDP-fucose that can be used to identify the installed fucose, as disclosed herein.

### Exofucosylation

According to some embodiments, cells of the present disclosure are contacted with a glycosyltransferase to enforce a glycan on the cell surface. In some embodiments, the glycosylstranferase is a human glycosyltransferase. In some embodiments, the glycosylstranferase is a non-human glycosyltransferase. According to some embodiments, fucosylated lactosaminyl glycans are enforced by a member of the α(1,3)-fucosyltransferase family. The human α(1,3)-fucosyltransferase family includes Fucosyltransferase III (also called FTIII, FT3, FUTIII, or FUT3), Fucosyltransferase IV (also called FTIV, FT4, FUTIV, or FUT4), Fucosyltransferase V (also called FTV, FT5, FUTV, or FUT5), Fucosyltransferase VI (also called FTVI, FT6, FUTVI, or FUT6), Fucosyltransferase VII (also called FTVII, FT7, FUTVII, or FUT7), Fucosyltransferase IX (also called FTIX, FT9, FUTIX, or FUT9), and variants thereof. The cDNA/protein sequences for the α(1,3)-fucosyltransferase family are as follows

| Name | GenBank Acc. No. |
|---|---|
| Fucosyltransferase III (FUT3; FT3) | BC108675 |
| Fucosyltransferase IV (FUT4; FT4) | BC136374 |
| Fucosyltransferase V (FUT5; FT5) | BC140905 |
| Fucosyltransferase VI (FUT6; FT6) | BC061700 |
| Fucosyltransferase VII (FUT7; FT7) | BC074746 |
| Fucosyltransferase IX (FUT9; FT9) | BC001879 |

As used herein, the notation for a fucosyltransferase should not be construed as limiting to the nucleotide sequence or the amino acid sequence. For example, the notation of Fucosyltransferase VII, FTVII, FT7, FUTVII or FUT7 are used interchangeably as meaning the nucleotide, amino acid sequence, or both, of Fucosyltransferase VII. According to some embodiments, cells are contacted by one or more of the α(1,3)-fucosyltransferase family members to enforce fucosylated lactosaminyl glycans.

In some embodiments, fragments of α(1,3)-fucosyltransferase family members are contacted with a cell. For example, a peptide/nucleotide having at least 50%, 60%, 70%, 80%, 90%, 95%, 96%, 97%, 98%, or 99% identity to an α(1,3)-fucosyltransferase family member is contacted with a cell. As used herein, the term “identity” and grammatical versions thereof means the extent to which two nucleotide or amino acid sequences have the same residues at the same positions in an alignment. Percent (%) identity is calculated by multiplying the number of matches in a sequence alignment by 100 and dividing by the length of the aligned region, including internal gaps.

In some embodiments, the cells may be contacted with the desired fucosyltransferase via exofucosyltation using, for example, the methods disclosed herein. U.S. Pat. Nos. 7,875,585 and 8,084,236, (which disclosures are expressly incorporated by reference as if recited in full herein) provide non-limiting examples of compositions and methods for ex vivo modification of cell surface glycans on a viable cell, which may be used to enforce expression of fucosylated lactosaminyl glycans on a cell according to the present disclosure. In some embodiments, the cells may be contacted with a purified glycosyltransferase polypeptide and a physiologically acceptable solution, for use together with appropriate donor nucleotide sugars in reaction buffers and reaction conditions specifically formulated to retain cell viability. In some embodiments, the physiologically acceptable solution may be free or substantially free of divalent metal co-factors, to such extent that cell viability is not compromised. In these and other embodiments, the cells may be contacted with a solution that is also free or substantially free of stabilizer compounds such as for example, glycerol, again, to such extent that cell viability is not compromised. Glycosyltransferases of the present disclosure include for example, one or more fucosyltransferase. In certain embodiments, the fucosyltransferase is an α(1,3)-fucosyltransferase such as an α(1,3)-fucosyltransferase III, α(1,3)-fucosyltransferase IV, an α(1,3)-fucosyltransferase V, an α(1,3)-fucosyltransferase VI, an α(1,3)-fucosyltransferase VII, or an α(1,3)-fucosyltransferase IX. However, it should be understood that fucosyltransferases other than these, for example the α(1,3)-fucosyltransferase from H. Pylori, could possess requisite capacity to install fucose in α(1,3)-linkage to GlcNAc and thus convert a Type 2-α(2,3)-sialylLacNAc into sLeX; as such, it should not be viewed that the exofucosylation reaction is limited to use of those α(1,3)-fucosyltransferase described herein.

In some embodiments, glycans are modified on the surface of a cell by contacting a population of cells with one or more glycosyltransferase compositions described above. In some embodiments, the cells are contacted with the glycosyltransferase composition together with an appropriate nucleotide sugar donor (e.g., GDP-fucose) under conditions in which the glycosyltransferase has enzymatic activity. For example, cells may be incubated for 60 min at 37° C. in fucosyltransferase reaction buffer composed of Hank's Balanced Salt Solution (HBSS) (without Ca²⁺ and Mg²⁺) (Lonza) containing 20 mM HEPES (Lonza), 0.1% human serum albumin (HSA) (Grifols, Barcelona, Spain), 30 μg/ml fucosyltransferase, and 1 mM GDP-fucose. Glycan modification according to this method results in cells according to the present disclosure that have at least 70%, 75%, 80%, 85%, 90%, 95%, 96%, 97%, 98%, 99% or more viability at 24 hours or more after treatment. In one embodiment, for example, the cells of the present disclosure have at least 70% viability at 48 hours after treatment. In one such embodiment, for example, the cells of the present disclosure have at least 75% viability at 48 hours after treatment. In one embodiment, for example, the cells of the present disclosure have at least 80% viability at 48 hours after treatment. In addition, the phenotype of the cells of the present disclosure (other than the glycan modification) is preferably preserved after treatment. By preserved phenotype, it is meant the cell of the present disclosure maintains its native function and/or activity. For example, if the cell of the present disclosure is an HSPC, its relevant multipotency or oligopotency or unipotency is maintained, as would be characteristic of that particular stem cell type.

According to some embodiments, glycosyltransferases are contacted with cells of the present disclosure in the absence of (or substantially in the absence of) divalent metal co-factors (e.g. divalent cations such as manganese, magnesium, calcium, zinc, cobalt or nickel) and stabilizers such as glycerol. In some embodiments, a purified glycosyltransferase polypeptide and a physiologically acceptable solution free or substantially free of divalent metal co-factors is used to enforce a desired glycosylation pattern. Such a composition is free or substantially free of stabilizer compounds such as for example, glycerol, or the composition contains stabilizers at levels that do not affect cell viability. The glycosyltransferases used with solutions that are free or substantially free of divalent metal cofactors include for example, α(1,3)-fucosyltransferases such as an α 1,3 fucosyltransferase III, α 1,3 fucosyltransferase IV, an α 1,3 fucosyltransferase VI, an α 1,3 fucosyltransferase VII, or an α 1,3 fucosyltransferase IX. According to some embodiments, the glycosyltransferase is biologically active. As used herein “biologically active” means that the glycosyltransferase is capable of transferring a sugar molecule from a donor to acceptor. For example, a glycosyltransferase according to the present disclosure is capable of transferring 0.1, 0.2, 0.3, 0.4, 0.5, 1.0, 1.5, 2.0, 2.5, 5, 10 or more pmoles of sugar per minute at pH 6.5 at 37° C. In some embodiments, the contacting of a glycosyltranferase with a cell occurs in a physiologically acceptable solution, which is any solution that does not cause cell damage, e.g. death. For example, the viability of the cell is at least 80%, 85%, 90%, 95%, 96%, 97%, 98%, 99% or more after treatment with the compositions of the invention. Suitable physiologically acceptable solutions include, for example, Hank's Balanced Salt Solution (HBSS), Dulbecco's Modified Eagle Medium (DMEM), a Good's buffer such as a HEPES buffer, a 2-Morpholinoethanesulfonic acid (MES) buffer, or phosphate buffered saline (PBS).

### Methods of Treatment/Administration of Cells

According to some aspects, the present disclosure provides methods of selecting HSCs on the basis of high sLeX expression (e.g., CD34+CD38−sLeX^{high} cells), or any other permutation in the grouping of markers that serve to define a human HSC in further combination with the marker “sLeX^{high}”. e.g., CD34+CD38−CD90+CD45RA−sLeX^{high}h cells or CD34+CD38−CD90+CD45RA−CD49f+sLeX^{high} cells) and then administering the sLeX^{high} HSCs to reconstitute bone marrow in a patient in need thereof.

Any of the cells described herein can be administered as freshly isolated cells, or following expansion in vitro, or cryopreserved and stored for administration subsequently. In some embodiments, the method comprises selecting a cell as disclosed herein, expanding the selected cell in suitable culture media and administering the expanded cells to the patient in any conventional manner. In some such embodiments, “reconstituting bone marrow” means restoration of all or a portion of the bone marrow in a patient suffering from a disease in which normal bone marrow function has been compromised. Non-limiting examples of such diseases include aplastic anemia, myelodysplastic syndromes (MDS), paroxysmal nocturnal hemoglobinuria (PNH), myelofibrosis, and blood cancers, such as leukemia, lymphoma, and myeloma. Thus, as used herein, “reconstituted” means that the transplanted cells (such as HSCs) are able to successfully engraft in the host and differentiate into all the cell lineages typically found in or derived from bone marrow.

According to some aspects, the discovery that certain subsets of HSPCs (e.g., MEPs) characteristically lack expression of sLeX specifically due to underfucosylation of terminal Type 2-α(2,3)-sialylLacNAc units, provides a method to enable engraftment of such cells by α(1,3)-exofucosylation of the respective HSPC surface to enforce sLeX expression. Thus, the present disclosure provides methods of correcting the deficiency of sLeX expression on cells such as MEPs, and/or on unipotent megakaryocytic or erythroid precursors derived therefrom, thereby greatly enhancing engraftment of such cells for treatment of conditions marked by marrow failure states, especially involving thrombocytopenia or anemia, respectively.

In some aspects, this disclosure provides a method for treating hematopoietic disorders, cancer, and, more generally, disorders amenable to treatment with stem cells (i.e., stem cell therapy) in a mammal, comprising administering to the mammal a composition comprising the cells isolated according to the methods described herein. According to some aspects, the present disclosure provides increasing the engraftment potential of HSCs and other HSPC subsets, by administering the cell population that is enriched for high cell surface expression of sLeX, thereby increasing the engraftment potential of that cell inoculum.

In some embodiments, the discovery of a wide range of sLeX expression within hematopoietic cells as a function of the type of HSPC provides for more specific identification of such subsets. Methods employing the quantification of cell surface sLeX expression thereby allows for more robust isolation of substantially homogenous compositions of subsets of human HSPCs, including cells comprising human HSCs, GMPs, and MEPs. The human HSCs and/or other subsets of HSPCs have clinical applicability for improved therapy of a variety of diseases/conditions, including, but not limited to, conditions requiring the replacement of certain cell types, the regeneration of hematopoietic elements, or the engraftment of genetically-modified cells. As a set of non-limiting examples, improved isolation/collection of HSCs would be critical for success of HSCT, for treatment of aplastic anemia, for HSC-based genetic correction/gene editing of hemoglobinopathies followed by HSCT, for HSC-based genetic correction/gene editing of immune deficiency conditions followed by HSCT, or for HSC-based genetic manipulation/gene editing of cell surface molecules that allow entry and infection of pathogens into hematopoietic cells followed by HSCT (e.g., eliminating expression of CCR5 on HSCs to prevent HIV entry/infection, followed by HSCT of CCR5-deficient HSCs that would thus generate CCR5-deficient leukocytes). In addition, for example, improved methods to isolate GMPs and MEPs on basis of sLeX expression levels would be useful for treatment of delayed engraftment following HSCT and/or for treatment of marrow failure states wherein production of platelets or red cells (erythrocytes) or myeloid cells (neutrophils or monocytes) is deficient. Once isolated, HSCs or other HSPC subsets could be expanded, to provide requisite numbers of cells for any clinical indication.

The capability to enrich HSCs by use of just three markers—CD34⁺CD38−sLeX^{high}—is highly advantageous in facilitating the collection of adequate numbers of HSCs for in vitro genetic manipulation/modification of the cells. Subsequent to such manipulations, the ability to enforce sLeX expression by cell surface α(1,3)-fucosylation would serve to enhance engraftment of the pertinent HSCs or other HSPC subsets, as needed. Genetic treatment of either autologous or allogeneic stem cells may be appropriate, depending on the clinical indication. For example, introduction of a wild-type gene into cells or gene-editing of the pertinent genetic mutation into autologously-derived HSCs, followed by administration of such cells in the form of HSCT, would be curative for a wide variety of hemoglobinopathies, as well as other genetic diseases such as osteopetrosis, leukocyte adhesion disorders (e.g., LAD I, LAD II, etc.), or more generalized immune deficits such as adenosine deaminase deficiency, recombinase deficiency, and others of the like. One could also employ this approach to introduce “resistance genes” to enable survival/persistence of HSPCs and other hematopoietic cells (e.g., erythrocytes lacking G6PDH) in patients undergoing treatment of cancer or infectious diseases. Diseases wherein the pathobiology involves lack of production of given protein (or class of proteins) could be corrected by introduction of the responsible regulatory sequence(s) so that a hematopoietic cell type could produce that protein under appropriate physiologic conditions. Alternatively, removal of a particular cell surface protein from a pertinent hematopoietic cell may be desired, such as removal of CCR5 to prevent HIV infection, or elimination of a particular TCR construct to prevent a relevant autoimmune disease or other immune disorder. These illustrative examples are not intended to be viewed as limiting to the wide scope of therapeutic advances that can be enabled by collection of high-quality HSCs using the 3-marker combination of CD34+CD38−sLeX^{high} in sufficient numbers to enable gene manipulations. Indeed, in some embodiments, for many indications, the use of a 2-marker panel consisting of just CD34+ and sLeX^{high} (i.e., CD34+sLeX^{high} cells) or just lin−sLeX^{high} HSPCs will suffice.

Administration of compositions, pharmaceutical compositions, including cell populations disclosed herein for therapeutic indications can be achieved in a variety of ways, in each case as clinically warranted, using a variety of anatomic access devices, a variety of administration devices, and a variety of anatomic approaches, with or without support of anatomic imaging modalities (e.g., radiologic, MRI, ultrasound, etc.) or mapping technologies (e.g., epiphysiologic mapping procedures, electromyographic procedures, electrodiagnostic procedures, etc.). The compositions, pharmaceutical compositions and cell populations of the present disclosure can be administered systemically, via either peripheral vascular access (e.g., intravenous placement, peripheral venous access devices, etc.) or central vascular access (e.g., central venous catheter/devices, arterial access devices/approaches, etc.). The compositions, pharmaceutical compositions and cell populations of the present disclosure can be delivered intravascularly into anatomic feeder vessels of an intended tissue site using catheter-based approaches or other vascular access devices (e.g., cardiac catheterization, etc.) that will deliver a vascular bolus of cells to the intended site. The compositions, pharmaceutical compositions and cell populations of the present disclosure can be administered directly into body cavities or anatomic compartments by either catheter-based approaches or direct injection into a pertinent anatomic site (e.g., intrabone/intramedullary (i.e., within the marrow itself). The compositions, pharmaceutical compositions and cell populations of the present disclosure can be introduced by direct local tissue injection, using either intravascular approaches (e.g., endomyocardial injection), or percutaneous approaches, or via surgical exposure/approaches to the tissue, or via laparoscopic/thoracoscopic/endoscopic/colonoscopic approaches, or directly into anatomically accessible tissue sites and/or guided by imaging techniques (e.g., intra-articular, intra-ocular, into spinal discs and other cartilage, into bones, into muscles, into skin, into connective tissues, and into relevant tissues/organs such as central nervous system, peripheral nervous system, heart, liver, kidneys, spleen, joints, eye, etc.). The compositions, pharmaceutical compositions and cell populations of the present disclosure can also be placed directly onto relevant tissue surfaces/sites (e.g., placement onto tissue directly, onto ulcers, onto burn surfaces, onto serosal or mucosal surfaces, onto epicardium, etc.). The compositions, pharmaceutical compositions and cell populations of the present disclosure can also administered into tissue or structural support devices (e.g., tissue scaffold devices and/or embedded within scaffolds placed into tissues, etc.), and/or administered in gels, and/or administered together with enhancing agents (e.g., admixed with supportive cells, cytokines, growth factors, resolvins, anti-inflammatory agents, etc.).

According to some embodiments, the compositions, pharmaceutical compositions and cell populations of the present disclosure are administered to the subject with an enforced expression of glycosylation. According to some embodiments, the enforced glycosylation on the surface of administered cells will aid in revascularization, in host defense (e.g., against infection or cancer) and/or in tissue repair/regeneration and/or mediate immunomodulatory processes that will dampen inflammation and/or prevent inflammation. According to some embodiments, the enforced glycosylation pattern guides delivery of intravascularly administered cells to sites of inflammation by mediating binding of blood-borne cells to vascular E-selectin expressed on endothelial cells at sites of inflammation. Moreover, whether cells are administered systemically, intravascularly, into the spinal canal and/or intraventricularly intrathecally, directly into body cavities or compartments, by direct local tissue injection, or by placement onto relevant tissue surfaces/sites, the enforced expression of sLeX on administered cells promotes lodgment of cells within the affected tissue milieu, in apposition to cells bearing E-selectin (i.e., endothelial cells) and/or L-selectin (i.e., leukocytes), respectively, within the target site. Thus, the spatial distribution and localization of administered cells within the target tissue is modulated by the enforced glycosylation on administered cells.

As used herein, the term a “therapeutically effective amount” of a cell is an amount sufficient to effect beneficial or desired results. In terms of treatment of a mammal, a “therapeutically effective amount” of a composition comprising the selected cells disclosed herein is an amount sufficient to regenerate respective blood cell types, or to treat, manage, palliate, ameliorate, or stabilize a condition, such as a bone marrow disease, in the mammal. A therapeutically effective amount can be administered in one or more doses. The therapeutically effective amount is generally determined by a physician on a case-by-case basis and is within the skill of one in the art. Several factors are typically taken into account when determining an appropriate dosage. These factors include age, sex and weight of the patient, the condition being treated, the severity of the condition and the form of the drug being administered.

The terminology used herein is for the purpose of describing particular embodiments only and is not intended to be limiting. As used in the specification and the appended claims, the singular forms “a,” “an,” and “the” include plural referents unless the context clearly dictates otherwise.

The following examples are provided to further illustrate the methods of the present invention. These examples are illustrative only and are not intended to limit the scope of the invention in any way.

### EXAMPLES

### Results

Phenotypic stratification of functional HSPC subsets

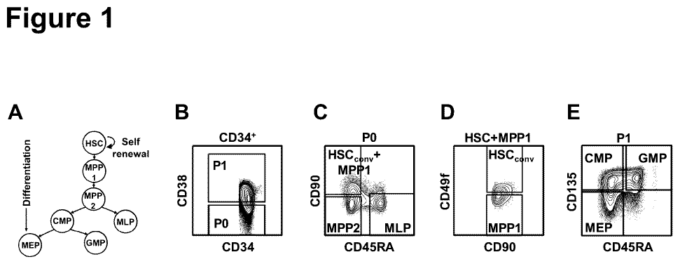

Seven functionally discrete HSPC subpopulations were identified based on a 6-marker panel that has been well-described (Doulatov et al., 2010; Notta et al., 2011; Sanada et al., 2016): CD34, CD38, CD90, CD45RA, CD49f, and CD135- or -CD123 (an interchangeable marker set) (FIG. 1; Supplemental Table 1). The most primitive, self-renewing human HSCs are extremely rare cells operationally characterized by their capacity to confer durable engraftment, defined via xenotransplantation as >20 week (>140-day) duration of human hematopoietic progeny in NOD/SCID/IL-2Rgamma^{null} (NSG) mouse hosts (“NSG repopulating” ability) (Notta et al., 2011); these human HSCs are phenotypically identified by a combined pentad of surface markers: CD34⁺CD38⁻CD90⁺CD45RA⁻CD49f⁺ (“HSC_{conv}”). Intermediate multi-potent progenitors (MPP) substage 1 (“MPP1”, defined as CD34⁺CD38−CD90⁺CD45RA⁻CD49f) and MPP2 (MPP substage 2, defined as CD34⁺CD38⁻CD90⁻ CD45RA⁻) are also able to produce all blood cell lineages, but these subsets contain ≤1% long-term NSG repopulating ability (Doulatov et al., 2010; Notta et al., 2011). Committed oligopotent progenitors include MLPs (multi-lymphoid progenitors), CMPs (common myeloid progenitors), GMPs (granulomonocytic progenitors), and MEPs (megakaryocyte-erythroid progenitors). MLPs, identified by marker phenotype CD34⁺CD38⁻CD90⁻CD45RA⁺, give rise to all lymphoid lineage cells (T cells, B cells, and NK cells) as well as some myeloid cells (monocytes, macrophages and dendritic cells) (Doulatov et al., 2010).

The CD34⁺CD38⁺ HSPC population (“intermediate and committed hematopoietic progenitor cells” (IC/HPCs)) comprises oligopotent and committed progenitors engendering only transient human myeloid engraftment in NSG xenotransplants (lasting <4 weeks post-transplantation, indicating lack of self-renewal ability) (Akashi et al., 2000; Doulatov et al., 2010; Manz et al., 2002). IC/HPCs include various subsets: (i) CMPs (CD34⁺CD38⁺CD135⁺CD45RA⁻, interchangeably, CD34⁺CD38⁺CD123⁺CD45RA⁻), oligopotent progenitors that differentiate into various non-lymphoid cells (monocytes, neutrophils, dendritic cells, megakaryocytes, and erythrocytes)(Majeti et al., 2007; Mazo et al., 1998); (ii) GMPs (CD34⁺CD38⁺CD135⁺CD45RA⁺, interchangeably, CD34⁺CD38⁺CD123⁺CD45RA⁺), cells generating only neutrophils, monocytes, macrophages, and dendritic cells; and, (iii) MEPs (CD34⁺ CD38⁺CD135⁻CD45RA⁻, interchangeably, CD34⁺CD38⁺CD123⁻CD45RA⁻), progenitors yielding megakaryocytes and erythrocytes.

The GMP Subset of Human HSPCs Displays the Highest Levels of Proteins that Mediate Marrow Engraftment

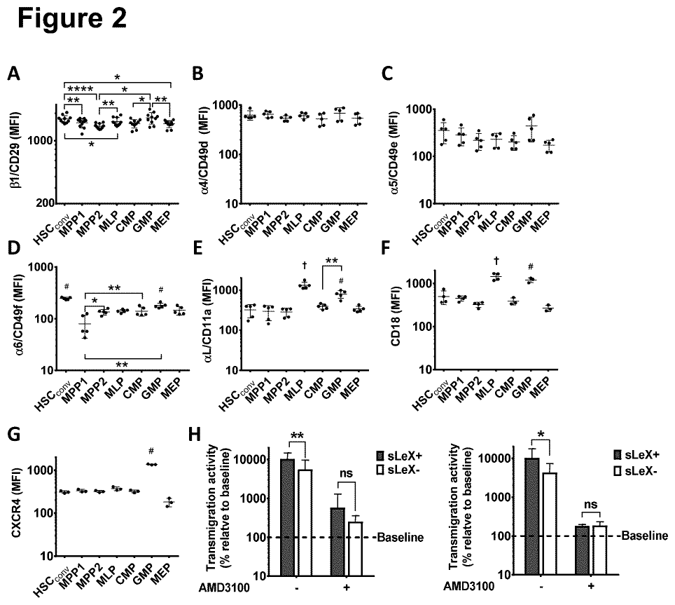

A number of HSPC membrane proteins function cooperatively with sLeX to promote osteotropism and consequent marrow engraftment: engagement of the chemokine receptor CXCR4 with chemokine CXCL12 triggers activation of integrin VLA-4 (and, to lesser extent, LFA-1) to enable HSPC firm adherence onto E-selectin-/VCAM-1-/ICAM-bearing marrow microvessels and successive extravasation, following which CXCR4/CXCL12-dependent chemotaxis and integrins VLA-4, VLA-5 and VLA-6 work collectively to enable HSPC lodgment within specialized hematopoietic growth niches. Using flow cytometry, expression of CXCR4 and of integrin subunits constituting VLA-4 (α4β1; CD49d/CD29), VLA-5 (α5β1; CD49e/CD29), VLA-6 (α6β1; CD49f/CD29), and LFA-1 (αLβ2) was assessed among the various CD34+ HSPC subsets (FIG. 2). Among the b1 integrins (FIGS. 2A-D), there are no significant differences in expression of VLA-4 and VLA-5 across HSPC subsets, whereas CD49f (a marker for HSC_{conv}s) shows highest levels in HSC_{conv} and GMP subsets (FIG. 2D). Both αL (CD11a) (FIG. 2E) and β2 (CD18) (FIG. 2F) subunits of LFA-1 are expressed at highest levels on MLPs and GMPs, while all other HSPC subsets display similar levels of LFA-1.

Notably, GMPs express ˜4.5-to-10-fold higher MFI for CXCR4 staining compared to all other HSPC subsets (FIG. 2G), and, altogether, these oligopotent HSPCs, not HSC_{conv}, display the highest levels of protein effectors of engraftment. To evaluate whether CXCR4 functionally differs among early human (multipotent) progenitor cells versus IC/HPCs, HSPCs were fractionated into subsets of CD34+CD38− (FIG. 2H, left panel) and CD34+CD38+(FIG. 2H, right panel) subsets, and also according to sLeX expression (sLeX+ versus sLeX−), for assessment of CXCL12-driven transmigration. CD34+CD38− and CD34+CD38+ subsets showed equivalent chemotactic responses, slightly more efficient among sLeX+ HSPCs compared to sLeX− HSPCs, and inhibitable by CXCR4-antagonist AMD3100 (FIG. 2H).

The HSPC Subset Defined by Markers CD34⁺/CD38−/CD90⁺/CD45RA−/CD49f+(“Conventional” HSCs (HSC_{conv})) Displays the Highest sLeX Levels and Most Potent E-Selectin Binding of any Human Cell

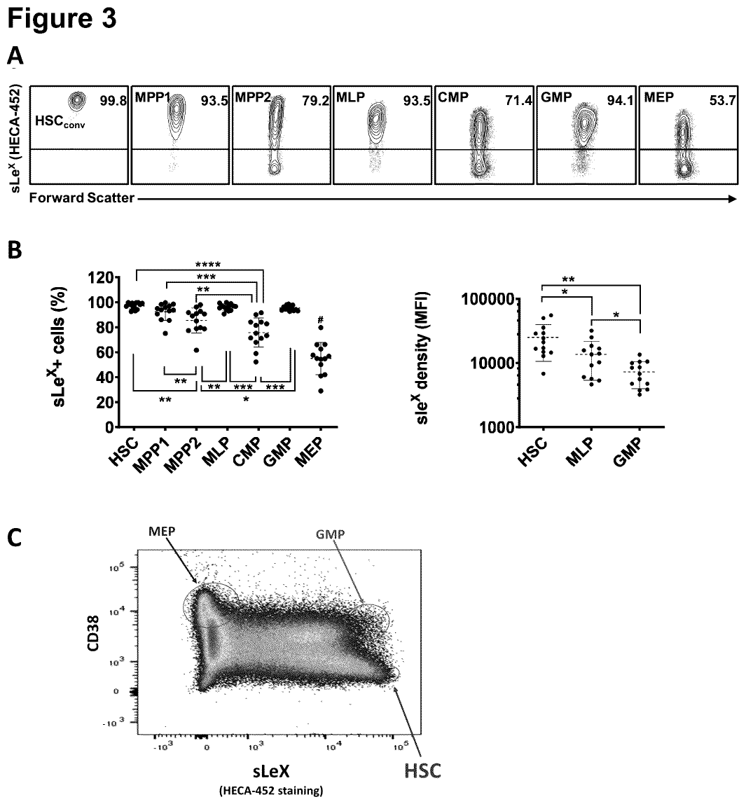

To assess sLeX levels within CD34+ HSPC subsets, multi-parameter flow cytometry was performed using mAb HECA452. As shown in FIGS. 3A and 3B, HSC_{conv}s uniformly express sLeX (˜100% positivity) at the very highest staining density (mean fluorescence intensity (“MFI”)) among all seven UCB HSPC subsets. MPP1 and MPP2 compartments contain fewer SLeX+ compared to HSC_{conv}s (MPP1 92%+6.8 and MPP2 85%±9.97) with a higher degree of variation in the percentage of sLeX+ cells than that of HSC_{conv}s (FIG. 3A and FIG. 3B left panel). MLPs are predominantly sLeX+(>93%) (FIG. 3B left panel), with lower surface density compared to HSC_{conv}s (FIG. 3B right panel).

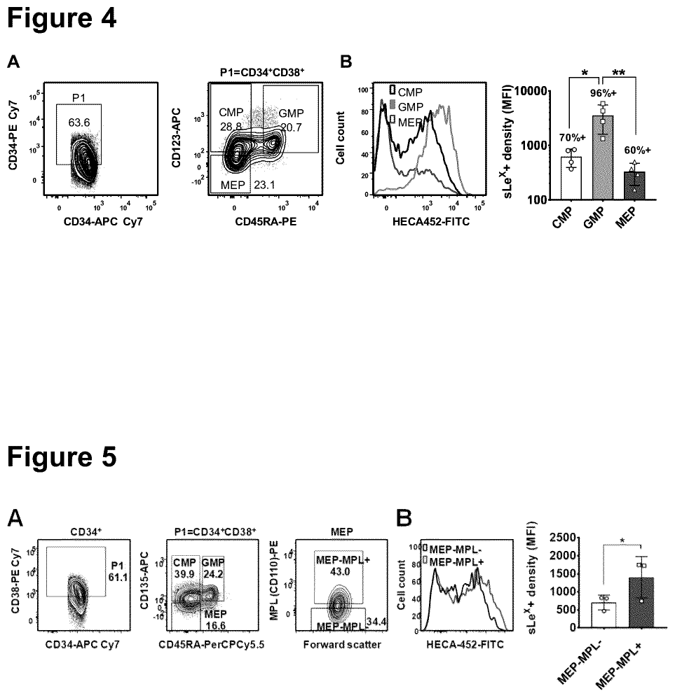

Within the CD34+CD38+ population (the IC/HPCs), whether using marker CD135 (as in FIGS. 3A and 3B) or CD123 (FIG. 4), identical differences in HECA452 staining were observed among CMP, GMP, and MEPs. A very high percentage of GMPs express sLeX (>93% sLeX+ cells) (FIG. 3B left panel and FIG. 4B), with surface density levels generally lower than that of MLPs and HSC_{conv}s (FIG. 3B right panel). CMPs and MEPs show wide variations in sLeX expression, with sLeX-positivity of ˜75%±1 and ˜55%+12, respectively (FIGS. 3A and 3B left panel). Notably, MEPs have the lowest fraction of sLeX+ cells (FIGS. 3A and 3B left panel) and also display low surface sLeX density (FIG. 3A and FIG. 4B). As shown in the flow cytometry dot blot of dual-color staining for CD38 versus sLeX in CD34+ HSPCs derived from either UCB or marrow (FIG. 3C), subsets of CD34+HSPCs can be distinguished and resolved on basis of respective CD38 and sLeX levels into HSCs (CD34+/CD38−/sLeX^{high}), GMPs (CD34+/CD38+/sLeX^{high}), and MEP CD34+/CD38+/sLeX^{-/low} (FIG. 3C).

To further characterize MEP sLeX expression, cells were subdivided by either presence or absence of the thrombopoietin receptor (“MPL”; CD110), respectively, into megakaryocyte progenitor-enriched (MEP-MPL⁺) or erythrocyte progenitor-enriched (MEP-MPL⁻) subsets (Sanada et al., 2016) (FIG. 5A); erythroid progenitors have the lowest sLeX expression levels of all HSPC subsets (FIG. 5B).

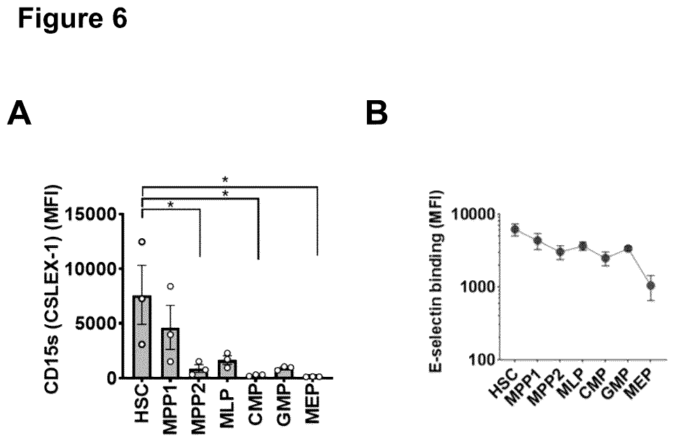

To further evaluate sLeX levels among human HSPC subsets, flow cytometry was performed with another anti-sLeX mAb, CSLEX-1 (FIG. 6A). Moreover, E-selectin binding was assessed by flow cytometry using E-selectin-Ig chimera (E-Ig) (FIG. 6B). HSC_{conv}, consistent with their utmost sLeX expression as measured using either HECA452 (FIG. 3) or CSLEX-1 mAb (FIG. 6A), have the greatest E-selectin binding of all human HSPCs (FIG. 6B).

Human HSPCs Express sLeX on Both Glycoprotein and Glycolipid Scaffolds

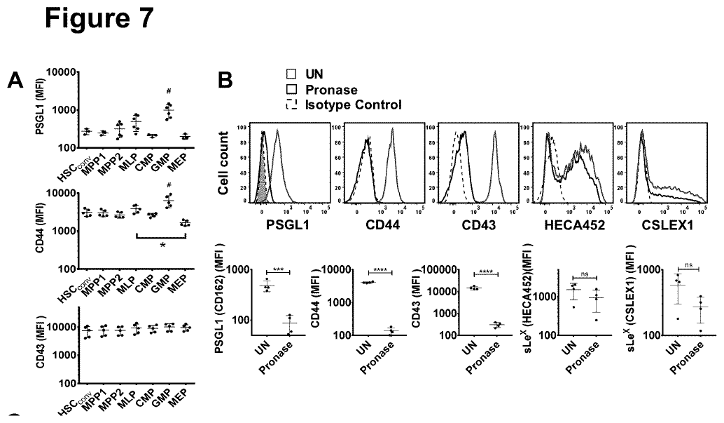

The sLeX motif is displayed either as N-linked or O-linked glycans on glycoprotein scaffolds, or as O-linked glycans on glycosphingolipid (GSL) backbones (Stolfa et al., 2016). The major glycoprotein carriers of sLeX are P-selectin glycoprotein ligand-1 (PSGL-1; CD162), CD44 (this sLeX-bearing CD44 glycoform is called “HCELL” (Dimitroff et al., 2001; Sackstein, 2016)), and CD43 (Merzaban et al., 2011). As shown in FIG. 7A, PSGL-1, CD44, and CD43 are expressed on all CD34⁺ human HSPC subsets, with variations in surface density among the HSPC subpopulations: (1) GMPs display PSGL-1 at highest density, followed by MLPs and MPP2s, while all other subsets display PSGL-1 at relatively low density; (2) GMPs also express the highest density of CD44 among HSPC subsets, while MEPs have the lowest CD44 levels; and (3) In contrast to both PSGL-1 and CD44, CD43 is highly expressed among all HSPC subsets. Thus, GMPs harbor the highest levels of glycoproteins that can display sLeX.

Membrane lipids (“glycosphingolipids” (GSLs)) of human myeloid leukocytes (Handa et al., 1997; Mondal et al., 2016; Nimrichter et al., 2008) and of mouse HSPCs (Winkler et al., 2012) have the ability to bind E-selectin. To assess whether GSLs on human CD34+ HSPCs display sLeX, cells were treated with a broad-range protease cocktail (pronase). Following pronase digestion, PSGL-1, CD44, and CD43 were undetectable by flow cytometry, yet there remained significant sLeX display as detected by HECA452 and CSLEX1 mAbs (˜50% residual binding) (FIG. 7B).

Stage-Specific Variation in α(1,3)-Fucosyltransferase Expression Dictates Differential sLeX Levels on Human HSPC Subsets

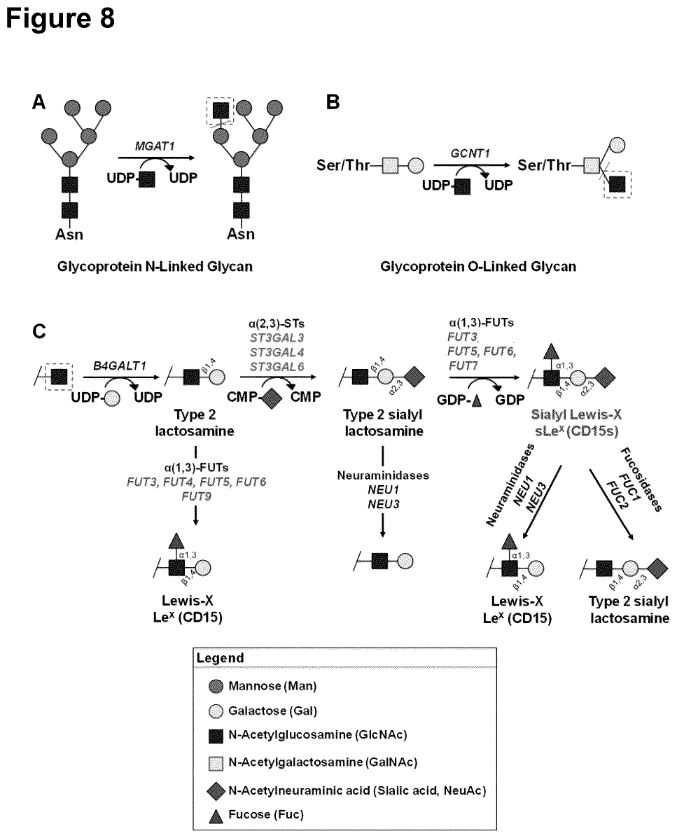

Cellular expression of glycan motifs is regulated by contrasting actions of two classes of enzymes: (1) Glycosyltransferases that catalyze glycan assembly via addition of monosaccharides in stereo-/regio-specific manner to pertinent acceptor structures; and (2) Glycosidases that program stereo-/regio-specific removal of relevant saccharide units from (assembled) glycan structures. Glycosyltransferase assembly of sLeX follows a strictly-ordered multi-step biosynthetic cascade (Mondal et al., 2015; Mondal et al., 2018; Nonomura et al., 2004; Stolfa et al., 2016; Yang et al., 2012); the pertinent human glycosyltransferases and glycosidases regulating sLeX display are shown in FIG. 8 (for details, see accompanying description of drawing/figure).

To unveil the mechanistic basis for the observed variability in sLeX levels among CD34+ HSPCs, levels of transcripts encoding the relevant glycosyltransferase and glycosidase enzymes were measured by qRT-PCR. Because quantities of HSC_{conv} and of MPP1 subpopulations were each insufficient for evaluating all pertinent transcripts, these subpopulations were pooled (i.e., HSC_{conv}+MPP1) (FIG. 1C), and this subset (comprising total CD34⁺CD38⁻CD90⁺CD45RA⁻ cells) was compared against MPP2, MLP, CMP, GMP, and MEP subsets (each subset separated as shown in FIGS. 1C and 1E). Gene expression levels for all glycan-modifying enzymes were evaluated as the % of transcript levels of housekeeping control genes (HCG), which comprised the geometric mean of GAPDH and R-actin transcript levels. Expression of pertinent genes was deemed “absent” if transcript levels were <0.1% of HCG, “low” if levels were >0.1% but <0.5% of HCG, “moderate” if >0.5% but <1% of HCG, and “high” if >1% of HCG.

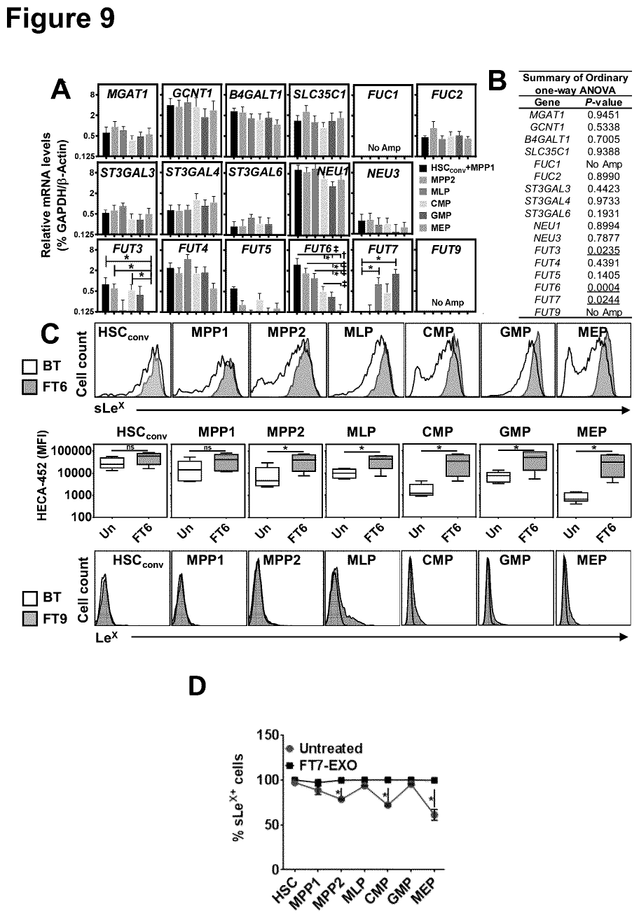

As shown in FIG. 9A, only minor variations were observed in expression of all genes that construct the backbone Type 2-LacNAc and core Type 2-α(2,3)-sialylLacNAc units across the six (i.e., HSC_{conv}+MPP1 vs MPP2 vs MLP vs CMP vs GMP vs MEP) subpopulations: all HSPC subsets express moderate levels of MGAT1 and high levels of GCNT1 and of B4GALT1 (FIG. 9A, top panel). Among α(2,3)-sialyltransferases (“α(2,3)-STs” (FIG. 9A, middle panel)) that install sialic acid in α(2,3)-linkage to terminal Type 2-LacNAc, ST3GAL3 is expressed at low levels in CMPs and GMPs, and at moderate levels in HSC_{conv}+MPP1, MPP2, MLP, and MEP subsets; ST3GAL4, the principal mediator of α(2,3)-sialylation of Type 2-LacNAc in human myeloid leukocytes (Mondal et al., 2015), is expressed at moderate levels within all subsets, with higher levels in CMPs and GMPs. Low levels of ST3GAL6 transcripts were observed in CD34+ HSPC subsets, with no transcripts in MEPs. Among the neuraminidases (sialidases), NEU1 is expressed at high levels and NEU3 is expressed at low levels across all HSPC subsets with negligible cross-subset differences (FIG. 9A, middle panel). Thus, variations in transcripts encoding enzymes that shape Type 2-α(2,3)-sialylLacNAc display do not account for fluctuations in sLeX expression among the CD34+ HSPC subsets.

In contrast, expression levels of genes encoding α(1,3)-fucosyltransferase (FT) isoenzymes vary dramatically within the human hematopoietic hierarchy (FIG. 9A, bottom panel). In humans, four distinct α(1,3)-FT isoenzymes (encoded by respective FUT genes), FT3, FT5, FT6, and FT7, can efficiently add Fuc in α(1,3)-linkage to GlcNAc within Type 2-α(2,3)-sialylLacNAc to create sLeX (FIG. 8). Of these enzymes, FT6 has the greatest potency in creation of sLeX, followed by FT7 (Mondal et al., 2018). Conspicuously, FUT7 transcripts are absent within the most primitive HSPC subsets, i.e., the HSC_{conv}+MPP1 and the MPP2 subsets. MEPs also lack FUT7 transcripts, while FUT7 levels are low in CMPs, moderate in MLPs, and high in GMPs. However, FUT6 transcript levels are strikingly high (>3.0% of HCG) in the HSC_{conv}+MPP1 population, with steady decrease along the differentiation hierarchy, i.e., HSC_{conv}+MPP1>MPP2>MLP>CMP>GMP/MEP. Thus, FT6 and FT7 expression levels are inversed in the hematopoietic hierarchy, and, importantly, the pattern of changes in levels of FUT6 and FUT7 transcripts are each highly significant (FIG. 9B). FUT5 transcripts are absent in MLPs and GMPs, with low levels in HSC_{conv}+MPP1, MLP, CMP and MEP subsets (FIG. 9A, bottom panel). FUT3 is moderately expressed in the HSC_{conv}+MPP1, MPP2, and CMP subsets, low in GMPs, and absent in MLPs and MEPs. Among α(1,3)-FTs that predominantly fucosylate Type 2-LacNAc to create LeX in human cells (Mondal et al., 2018), FUT4 is expressed at relatively high levels among all HSPC subsets with exception of MEPs, and FUT9 is not expressed by any human CD34+ HSPC. Among fucosidases, FUC1 is absent and FUC2 is expressed at similarly levels in all HSPCs. The GDP-fuc transporter (SLC35C1) is expressed at comparably high levels across all subsets (FIG. 9A, top panel), indicating that variable Golgi availability of this substrate does not contribute to the observed differential sLeX levels. Therefore, discrete stage-specific changes in levels of transcripts encoding the various α(1,3)-FT isoenzymes regulate human HSPC sLeX display, and, notably, transcripts encoding FT6, the α(1,3)-fucosyltransferase with greatest efficacy in converting Type 2-α(2,3)-sialylLacNAc to sLeX, are extraordinarily prominent within the most primitive human HSPCs.

Development of Glycosyltransferase Acceptor-Product (GAP) Analysis to Detect and Quantify Levels of Type 2-α(2,3)-sialylLacNAc and Type 2 LacNAc Motifs on the Surface of Human CD34+ HSPC Subsets

Though transcript levels encoding pertinent glycosyltransferases offer insights on the ability of a particular cell population to create a given glycan motif, such information cannot predict whether the pertinent glycan will be expressed because we currently lack technologies capable of interrogating glycan biosynthesis in situ: (1) We lack the capacity to quantify the content/levels of pertinent glycosyltransferases (and of glycosidases) natively within the Golgi; (2) We lack the ability to measure the kinetics of glycosyltransferase reactions (and glycosidase reactions) within discrete Golgi microenvironments; and (3) We lack the ability to identify the microenvironmental distribution/location of such enzymes within the Golgi, and of their pertinent glycan acceptors/targets and their pertinent nucleotide-monosaccharide donors. Glycan assembly is not a template-driven process, but it is nonetheless a highly ordered process driven by precise, step-wise glycosyltransferase-mediated installation of relevant monosaccharides onto specific precursors, the “acceptor glycans”. The extent to which a cell dedicates its biosynthetic capability to create a given acceptor glycan can be measured if there is a reporter for the presence of that acceptor: by inference, any mAb or lectin that recognizes any given glycan is not just measuring the level of expression of just the target motif per se, it is measuring the level of expression of the component glycan structure(s) that, in each biosynthetic step, engender that motif. As such, to understand the molecular regulation of sLeX biosynthesis within a cell population, it is necessary to measure the extent to which cells of that population elect to create the acceptor for α(1,3)-fucosylation: the core Type 2-α(2,3)-LacNAc terminal glycan.

Though flow cytometry using anti-glycan mAbs and/or lectins can provide qualitative and quantitative information on the display of discrete glycan motifs on scarce cells, there are no mAb nor lectins that are specific for Type 2-α(2,3)-sialylLacNAc nor Type 2-LacNAc terminal glycans. To achieve the needed precise identification and quantitation of these structures among the various HSPC subsets, a new glycoanalytic technique, “Glycosyltransferase Acceptor-Product” analysis (GAP analysis) was crafted by exploiting the exquisite stereo- and regiospecificity of glycosyltransferases for their target acceptors (Mondal et al., 2018; Sackstein, 2009). This technique is readily applicable and generalizable for detection and quantification of a variety of glycan structures, the only requirements being the availability of a relevant glycosyltransferase that modifies the pertinent target glycan (the acceptor), together with the availability of either a mAb or a lectin that can identify the pertinently modified glycan (the product); alternatively, a bioassay capable of quantifying a functional activity of that product could substitute for mAb or lectins. The glycosyltransferase reaction directly informs on the presence of the target acceptor; accordingly, the fidelity of detection lies in the potency of the glycosyltransferase, and the reporter reagents (e.g., mAb and/or lectins) need not be highly specific but must be sensitive. Flow cytometry is used to quantify mAb-based and/or lectin-based staining of the relevant cell population for the presence of the relevant product glycan before (i.e., at baseline) and then after the glycosyltransferase reaction: the observed difference in the staining intensity (MFI) and the change in the percentage of cells expressing the product are the “GAP” values. Accordingly, to quantify the content of “free” terminal LacNAc units consisting of either (unfucosylated) acceptor Type 2-α(2,3)-sialylLacNAc or “free” (unsialylated) Type 2-LacNAc units, correspondingly, the human α(1,3)-fucosyltransferases most potent in creating sLeX (FT6) and LeX (FT9) were used (in presence of donor GDP-fucose, with all reagents in excess and all reactions run to completion) (FIG. 8). Flow cytometry using anti-sLeX and anti-LeX mAbs was then performed, in each case quantifying the amounts of cell surface levels of these epitopes before and after α(1,3)-exofucosylation for each human HSPC subset (FIG. 9C)). The respective post-reaction boost in the percent of cells bearing sLeX or LeX determinants reflects the baseline distribution of cells bearing (unfucosylated) acceptor Type 2-α(2,3)-sialylLacNAc and Type 2-LacNAc units, correspondingly; the post-reaction boost in sLeX or LeX MFI levels thus reflects the cell surface density of acceptor-type structural units within the pertinent cell subset. But, importantly, the post-reaction sLeX level is a reflection of the total amount of terminal Type 2-α(2,3)-sialylLacNAc trisaccharide structures that have been created and then presented on the surface of that cell population.

Following α(1,3)-exofucosylation, the HSPC subsets that prominently harbored sLeX−cells (predominantly, the CMP and MEP subsets) were uniformly converted to sLeX+ populations. The highest change in HECA452-reactivity (i.e., largest increase in sLeX, thereby reflecting the highest innate baseline Type 2-α(2,3)-sialylLacNAc membrane level) occurred within the MEP subset (˜40-fold increase), followed by CMPs (˜18-fold), then GMPs (˜6-fold), MPP2s (˜5-to-10-fold), and MLPs (˜3-fold) (FIG. 9C, top middle panels). Importantly, HSC_{conv}, and MPP1 cell populations, which bear innately high sLeX levels have only modest increases in HECA452 staining after FT6-mediated exofucosylation (HSC_{conv}˜1.7-fold; MPP1-2.5-fold), indicating that the overwhelming majority of Type 2-α(2,3)-sialylLacNAc units created by these cells are, natively, α(1,3)-fucosylated; this finding is consistent with the observed high transcript levels of FUT6 (the most potent α(1,3)-FT for sLeX biosynthesis) among these primitive CD34+ HSPCs. Strikingly, cell surface sLeX density following FT6-mediated exofucosylation is uniformly high among all subsets, matching the levels of native HSC_{conv}(middle panel, FIG. 9C). The finding FT6-treatment equalizes the level of sLeX expression across all CD34+ HSPC subsets indicates that human CD34+ cells have equivalent cell surface levels of total Type 2-α(2,3)-sialylLacNAc units (i.e., there is uniform sum total of unfucosylated Type 2-α(2,3)-sialylLacNAc PLUS sLeX motifs among all HSPC subsets). This result further underscores the potency of FT6 in generating sLeX, and corroborates the PCR data indicating that differential α(1,3)-fucosylation regulates sLeX expression on HSPCs.

As shown in FIG. 9C lower panel, FT9-mediated exofucosylation converted Type 2-LacNAc units into LeX on minor fractions of MLPs (˜30%), CMPs (˜15%), GMPs (˜10%), and MEPs (˜20%), in each case with extremely low density (low MFI), but no LeX was created among HSC_{conv}, MPP1, and MPP2 subsets. Thus, the cell membranes of HSC_{conv}, and MPP1 compartments have a paucity of “free” Type 2-α(2,3)-sialylLacNAc acceptors (i.e., Type 2-α(2,3)-sialylLacNAc units are essentially “saturated” with α(1,3)-fucose modifications) and are completely devoid of “free” Type 2-LacNAc (unsialylated) units, whereas oligopotent progenitor populations have significant levels of “free” Type 2-α(2,3)-sialylLacNAc (i.e., acceptor units convertible to sLeX by FT6) and measurable, but relatively, minor amounts of “free” Type 2-LacNAcs. The findings that there are uniformly high levels of total Type 2-α(2,3)-sialylLacNAc glycans across all HSPC subsets explains the lack of native LeX expression on CD34⁺ HSPCs despite high transcript levels of FUT4 (FIG. 3C): i.e., essentially all LacNAc units are terminally modified with sialic acid in α(2,3)-linkage. In short, these results provide firm evidence that α(1,3)-fucosylation is the limiting factor regulating the variations in sLeX levels among CD34⁺ HSPCs, and, moreover, indicate that α(2,3)-sialylation of Type 2-LacNAc units is extremely efficient in all HSPCs, especially in HSC_{conv} and MPP1 subsets.

Enforced sLeX Display Markedly Accelerates Myeloid Engraftment by CD34+/CD38+ HSPCs

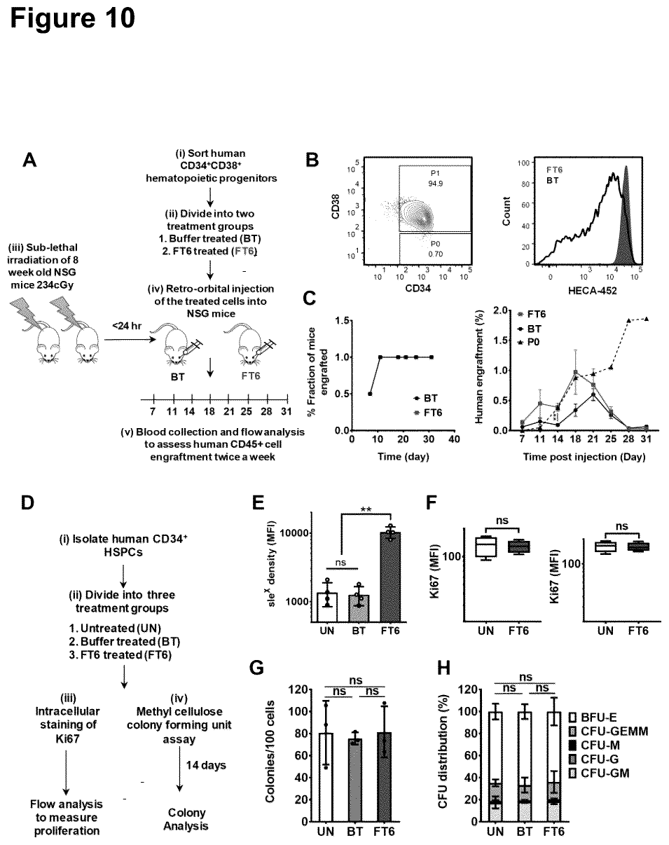

The immediate post-HSCT (“early-wave”) hematopoietic recovery is mediated by the IC/HPCs (i.e., CD34⁺CD38⁺ HSPCs) (Akashi et al., 2000; Manz et al., 2002; Mayani et al., 1993; Sutherland et al., 1989). To evaluate the impact of α(1,3)-exofucosylation of CD34⁺CD38⁺ HSPCs on hematopoietic recovery, xenotransplants were performed in sub-lethally irradiated (225 cGy) NSG mice using buffer-treated (“BT” group) or FT6-treated (“FT6” group) human cells, and early-wave human engraftment was assessed by measuring human CD45⁺ cells in mouse peripheral blood between 7 to 35 days post-transplant (FIG. 10). Exofucosylation uniformly and markedly augmented sLeX expression on human CD34+/CD38+ HSPCs (FIG. 10B). By day-7 post-transplant, low levels of human CD45⁺ cells were found in 50% of mice in both BT and FT6 groups; by day 11, all mice from both groups engrafted (FIG. 10C, left panel). Human CD45⁺ cells were found at higher abundance in peripheral blood from mice that received FT6-treated CD34⁺CD38⁺ at all time points (FIG. 10C, right panel). Importantly, compared to mice receiving BT cells, significantly higher human cell engraftment was observed within the FT6 group as early as day 11 post-transplant. The FT6 group maintained significantly higher human cell engraftment, peaking earlier (day 18) than that of the BT group (day 21). Human hematopoietic progeny formed by both FT6 and BT CD34⁺CD38⁺ progenitors disappeared by Day 28.

To examine whether the observed improved engraftment following exofucosylation reflects qualitative differences in HSPC biology, proliferation analyses and colony-forming unit (CFU) assays were performed on human CD34+ HSPCs that were untreated, BT-treated, or FT6-treated (FIG. 10D); FT6-treatment markedly elevated sLeX expression of CD34+ HSPCs (FIG. 10E). No significant difference in staining for Ki67 was observed between untreated and FT6-exofucosylated HSPCs (FIG. 10F), indicating that exofucosylation does not alter HSPC proliferation. CFU assays using semi-solid methylcellulose media containing cytokines supporting multi-colony formation (G-CSF, GM-CSF, and EPO) showed that untreated (UN), BT, and FT6-treated HSPCs create all colonies with equal efficiency (FIG. 10G), with no difference in the distribution of CFU types (FIG. 10H). Altogether, these findings indicate that the level of HSPC sLeX expression correlates with engraftment capability, but that sLeX expression does not in itself impact HSPC clonogenic activity.

### RNAseq Data Indicate that sLeX^{high}CD34+/CD38− HSPCs Transcriptionally Mirror HSPC_{conv}s

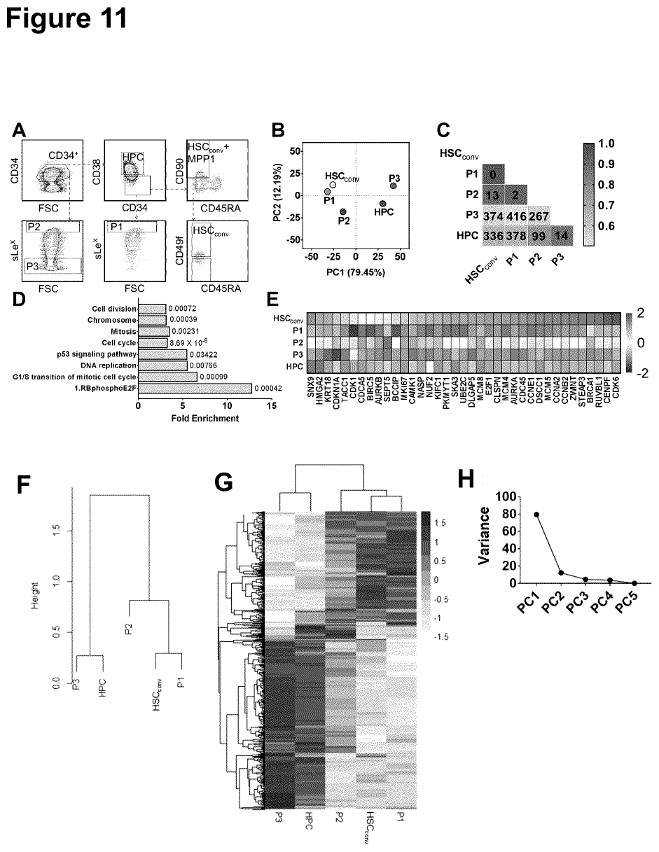

Gene expression profiles derived by RNAseq analysis provide critical information on cellular biological activities and are being increasingly used to identify distinct cell populations. To gain greater understanding on the role of sLeX as a biological marker of subpopulations of human HSPCs, RNAseq analysis of five isolated subsets of CD34⁺ HSPCs was undertaken: (1) “P1”−CD34⁺CD38⁻sLeX^{high} with “sLeX^{high}”, comprising the top 10% fraction of the sLeX-stained cells (i.e., the “high” MFI fraction); (2) “P2”−CD34⁺sLeX^{high} (3) “P3”−CD34⁺sLeX^{low/-} (comprising cells with the lowest 10% fraction of sLeX-stained cells); (4) HSC_{conv}s (CD34⁺CD38⁻CD90⁺CD45RA⁻CD49f⁺); and (5) “HPC”−CD34⁺CD38⁺ (see FIG. 11A). 459 differentially expressed genes (P<0.05) identified by DESeq2 differential expression analysis algorithm (Love et al., 2014) were interrogated. Unsupervised hierarchical clustering identified similar relationships between tested HSPC subpopulations (FIG. 11B-G). Principal component analysis (PCA) revealed that HSC_{conv}s, P1, and P2 subsets form transcriptionally similar clusters, while P3 clusters separately on the principal component space, in close vicinity to HPCs (FIG. 11B). To get a snapshot of the transcriptional differences between each HSPC subset the genes having significantly different expression by pair-wise comparison were evaluated (false discovery rate (FDR)<0.05, moderated t-test with Benjamini and Hochberg correction) (FIG. 11C). As shown in FIG. 11C and reviewed in Supplemental Table 2, HSC_{conv}s and CD34⁺CD38⁻sLeX^{high} cell (“P1”) exhibit extremely similar gene expression profiles (Pearson's correlation coefficient (PCC) 0.95) with no significant gene transcript differences, while only 13 gene transcripts were significantly different between P2 and HSC_{conv} (FIG. 10C). HPCs had distinctly different transcriptional program compared with HSC_{conv}(336 significantly different gene transcripts, PCC=0.66), P1 (378 differences, PCC=0.65), and P2 (99 differences, PCC=0.76) subsets. The P3 subset shows remarkable transcriptional resemblance with HPCs, differing only by 14 gene transcripts (PCC=0.87). DAVID (Database for Annotation, Visualization and Integrated Discovery) analysis (Huang et al., 2008; 2009), performed on the 378 genes differentially expressed between P1 and HPC subsets, reveals significant enrichment (FDR<0.05, Fisher's exact test with Benjamini-Hochberg multiple testing correction) of genes involved in cellular proliferation (cell division, mitosis, cell cycle, DNA replication, G1/S transition of mitotic cell cycle, P53 signaling pathway, and 1.RBPhosphoE2F categories) and in chromosomal organization (“Chromosome”) (FIG. 11D). While HPCs show robust expression of cell cycle drivers CDK1 and CDK6, and proliferation marker MKI67, P1 shows higher expression of the canonical cyclin dependent kinase (CDK) inhibitor CDKN1a (FIG. 11E). Within the P1 population, gene set enrichment analysis (GSEA) shows significant representation of genes reported to be expressed within the human HSC compartment (Eppert et al., 2011; Georgantas et al., 2004), while robust enrichment of the hematopoietic progenitor gene sets (Eppert et al., 2011) was observed in the P3 subset. These findings indicate that cells that express the highest levels of sLeX possess a parallel gene expression program to that of HSC_{conv}s, while the sLeX^{low/-} CD34⁺ HSPCs express similar transcriptional profile as HPCs (CD34⁺CD38⁺ HSPCs). Collectively, our data indicate that the expression level of sLeX alone is able to broadly distinguish between two transcriptionally distinct HSPC subsets within the CD34⁺ cell compartment.

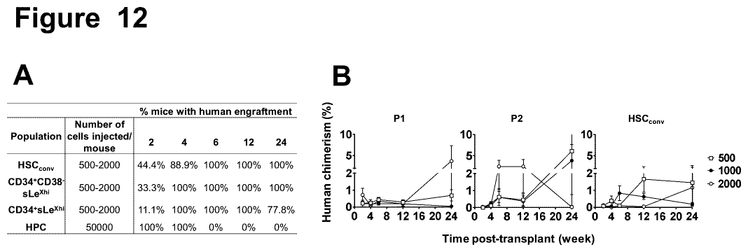

To examine whether the HSPC subpopulations isolated on basis of CD34 expression together with high sLeX expression (i.e., the P1 (CD34⁺CD38⁻sLeX^{high}) or P2 (CD34⁺sLeX^{high}) populations) are able to engender durable hematopoietic engraftment in recipient mice, 500-2000 cells comprising the respective HSC_{conv}, P1, P2, or HPC subpopulations were transplanted into sub-lethally irradiated NSG recipients, and resultant human CD45Y cell engraftment was assessed (FIG. 12). Engraftment was monitored for 24 weeks, insofar as 6 months of human engraftment in mouse hosts is sufficient to confirm human HSC contribution(s) (Notta et al., 2011). Mice receiving HPCs had robust transient (2-4 weeks) human CD45+ cell engraftment without durable human hematopoiesis, whereas those receiving HSC_{conv} displayed human engraftment in 44% of mice within 2 weeks post-transplant, in 89% at 4 weeks, and all mice had durable human engraftment from 8-24 weeks post-transplantation. Mice receiving P1 cells engendered human CD45⁺ cell engraftment within 2 weeks in 33% of the recipients, and, by week 4 post-transplant, 100% of the recipient mice displayed durable human engraftment (FIG. 12A). Persistence of human engraftment was observed in all P1 recipients at 24 weeks, with the frequency of human CD45⁺ cells increasing steadily to ˜5% of total CD45⁺ cells within mouse blood (FIG. 12B). Transplantation of the P2 population also yielded significant human cell engraftment: 11% of recipients had circulating human CD45⁺ cells at week 2 post-transplantation, and 100% of recipients showed human cell engraftment from 4-12 weeks. Similar to recipients of P1 cells, the peak frequency of P2 engraftment was observed to be ˜5% of total blood CD45⁺ cells (FIG. 12B); however, in contrast to mice receiving P1 or HSC_{conv} cells, not all of the P2 recipients displayed human chimerism at 24 weeks (FIG. 12A). Collectively, these results indicate that the sLeX^{high} fraction of either total CD34+ or CD34+CD38− HSPCs is enriched for human HSCs.

High sLeX Expression Orchestrates Human HSPC Osteotropism

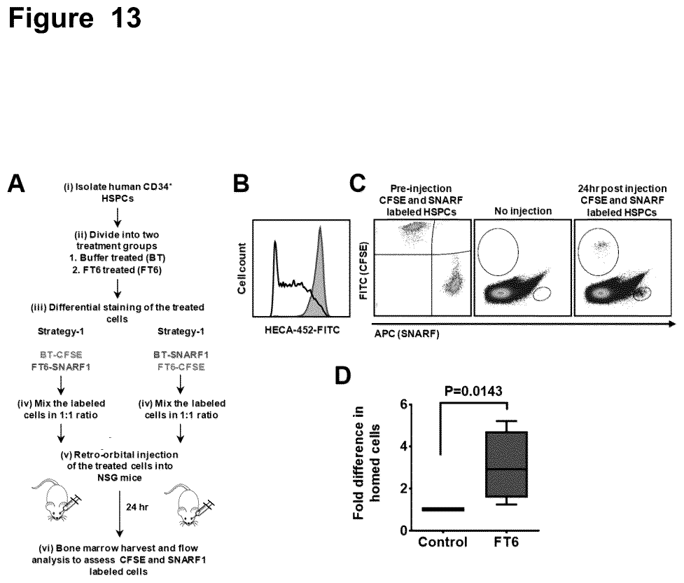

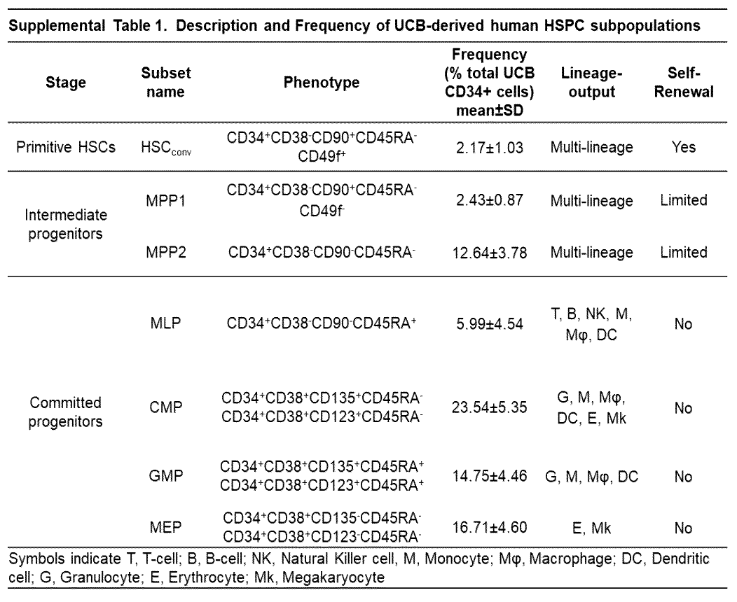

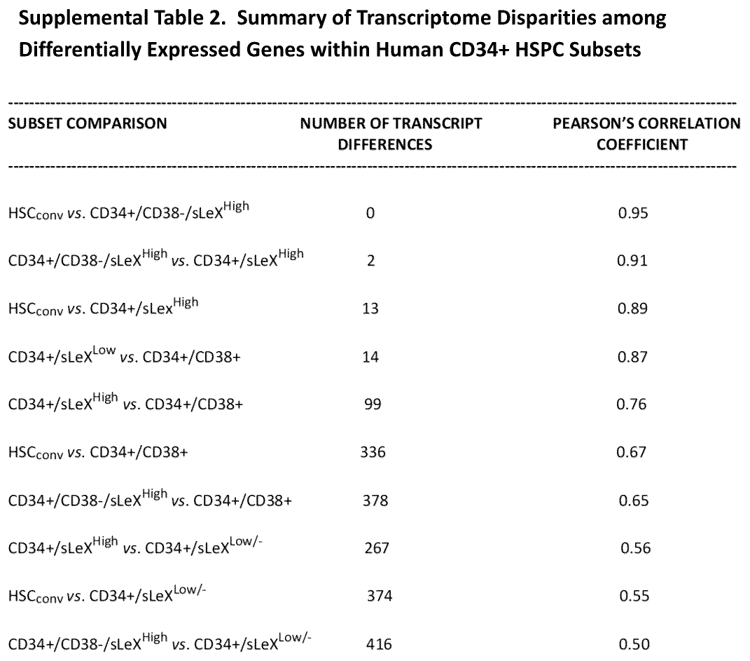

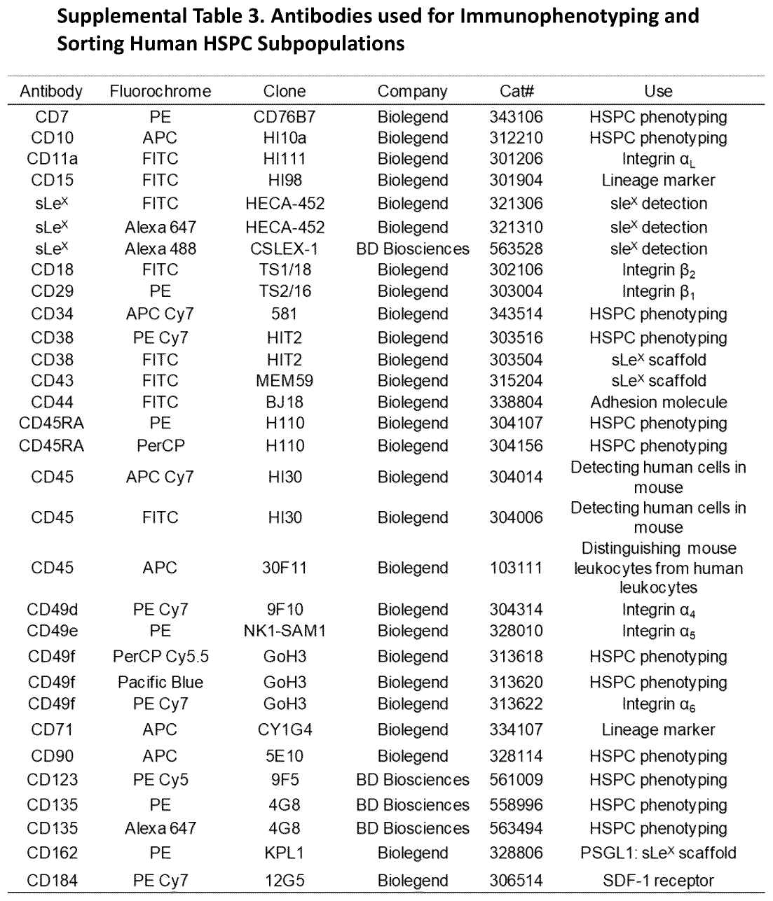

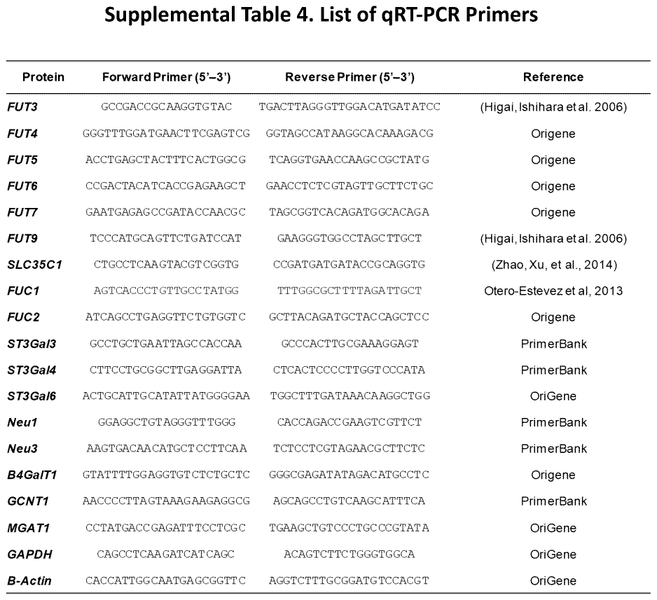

sLeX is known to serve as a mediator of osteotropism, but a variety of cell surface proteins (e.g., CXCR4 and integrins (e.g., VLA-4 and LFA1) are also potent mediators of osteotropism (and of engraftment, as well). Given the finding that human HSPC subsets display marked variations in expression of the sLeX glycan, yet display only modest variations in the protein effectors of osteotropism, the extent to which boosting sLeX expression alone would impact recruitment of human CD34+ HSPCs to marrow was evaluated. To this end, buffer-treated (BT, control) or FT6-exofucosylated (FT6) CD34⁺ HSPCs were differentially labeled with dyes CFSE or SNARF-1, and co-injected (1:1 mixture) into the retro-orbital sinus of NSG mice (FIG. 13A). FT6-generated α(1,3)-exofucosylation converted all cells to sLeX+ and produced a ˜7-fold increase in sLeX staining (FIG. 13B). At 24 hours post-transplantation, there was >3-fold higher marrow accumulation of FT6-exofucosylated HSPCs compared to that of BT (FIGS. 13C and 13D).

### Discussion

The results of our studies provide firm evidence that human HSCs, as defined via the pentad marker combination CD34⁺CD38⁻CD90⁺CD45RA⁻CD49f⁺ (Notta et al., 2011), are cells specialized to express high levels of the glycan motif sLeX. Our findings are in sharp contrast to results of others that have suggested that early-stage/multipotent human HSPCs have relatively lower sLeX expression compared to oligopotent progenitors (Hidalgo and Frenette, 2005; Katayama et al., 2003; Robinson et al., 2012; Robinson et al., 2014; Xia et al., 2004). However, no prior study examined a human HSPC subpopulation expressly enriched for HSCs. Our data show that sLeX expression varies markedly in a stage-specific fashion within the human hematopoietic hierarchy, yet, uniformly, human HSCs express the highest levels of sLeX of all HSPCs.

To elucidate the mechanistic basis of the extraordinary level of sLeX display on HSCs and the marked variability in sLeX expression among human HSPC subsets, qRT-PCR studies were undertaken to measure transcripts encoding glycosyltransferases and glycosidases that mediate creation and degradation, respectively, of this glycan motif. These studies show that levels for glycosyltransferases mediating assembly of the Type 2-α(2,3)-sialylLacNAc precursor of sLeX, and of its degradative enzymes, remain relatively stable within the hematopoietic hierarchy (FIG. 9A). However, striking differences were observed in the expression of α(1,3)-FUT genes across human HSPC subsets, suggesting that variable α(1,3)-fucosylation dictates the observed pattern of sLeX expression in HSPCs.

To obtain definitive evidence as to whether α(1,3)-fucosylation alone dictates variations in sLeX display among HSPCs, it was necessary to create a method to precisely quantify the level of expression of Type 2-α(2,3)-sialylLacNAc, and of its precursor disaccharide (Type 2-LacNAc), on the pertinent HSPC subsets. This new technique, “GAP analysis”, capitalizes on the inherently strict regio- and stereoselectivity of glycosyltransferases for their respective acceptor glycans, in combination with the quantitative power of flow cytometry to identify the pertinent reaction product. GAP analysis fills a number of existing technological and methodological gaps in our current ability to probe glycan structures: it is quantitative, extremely specific and sensitive, easy to employ, has high through-put, requires no sample preparation, is applicable for rare cell populations, and does not demand technologically complex equipment requiring specialized operators. In particular, it enables the precise detection and quantification of a pertinent cell surface glycan structure for which there exists no specific mAb nor lectin that can reproducibly and uniquely detect that structure. Though created in this study for the express purpose of quantifying levels of Type 2-α(2,3)-sialylLacNAc on rare cell populations, this method is readily generalizable for interrogation of a large number of other glycan motifs insofar as there are currently dozens of available glycosyltransferases that engender glycan products for which there are mAb reagents and/or lectins that can identify those products. Here, the expression of the pertinent precursor Type 2-α(2,3)-sialylLacNAc and Type 2-LacNAc glycans on the cell surfaces were measured by use of fucosyltransferases FT6 and FT9, respectively, to create the relevant epitopes sLeX and LeX. These studies show that the human HSC is essentially “saturated” with sLeX motifs with scarce “free” Type 2-α(2,3)-sialylLacNAc units that could be converted to sLeX by enforced α(1,3)-fucosylation (FIG. 9C). In sharp contrast, more mature HSPCs (IC/HPCs) natively display comparatively lower levels of sLeX and much higher levels of (unfucosylated) Type 2-α(2,3)-sialylLacNAc acceptors (FIG. 9C). Thus, exofucosylation can markedly enhance sLeX display on IC/HPCs, but not on HSCs, yielding more efficient IC/HPC osteotropism, with resultant improved (short-term) production of immediately-maturing progeny. Importantly, the fact that α(1,3)-exofucosylation confers upon any CD34+ cell an sLeX density parallel to that of native HSCs indicates that the sum total of (unfucosylated) Type 2-α(2,3)-sialylLacNAc plus sLeX motifs on the surface of all human CD34+ cells is similar, i.e., the content of total α(2,3)-sialylated lactosamines is relatively constant among all CD34+ subsets of the hematopoietic hierarchy. These data firmly establish that differential α(1,3)-exofucosylation is the key step regulating human HSPC sLeX biosynthesis. Importantly, in light of prior reports suggesting that early-stage HSPCs were deficient in sLeX expression, it had been posited that HSC osteotropism could be mediated by an inordinately high expression of one or several of cooperating proteins (e.g., CXCR4, VLA-4, LFA-1) that could compensate for the sLeX deficit (Katayama et al., 2003). The data here show that there is relatively little variation in expression of these proteins among HSPC subsets, and that GMPs, not HSCs, express the highest levels (FIG. 2). Still, exofucosylation-enforced sLeX expression significantly boosts IC/HPC osteotropism with resulting improved marrow recruitment (FIG. 13) and engraftment (FIG. 10).

Notably, a clinical trial of dual-unit UCB transplantation was undertaken wherein one of two transplanted human UCB units was exofucosylated (Popat et al., 2015). The exofucosylated UCB unit yielded markedly faster hematopoietic recovery, however, quite unexpectedly, at 100 days post-transplant (indicative of “long-term” engraftment mediated by HSCs), the exofucosylated and the unmanipulated UCB unit contributed equally to donor hematopoiesis. The paradoxical finding that exofucosylation of UCB human HSPCs enhances “short-term” but not “long-term” engraftment led to speculation that human HSC osteotropism proceeds independently of sLeX expression. However, now, our findings here solve the paradox as to why α(1,3)-exofucosylation improves short-term, but not long-term, engraftment in patients that receive exofucosylated UCB cells: HSCs have surfeit sLeX determinants and thus are natively specialized for osteotropism (and engraftment), whereas exofucosylation can significantly enhance sLeX expression on committed/oligopotent progenitors and thereby substantially improves both their osteotropism (and engraftment). Hence, exofucosylated UCB units contribute to faster short-term (i.e., IC/HPC-dependent) engraftment, but exofucosylation does not impact the kinetics of long-term (i.e., HSC-dependent) engraftment.

Owing to major species-specific differences in glycosyltransferases, the breadth and scope of sLeX display differs markedly between hematopoietic cells of humans and mice. Four genes in humans, FUT3, FUT5, FUT6, and FUT7, encode respective α(1,3)-fucosyltransferase isoenzymes that efficiently create sLeX by adding Fuc in α(1,3)-linkage to GlcNAc within terminal Type 2-α(2,3-sialylLacNAc units (Mondal et al., 2018); FIG. 8). The mouse genome lacks FUT3, FUT5, and FUT6, raising caution on extrapolating murine-based studies regarding the cell biology of sLeX expression to that of human cell biology; indeed, a comprehensive study of sLeX expression in murine HSPCs has shown that the most primitive mouse HSPCs, the “long-term HSCs” (LT-HSCs), are relatively deficient in sLeX expression (Al-Amoodi et al., 2022). Importantly, our findings here reveal that the most primitive human HSPCs (HSC_{conv}+MPP1 subsets) robustly express transcripts encoding FT6. FT6 is the most potent of all α(1,3)-fucosyltransferases for sLeX creation (Mondal et al., 2018) followed by FT7, a fucosyltransferase best known for mediating sLeX assembly on mature human leukocytes (Buffone et al., 2013; Homeister et al., 2001; Weninger et al., 2000). Our data here show that FUT7 expression is conspicuously repressed at the earliest stages of the human hematopoietic hierarchy (i.e., within HSC_{conv}s and MPPs); it is then sharply upregulated precisely at lympho-myeloid differentiation, with increasing expression at transition from CMP to GMP stages followed by a precipitous drop in MEPs (see FIG. 9A). In line with the paucity of sLeX display on MEPs, the levels of transcripts encoding α(1,3)-fucosyltransferases that can create sLeX are very low in this subset. Altogether, these findings are consistent with prior observations that the cytokine granulocyte-colony stimulating factor (G-CSF) selectively induces E-selectin ligand expression on late-stage human myeloid precursors and human mature granulocytes by upregulating FUT7 expression (Dagia et al., 2006). Therefore, lineage-biasing hematopoietic cytokines within distinct marrow microenvironmental growth niches can shape expression of the various α(1,3)-FUT genes.

Gene expression programs cellular function, and, as such, RNAseq analysis is being increasingly used to biologically define distinct cells and cell populations. To further assess how surface sLeX expression levels relate(s) to human HSC biology, comparative gene expression studies were performed of HSPC subsets expressing sLeX at highest levels (“sLeX^{high}” cells) and at lowest levels (“sLeX^{-/low}” cells) against that of HSCs (i.e., purified HSC_{conv}s). Gene set enrichment analysis (GSEA) performed on the 4731 curated gene sets of the MSigDB dataset reveals significant enrichment of the HSC_{conv}-specific genes within the sLeX^{high} HSPCs, in particular, the CD34⁺CD38-sLeX^{high} cells, and enrichment of the IC/HPC-specific genes in the CD34⁺sLeX^{-/low} cells: there is transcriptomic overlap between the CD38− subset of CD34+sLeX^{high} cells and HSC_{conv}, whereas the transcriptome of CD34⁺sLeX^{-/low} cells resembles that of oligopotent HSPCs. Importantly, results of xenotransplants corroborate these findings in that selection of the sLeX^{high} subset of CD34⁺ cells, and, even more prominently, the sLeX^{high} subset of CD34+CD38− cells, results in robust long-term human engraftment. Thus, selection of human HSPCs bearing the highest sLeX levels in itself enriches for self-renewing and multipotent HSPCs. Accordingly, multiple orthogonal experimental approaches—(1) flow cytometry studies indicating that HSC_{conv}s express the highest and most homogenous levels of sLeX of all CD34⁺ HSPCs (FIG. 3 and FIG. 6), (2) RNAseq analysis revealing that the transcriptome of CD34⁺CD38⁻sLeX^{high} HSPCs overlaps that of HSC_{conv}s (FIG. 10 and Supplemental Table 2), and (3) xenotransplantation studies showing that human CD34⁺CD38⁻sLeX^{high} HSPCs uniformly achieve durable human engraftment (FIG. 12)—provide strong evidence that the triad of markers CD34⁺CD38−sLeX^{high} suffices to both phenotypically and operationally identify human HSCs, and that the CD34+/sLeX^{high} fraction of human HSPCs can, in itself, provide a very highly HSC-enriched subpopulation of HSPCs.

The variable expression of sLeX among defined subsets of human CD34+ HSPCs highlights the utility of cell surface glycans in phenotyping developmental events (Lanctot et al., 2007). Specifically, since glycan expression is orchestrated by synergistic activities of several genes, the diversity of these motifs can provide “glycosignatures” reflective of stage- and lineage-specific cellular changes during embryonic development and cell differentiation processes (Enver et al., 2009). Notably, CD34 glycans themselves change distinctly during human hematopoietic cell maturation (Nielsen and McNagny, 2008). In mammals, glycan determinants are routinely used for identification and isolation of embryonic stem cells: indeed, the glycan motif LeX is the principal marker of murine embryonic stem cells (called “Stage-Specific Embryonic Antigen (SSEA)-1”), and, though human embryonic stem cells do not express this trisaccharide, Le^{X} is “CD15,” the primary marker defining human myeloid cells (Gadhoum and Sackstein, 2008; Gooi et al., 1983; Tao et al., 2004). In addition, the principal cell surface markers of human pluripotent stem cells (i.e., SSEA-3, SSEA-4, TRA1-60, and TRA1-81) are glycan epitopes (Lanctot et al., 2007; Muramatsu and Muramatsu, 2004; Schopperle and DeWolf, 2007). Importantly, recent data indicate that human mesenchymal stem cells, multipotent adult stem cells that also reside within bone marrow, possess a distinct glycosignature characterized by total absence of α(1,3)-fucosylated structures on the cell surface, with abundant expression of (unfucosylated) Type 2-α(2,3)-sialylLacNAc units (Mondal et al., 2018; Pachón-Peña et al., 2017). In this light, it is highly remarkable that all CD34+ HSPCs display similar levels of total Type 2-α(2,3)-sialylLacNAc structures on their surfaces, indicating that the Golgi biosynthesis of Type 2-α(2,3)-sialylLacNAc units is relatively constant during early stages of human hematopoiesis. There is mounting evidence that sLeX/E-selectin-receptor/ligand interactions within E-selectin-bearing microvascular hematopoietic niches play a critical role in HSPC maintenance/dormancy, proliferation, and differentiation (Winkler et al., 2012). Thus, variable Golgi addition of a single monosaccharide stereospecifically, i.e., α(1,3)-fucosylation, can tune hematopoietic development.

The isolation of human HSCs using the conventional pentad of markers, though certainly feasible, is both labor-intensive and technologically challenging, and target cell yields drop precipitously as a function of the number of markers needed to isolate any given cell population. As such, there remains a pressing need to further define markers that can readily identify human HSCs phenotypically. The results herein show that paramount sLeX expression serves as a glycosignature of human HSCs, provide a mechanistic understanding of the biosynthesis of this HSPC glycan motif within various stages of the human hematopoietic hierarchy, and unveil a new paradigm for isolation of human HSCs that depends on only three markers (CD34⁺CD38⁻sLeX^{high}). The high E-selectin binding capacity of the human HSC indicates that osteotropism is another innate feature of this stem cell, beyond the prototypical stem cell characteristics of self-renewal and multipotency. Importantly, in the context of emerging in vitro manipulations of human HSCs for therapeutic purposes (e.g., culture-expansion, genetic modification(s), etc.), this fundamental operational property must be preserved to achieve intended clinical outcomes. Further studies on the glycobiology of human hematopoiesis are warranted as this information is critical to not only understanding how in vitro manipulations could impact human HSPC properties/function, but, more broadly, to unraveling how changes in HSPC surface glycans mediate steady-state blood formation and how they converge with mutational, epigenomic, transcriptomic, and metabolomic variations to drive deranged human hematopoiesis (Trumpp and Haas, 2022).

### Methods

### Isolation of Human Hematopoietic Stem and Progenitor Cells

Human cells were obtained and used in accordance with the procedures approved by the Human Experimentation and Ethics Committees of Partners HealthCare. Discarded bags of umbilical cord blood (UCB) were obtained from the cell processing labs of the Dana-Farber Cancer institute and the MD Anderson Cancer Center (Houston, Texas). Total mononuclear cells (MNC) were isolated from UCB by Ficoll-Paque density gradient centrifugation. CD34⁺ HSPCs were purified using immuno-magnetic cell separation technology (STEMCELL Technologies, Vancouver Canada) according to manufacturer's protocol.

### Flow Cytometry and Cell Sorting

All monoclonal antibodies used for this study are listed in Supplemental Table 3. To stain cells for flow cytometry analysis or flow-assisted cell sorting, HSPCs were first incubated with Fc receptor blocking solution for 5 min at room temperature followed by incubation with staining cocktail containing relevant primary conjugated antibody mixture prepared in PBS containing 2% FBS. Flow cytometry analysis was done using either on BD FACS Canto or LSRII (BD, San Jose, CA). For multicolor experiments, compensation controls were established using UltraComp eBeads (Invitrogen, Carlsbad, CA), and spectral overlap was calculated using the automatic module of BD FACSDIVA. Relevant Fluorescence minus one (FMO) controls along with isotype controls were set up for gate setting (Supplemental figure S9). Flow cytometry data thus obtained were analyzed using FlowJo software (Tree Star, Ashland, Or). Cell sorting/subset isolation was performed using BD FACS Aria sorter. Viable cells were counted post-sort before further experimentation.

### Isolation of Total Cellular RNA

Total cellular RNA was purified from sorted subsets of UCB HSPCs using RNeasy micro kit (QIAGEN, Hilden, Germany) as per the manufacturer's instructions. The purified RNA was either converted to cDNA for quantitative RT-PCR experiments or utilized for RNA sequencing analysis.

### Transmigration Assay

CD34⁺ human HSPCs were suspended at 1×10⁶/ml in RPM11640 media supplemented with 25 mM HEPES, 10% FBS, and 1% penicillin/streptomycin (full RPMI). For negative control experiments 50 μM CXCR4 antagonist AMD3100 was added to the cell suspension. 100 μl of the cell-suspension (i.e., 1.5×10⁵ total cells) were seeded into uncoated 6.5 mm transwell chambers with 5.0 μm pore size (Costar). The transwells were placed into individual wells of a 24-well plate pre-loaded with 600 μl full RPMI (No SDF-1 control), full RPMI+125 ng/ml SDF-1 (SDF-1), or full RPMI+125 ng/ml SDF-1+50 μM CXCR4 antagonist (SDF1+AMD3100). Equal numbers of cells were added directly into the wells of the 24-well plate, without transwells (as counting controls). The plate was then incubated at 37° C. for 4 hours, after which, transwells were removed and the bottom surface of the transwell was gently rinsed with media from the bottom well. The number of cells in each well was quantified using flow cytometry by adding a known quantity of absolute counting beads (CountBright, Molecular Probes). Cell transmigration was determined using the following formula. Transmigration activity (ratio)=Calculated number of cells in the bottom well/number of cells in the corresponding counting control well. Baseline transmigration was calculated as indicated above, as the transmigration activity in the “No SDF-1” control well. The relative transmigration activities in the “SDF-1” and “SDF-1+AMD3100” groups were determined as follows. Relative transmigration activity (%)=100×(Transmigration in individual treatment group (SDF−1 or SDF1+AMD3100)/baseline transmigration).

### Quantitative RT-PCR

Total cellular RNA was reverse-transcribed using SuperScript VILO cDNA conversion kit (Invitrogen, Carlsbad, CA). Quantitative real time PCR (qRT-PCR) was performed with specific primers to amplify target genes (listed in Supplemental Table 4) using SYBR Select master mix (Applied Biosystems, Foster City, CA) and StepOne Plus PCR detection system (Applied Biosystems). PCR reactions for individual genes were performed in triplicate. Post-amplification, melt curve analysis was performed to ensure primer binding specificity. Geometric mean of GAPDH and R-Actin levels was used to normalize the expression of target genes (Vandesompele et al., 2002), and was calculated according to the following formula: Relative mRNA level (%)=2^{-ΔCt}X100, where Ct is the threshold cycle of amplification for each gene, and ΔCt=Ct_{traget gene}−Geometric mean of Ct_{GAPDH} and Ct_{β-Actin}. Statistical inference tests performed on the ΔCt values were Ordinary one-way ANOVA (p-values are indicated in panel D), with multiple comparison test according to two-stage linear step-up procedure of Benjamini, Krieger and Yekutieli (Benjamini et al., 2006). False discovery rate (FDR)<0.05 was considered to be statistically significant. cDNA samples obtained from known human primary cells and cell lines were employed as positive control for each qRT-PCR experiment (Supplemental figure S10). In this regard, the human leukemia cell line KG1a was used as positive control for MGAT1, GCNT1, B4GALT1, SLC35C1, ST3GAL3, ST3GAL4, ST3GAL6, NEU1, NEU3, FUT4, and FUT7 genes. Human bone marrow mesenchymal stem cells (BM-MSCs) were used as positive control for FUC1 and FUC2 genes. Sublines of the human prostate cancer cell line PCR1, containing stable cDNA constructs encoding either FUT3, FUT6, or FUT7, were used as positive control for the respective genes. Human peripheral blood neutrophil and human bone marrow buffy coat (BM-Buffy coat) were used as positive control for FUT9 gene expression.

### Exofucosylation

Exofucosylation was performed by incubating 1×10⁷ cells/mi cells with reaction mixture containing 1 mM GDP-fucose and either 60 μg/ml purified FT6 enzyme (Dykstra et al., 2016), or 100 μg/ml purified FT9 enzyme (Bio-techne, Minneapolis, MN) in Hank's Balanced Salt Solution (HBSS) at 37° C. for 1 hr.

### In Vitro Hematopoietic Colony Forming Assay

Hematopoietic colony forming assay was performed using Methocult 4434 media (STEMCELL technologies) according to manufacturer's protocol. Briefly, appropriately isolated HSPC subpopulations were resuspended in Methocult media and dispensed into a 3.5 mm tissue culture dish and incubated at 37° C. with 5% CO₂ for 7-14 days. Hematopoietic colonies were evaluated based on colony morphology (Wognum et al., 2013) after 14 days of culture.

### Xenotransplantation Assay to Measure Bone Marrow Homing of HSPCs

5×10⁵ CD34⁺ human HSPCs (either BT or FT6 exofucosylated) were stained with either CellTrace™ CFSE (Thermo Fisher Scientific) or SNARF-1 (Thermo Fisher Scientific) as described in (Dykstra et al., 2016). CFSE and SNARF-1 labeled cells were mixed in 1:1 ratio and injected in the retro-orbital plexus of NOD-scid IL2rgtm1Wjl/Sz (NSG) mice (Jackson laboratories). In one experimental combination, CFSE-labeled buffer-treated (BT) cells were mixed in equal proportions with SNARF-1-labeled FT6 exofucosylated cells. In another experimental combination, CFSE-labeled FT6-treated cells were mixed with SNARF-1-labeled buffer-treated cells. For control combinations, CFSE- and SNARF-1-labeled buffer-treated cells were mixed in equal proportions. For the last control combination, CFSE and SNARF-1 labeled FT6-treated mixed in equal proportions. 16 hours post transplantation, bone marrow was harvested from both femurs and both tibias of each mouse (representing ˜18.9% of total bone marrow of the mouse (Boggs, 1984)). CFSE and SNARF-1 labeled cells within the marrow cells were measured by flow cytometry, and the ratios of CFSE- and SNARF-1-labeled cells were calculated for each and presented as the fold-difference in homed cells between BT and FT6 HSPCs.

### In Vivo Xenotransplantation Assay to Assess Short-Term Engraftment of Human Hematopoietic Cells

5×10⁴ cells from either buffer-treated or FT6-exofucosylated groups of CD34⁺CD38⁺ UCB-HSPCs were injected into the retro-orbital plexus of sub-lethally irradiated (234 cGy) NSG mice (Jackson laboratories). Neomycin solution was added to the drinking water for two weeks after irradiation. As an additional control, 5,000 untreated CD34⁺CD38− HSPCs were injected. Blood was collected every three days post-transplantation via tail bleeding and analyzed using flow cytometry after staining with antibodies against mouse CD45 and human CD45. Human chimerism in mouse blood was measured by quantifying the number of anti-mouse CD45 negative, and, anti-human CD45-PE and anti-human CD45-APC-Cy7 double positive cells (Supplemental Figure S11).

### Xenotransplantation Assay to Measure Long-Term Engraftment of Human Hematopoietic Cells

Three HSPC sub-populations were isolated based on cell surface sLeX expression, according to the following three phenotypes: P1=CD34⁺CD38⁻sLeX^{high} (highest 10% fraction of sLeX-staining MFI); P2=CD34⁺CD38⁻sLeX^{low/-} (lowest 10% fraction of sLeX-staining MFI); and P3=CD34+sLeX^{high} For control experiments, isolated HSC_{conv}s were used. 500, 1000, or 2000 of each group (i.e., P1, P2, P3, HSC_{conv}s) were then suspended in 100 μL 1×PBS and injected into the retro-orbital plexus of sub-lethally irradiated (225 cGy) NSG mice. Post-xenotransplant, mouse blood was collected via tail bleeding at several time points until 24 weeks from the day of injection. Leukocytes obtained after lysis of red blood cells were then stained with antibodies against mouse CD45 (APC-conjugated) and against human CD45 (APCCy7- and FITC-conjugated) and analyzed using flow cytometry. Human chimerism was evaluated by enumerating APC⁻ and APCCy7 and FITC double positive cells (Supplemental figure S12).

cDNA Library Preparation and RNA Sequencing

cDNA was synthesized from the total cellular RNA using Clontech SmartSeq v4 reagents from 1 ng of RNA. Full length cDNA was fragmented to a mean size of 150 bp with a Covaris M220 ultrasonicator and Illumina libraries were prepared from 2 ng of sheared cDNA using Rubicon Genomics Thruplex DNAseq reagents according to manufacturer's protocol. The finished dsDNA libraries were quantified by Qubit fluorometer, Agilent TapeStation 2200, and RT-qPCR using the Kapa Biosystems library quantification kit. Uniquely indexed libraries were pooled in equimolar ratios and sequenced on an Illumina NextSeq500 run with single-end 75 bp reads at the Dana-Farber Cancer Institute Molecular Biology Core Facilities.

### Bioinformatics Analysis

RNA reads were subjected to multiQC programs to quantify overall quality of the sequencing. The RNA reads were then aligned with reference human genome version 38 (GRCh38) using the alignment program Hisat2 (Kim et al., 2015). The resulting SAM files were then converted to BAM format using SAMtoBAM conversion tool (Johns Hopkins University, open access). All downstream statistical analyses were performed on the SeqMonk analysis software (Babraham Bioinformatics, Cambridge UK) unless otherwise mentioned. Genes having differential expression among the isolated subpopulations were identified using the differential expression analysis algorithm on the aligned raw count matrix (Love et al., 2014). Principal component analysis was performed on the differentially expressed genes to identify the transcriptional relationships between the isolated subpopulations. Transcriptional similarity within subpopulations were estimated using unsupervised hierarchical clustering and by calculating Pearson's correlation coefficients. To estimate the distance between individual subpopulations, the number of genes with significantly different expression between each pair of subsets were evaluated by performing moderated T-test with Benjamini-Hochberg multiple testing correction (62) using the R based LIMMA (Linear Models of Microarray Analysis) package on SeqMonk. For each subset pair, the set of differentially expressed genes were subjected to DAVID (The Database for Annotation, Visualization and Integrated Discovery) analysis to identify gene ontology (GO) categories overrepresented by the gene sets tested. The gene sets over-representing each GO category were mined from DAVID analysis, and their expression levels were compared within the tested subpopulations.

The embodiments described in this disclosure can be combined in various ways. Any aspect or feature that is described for one embodiment can be incorporated into any other embodiment mentioned in this disclosure. While various novel features of the inventive principles have been shown, described and pointed out as applied to particular embodiments thereof, it should be understood that various omissions and substitutions and changes can be made by those skilled in the art without departing from the spirit of this disclosure. Those skilled in the art will appreciate that the inventive principles can be practiced in other than the described embodiments, which are presented for purposes of illustration and not limitation.

### Various Embodiments

Embodiment 1. A method for selecting one or more human hematopoietic stem/progenitor cells (HSPCs) from within a heterogenous population of lin⁻ HSPCs comprising:
contacting the heterogenous population of lin⁻ HSPCs with a binding molecule for sialylated Lewis X (sLeX);
measuring the amount of sLeX present on individual cells in the heterogenous population of lin⁻ HSPCs; and
selecting for one or more of sLeX^{high} cells based on the level of sLeX expression of the lin⁻ HSPCs, wherein the sLeX^{high} cells are the cells having the highest 15% sLeX expression level within the heterogenous population of sLeX+lin⁻ HSPCs.

Embodiment 2. The method according to any preceding Embodiment, further comprising the step of selecting for CD38− cells.

Embodiment 3. The method according to any preceding Embodiment, further comprising the step of selecting for CD34+ cells.

Embodiment 4. The method according to any preceding Embodiment, further comprising the step of selecting for CD38− cells.

Embodiment 5. The method according to any preceding Embodiment, further comprising the step of selecting for CD38+ cells.

Embodiment 6. The method according to any preceding Embodiment, further comprising the step of selecting for CD34+ cells.

Embodiment 7. The method according to any preceding Embodiment, wherein the heterogenous population of lin⁻ HSPCs is from bone marrow, umbilical cord blood, adult (post-natal) blood, fetal blood, fetal liver, fetal spleen, embryonic yolk sac, embryonic ventral endothelium of dorsal aorta, adult (post-natal) liver, or adult (post-natal) spleen.

Embodiment 8. The method according to any preceding Embodiment, wherein the heterogenous population of lin⁻ HSPCs are obtained by one or more steps of depleting differentiated HSPCs expressing lineage markers (i.e., depletion of lin+ nucleated cells) Embodiment 9. The method according to any preceding Embodiment, wherein the selection for one or more of sLeX^{high} and CD38− cells comprises one or more steps of positive selection or negative selection.

Embodiment 10. The method according to any preceding Embodiment, wherein the selection of sLeX^{high} cells comprises one or more negative selection steps for depleting cells within the heterogenous population of sLeX+lin⁻ human HSPCs that express sLeX at density levels within the lower 85% of the range of sLeX expression within the heterogenous sLeX+lin⁻ cell population.

Embodiment 11. The method according to any preceding Embodiment, wherein the selecting for one or more of sLeX^{high} cells comprises selecting for cells having the highest 10% of sLeX expression level within the heterogenous population of sLeX+lin⁻ cells.

Embodiment 12. The method according to any preceding Embodiment, wherein the selecting step comprises use of a molecule that binds the glycan determinant sLeX and the anti-determinant molecule contains a selection tag whereby cells bearing the anti-determinant molecule (e.g., anti-sLeX antibody, E-selectin-Ig chimera, etc.) can then be separated.

Embodiment 13. The method according to any preceding Embodiment, wherein the selecting step comprises use of molecule that binds the glycan determinant sLeX that carries a functional group to allow detection and separation of cells bearing the molecule-functional group, such as the use of magnetic bead-tagged anti-determinant molecules (e.g., magnetic bead-conjugated anti-sLeX antibody, magnetic bead-conjugated E-selectin-Ig chimera), biotin-tagged anti-determinant molecules (e.g., biotin-tagged anti-sLeX antibody, biotin-tagged E-selectin-Ig chimera), fluorescence-activated cell sorting (FACS) utilizing fluorochrome-tagged anti-determinant molecules (e.g., direct (one-step) using a fluorochrome-tagged anti-determinant molecules, or indirect (two-step) using a fluorochome-tagged secondary reagent that recognizes the (primary) anti-determinant molecule (indirect (two-step) fluorochrome labelling of the cell)), chemically-modified anti-determinant molecules (e.g., anti-determinant molecule modified to contain a “clickable” chemical reagent such as an alkyne or azide modification), “panning” by affixing the anti-determinant molecules to a solid support matrix, passage of cells over affinity columns containing the anti-determinant molecules attached to beads or other matrices, or other techniques providing accurate cell separation.

Embodiment 14. A method for selecting one or more human hematopoietic stem/progenitor cells (HSPCs) from within a heterogenous population of lin⁻ HSPCs comprising:
contacting the heterogenous population of lin⁻ HSPCs with a binding molecule for sialylated Lewis X (sLeX);
measuring the amount of sLeX present on individual cells in the heterogenous population of lin⁻ HSPCs; and
selecting for one or more of sLeX^{low/-} cells based on the level of sLeX expression of the cells, wherein the sLeX^{low/-} subset comprises a fraction of 15% of the heterogenous population whose composition ranges from cells that lack sLeX expression to cells with the lowest level expression of sLeX (i.e., those cells that by FACS comprise the 15% fraction of the entire population that have the lowest fluorescence staining level for sLeX).

Embodiment 15. The method according to any preceding Embodiment, further comprising the step of selecting for CD38− cells.

Embodiment 16. The method according to any preceding Embodiment, further comprising the step of selecting for CD34+ cells.

Embodiment 17. The method according to any preceding Embodiment, further comprising the step of selecting for CD38− cells.

Embodiment 18. The method according to any preceding Embodiment, further comprising the step of selecting for CD38+ cells.

Embodiment 19. The method according to any preceding Embodiment, further comprising the step of selecting for CD34+ cells.

Embodiment 20. The method according to any preceding Embodiment, wherein the heterogenous population of lin⁻ HSPCs is from bone marrow, umbilical cord blood, adult (post-natal) blood, fetal blood, fetal liver, fetal spleen, embryonic yolk sac, embryonic ventral endothelium of dorsal aorta, adult (post-natal) liver, or adult (post-natal) spleen.

Embodiment 21. The method according to any preceding Embodiment, wherein the selection for one or more of sLeX^{low/-}, CD34+, and CD38− cells comprises one or more steps of positive selection or negative selection to select for cells expressing the markers sLeX^{low/-}, CD34 and CD38.

Embodiment 22. The method according to any preceding Embodiment, wherein the heterogenous population of lin⁻ HSPCs are obtained by one or more steps of depleting differentiated HSPCs expressing lineage markers (i.e., depletion of lin+ cells).

Embodiment 23. The method according to any preceding Embodiment, wherein the selection comprises one or more negative selection steps to enrich a population of sLeX^{low/-} cells by depletion of cells expressing sLeX at cell density levels >85% of the level within the heterogenous cell population.

Embodiment 24. The method according to any preceding Embodiment, wherein the sLeX^{low/-} cells comprises a fraction of 10% of the heterogenous population whose composition ranges from cells that lack sLeX expression to cells with the lowest level expression of sLeX.

Embodiment 25. The method according to any preceding Embodiment, wherein the selecting step comprises use of a molecule that binds the determinant (e.g., sLeX) and the anti-determinant molecule contains a selection tag whereby cells bearing the anti-determinant molecule (e.g., anti-sLeX antibody, E-selectin-Ig chimera, etc.) can then be separated.

Embodiment 26. The method according to any preceding Embodiment, wherein the selecting step comprises magnetic bead-tagged anti-determinant molecules (e.g., magnetic bead-conjugated anti-sLeX antibody, magnetic bead-conjugated E-selectin-Ig chimera), biotin-tagged anti-determinant molecules (e.g., biotin-tagged anti-sLeX antibody, biotin-tagged E-selectin-Ig chimera), fluorescence-activated cell sorting (FACS) utilizing fluorochrome-tagged anti-determinant molecules (e.g., direct (one-step) using a fluorochrome-tagged anti-determinant molecule, or indirect (two-step) using a fluorochome-tagged secondary reagent that recognizes the (primary) anti-determinant molecules (indirect (two-step) fluorochrome labelling of the cell)), chemically-modified anti-determinant molecules (e.g., anti-determinant molecule modified to contain a “clickable” chemical reagent such as an alkyne or azide modification), “panning” by affixing the anti-determinant molecules to a solid support matrix, passage of cells over affinity columns containing the anti-determinant molecules attached to beads or other matrices, or other techniques providing accurate cell separation.

Embodiment 27. A method for grading the level of expression of terminal α(2,3)-sialylated Type-2 lactosamine units on cells comprising the steps of:
(a) measuring the level of expression of sialylated Lewis X (sLeX) on the surface of one or more cells;
(b) contacting the one or more cells with an α(1,3)-fucosyltransferase capable of creation of sLeX from an acceptor terminal α(2,3)-sialylated Type-2 lactosamine together with a nucleotide donor sugar (GDP-fucose);
(c) measuring the level of sLeX expression on the fucosyltransferase-treated cells, wherein the increase in sLeX expression from Step (a) compared to that following Step (b) indicates the level of terminal α(2,3)-sialylated Type-2 lactosamine units on the one or more cells.

Embodiment 28. The method according to any preceding Embodiment, wherein the α(1,3)-fucosyltransferase is selected from the group consisting of FTVI, FTVII, FTIII, FTV, and FTIV.

Embodiment 29. The method according to any preceding Embodiment, wherein the GDP-fucose is modified with a selection tag that allows for separation of those cells containing the installed fucose.

Embodiment 30. The method according to any preceding Embodiment, wherein the selection tag consists of a chemically “tagged” GDP-fucose covalently modified with a fluorochrome, a “clickable” chemical group, biotin, a radiolabel, or any other molecule covalently linked to the fucose moiety within GDP-fucose that can be used to identify the installed fucose.

Embodiment 31. The method according to any preceding Embodiment, wherein the measuring of step (a) and step (b) comprises contacting the sLeX with a fluorescent binder and measuring mean fluorescence intensity (MFI) by flow cytometry.

Embodiment 32. A method for grading the level of expression of terminal “neutral” Type-2 lactosamine units on cells comprising the steps of:
(a) measuring the level of expression of Lewis X (LeX) on the surface of one or more cells;
(b) contacting the one or more cells with an α(1,3)-fucosyltransferase capable of creating LeX from an acceptor terminal “neutral” Type-2 lactosamine together with a nucleotide donor sugar (GDP-fucose);
(c) measuring the level of LeX expression on the fucosyltransferase-treated cells, wherein the increase in LeX level expression from Step (a) compared to that following Step (b) indicates the level of terminal “neutral” Type-2 lactosamine units on the one or more cells.

Embodiment 33. The method according to any preceding Embodiment, wherein the α(1,3)-fucosyltransferase is selected from the group consisting of FTIX, FTVI, FTIV, FTIII, and FTV.

Embodiment 34. The method according to any preceding Embodiment, wherein the GDP-fucose is modified with a chemical tag that allows for separation of those cells containing the installed fucose.

Embodiment 35. The method according to any preceding Embodiment, wherein the selection tag consists of a chemically “tagged” GDP-fucose covalently modified with a fluorochrome, a “clickable” chemical group, biotin, a radiolabel, or any other molecule covalently linked to the fucose moiety within GDP-fucose that can be used to identify the installed fucose.

Embodiment 36. The method according to any preceding Embodiment, wherein the measuring of step (a) and step (b) comprises contacting the LeX with a fluorescent binder and measuring mean fluorescence intensity (MFI) by flow cytometry.

Embodiment 37. A method of selecting cells having free terminal α(2,3)-sialylated Type-2 lactosamine units comprising the steps of:
(a) contacting a population of cells with an α(1,3)-fucosyltransferase capable of creation of sLeX from an acceptor terminal α(2,3)-sialylated Type-2 lactosamine together with a chemically-tagged nucleotide donor sugar (tagged GDP-fucose); and
(b) selecting for cells having sLeX comprising the tagged-fucose within the population of cells;
wherein the selected cells from step (b) comprise cells originally having free terminal α(2,3)-sialylated Type-2 lactosamine units.

Embodiment 38. The method according to any preceding Embodiment wherein the α(1,3)-fucosyltransferase is selected from the group consisting of FTVI, FTVII, FTIII, FTV, and FTIV.

Embodiment 39. The method according to any preceding Embodiment, wherein the chemical tag is selected from the group consisting of a fluorochrome, a clickable chemical group, biotin, a radiolabel, or any other molecule covalently linked to the fucose moiety within GDP-fucose that can be used to identify the installed fucose.

Embodiment 40. A method of selecting cells having terminal free “neutral” Type-2 lactosamine units comprising the steps of
(a) contacting a population of cells with an α(1,3)-fucosyltransferase capable of creation of LeX from an acceptor terminal “neutral” Type-2 lactosamine together with a tagged nucleotide donor sugar (tagged GDP-fucose);
(b) selecting for cells having LeX comprising the installed tagged fucose within the population of cells;
wherein the selected cells from step (b) comprise cells originally having terminal free Type-2 lactosamine units.

Embodiment 42. The method according to any preceding Embodiment wherein the α(1,3)-fucosyltransferase is selected from the group consisting of FTIX, FTVI, FTIV, FTIII, and FTV.

Embodiment 43. The method according to any preceding Embodiment, wherein the chemical tag is selected from the group consisting of a “tagged” GDP-fucose covalently modified with a fluorochrome, a clickable group, biotin, a radiolabel, or any other molecule covalently linked to the fucose moiety within GDP-fucose that can be used to identify the installed fucose.

Embodiment 44. A composition comprising the cells selected according to any according to any preceding Embodiment.

Embodiment 45. A method of treating a human subject in need thereof comprising the step of administering to the human subject a therapeutically effective amount of the cells selected according to according to any preceding Embodiment

Embodiment 46. The method according to any preceding Embodiment, wherein the cells are effective to treat one or more of hematologic diseases/disorders/conditions, genetic diseases/disorders/conditions, congenital diseases/disorders/conditions, degenerative diseases/disorders/conditions, cancerous diseases/disorders/conditions, immune diseases/disorders/conditions, drug reactions, toxin-induced injury, psychiatric diseases/disorders/conditions, vascular diseases/disorders/conditions, inflammatory diseases/disorders/conditions, iatrogenic conditions, infectious diseases/disorders/conditions, trauma, burns, ischemia/reperfusion injury, nervous system diseases/disorders/conditions, sepsis, cytokine-induced diseases/conditions/disorders, and tissue/organ failure.

Embodiment 47. A kit for enriching or isolating one or more human hematopoietic stem/progenitor cells (HSPCs) from within a heterogenous population of HSPCs comprising reagents for selecting one or more of the markers sLeX, CD38, and CD34, and instructions for use thereof.

### REFERENCES

The following references, to the extent that they provide exemplary procedural or other details supplementary to those set forth herein, are specifically incorporated herein by reference.
Akashi, K., Traver, D., Miyamoto, T., and Weissman, I. L. (2000). A clonogenic common myeloid progenitor that gives rise to all myeloid lineages. Nature 404, 193. 10.1038/35004599.
Al-Amoodi, A. S., Li, Y., Al-Ghuneim, A., Allehaibi, H., Isaioglou, I., Esau, L. E., AbuSamra, D. B., and Merzaban, J. S. (2022). Refining the migration and engraftment of short-term and long-term HSCs by enhancing homing-specific adhesion mechanisms. Blood Adv 6, 4373-4391. 10.1182/bloodadvances.2022007465.
Benjamini, Y., Krieger, A. M., and Yekutieli, D. (2006). Adaptive linear step-up procedures that control the false discovery rate. Biometrika 93, 491-507. 10.1093/biomet/93.3.491.
Boggs, D. R. (1984). The total marrow mass of the mouse: A simplified method of measurement. American Journal of Hematology 16, 277-286. 10.1002/ajh.2830160309.
Buffone, A., Jr., Mondal, N., Gupta, R., McHugh, K. P., Lau, J. T., and Neelamegham, S. (2013). Silencing alphal,3-fucosyltransferases in human leukocytes reveals a role for FUT9 enzyme during E-selectin-mediated cell adhesion. The Journal of biological chemistry 288, 1620-1633. 10.1074/jbc.M112.400929.
Butcher, E. C. (1991). Leukocyte-endothelial cell recognition: three (or more) steps to specificity and diversity. Cell 67, 1033-1036. 10.1016/0092-8674(91)90279-8.
Copley, Michael R., Beer, Philip A., and Eaves, Connie J. (2012). Hematopoietic Stem Cell Heterogeneity Takes Center Stage. Cell Stem Cell 10, 690-697. https://doi.org/10.1016/j.stem.2012.05.006.
Dagia, N. M., Gadhoum, S. Z., Knoblauch, C. A., Spencer, J. A., Zamiri, P., Lin, C. P., and Sackstein, R. (2006). G-CSF induces E-selectin ligand expression on human myeloid cells. Nature medicine 12, 1185-1190. 10.1038/nm1470.
Dimitroff, C. J., Lee, J. Y., Rafii, S., Fuhlbrigge, R. C., and Sackstein, R. (2001). CD44 is a major E-selectin ligand on human hematopoietic progenitor cells. J Cell Biol 153, 1277-1286.
Doulatov, S., Notta, F., Eppert, K., Nguyen, L. T., Ohashi, P. S., and Dick, J. E. (2010). Revised map of the human progenitor hierarchy shows the origin of macrophages and dendritic cells in early lymphoid development. Nature Immunology 11, 585. 10.1038/ni.1889; https://www.nature.com/articles/ni.1889 #supplementary-information.
Dykstra, B., Lee, J., Mortensen, L. J., Yu, H., Wu, Z. L., Lin, C. P., Rossi, D. J., and Sackstein, R. (2016). Glycoengineering of E-Selectin Ligands by Intracellular versus Extracellular Fucosylation Differentially Affects Osteotropism of Human Mesenchymal Stem Cells. STEM CELLS 34, 2501-2511. 10.1002/stem.2435.
Enver, T., Pera, M., Peterson, C., and Andrews, P. W. (2009). Stem cell states, fates, and the rules of attraction. Cell Stem Cell 4, 387-397. 10.1016/j.stem.2009.04.011.
Eppert, K., Takenaka, K., Lechman, E. R., Waldron, L., Nilsson, B., van Galen, P., Metzeler, K. H., Poeppl, A., Ling, V., Beyene, J., et al. (2011). Stem cell gene expression programs influence clinical outcome in human leukemia. Nature medicine 17, 1086. 10.1038/nm.2415https://www.nature.com/articles/nm.2415 #supplementary-information.
Frenette, P. S., Subbarao, S., Mazo, I. B., von Andrian, U. H., and Wagner, D. D. (1998). Endothelial selectins and vascular cell adhesion molecule-1 promote hematopoietic progenitor homing to bone marrow. Proceedings of the National Academy of Sciences 95, 14423-14428. 10.1073/pnas.95.24.14423.
Gadhoum, S. Z., and Sackstein, R. (2008). CD15 expression in human myeloid cell differentiation is regulated by sialidase activity. Nat Chem Biol 4, 751-757. 10.1038/nchembio.116.
Georgantas, R. W., Tanadve, V., Malehom, M., Heimfeld, S., Chen, C., Carr, L., Martinez-Murillo, F., Riggins, G., Kowalski, J., and Civin, C. I. (2004). Microarray and Serial Analysis of Gene Expression Analyses Identify Known and Novel Transcripts Overexpressed in Hematopoietic Stem Cells. Cancer Research 64, 4434-4441. 10.1158/0008-5472.can-03-3247.
Gooi, H. C., Thorpe, S. J., Hounsell, E. F., Rumpold, H., Kraft, D., Forster, O., and Feizi, T. (1983). Marker of peripheral blood granulocytes and monocytes of man recognized by two monoclonal antibodies VEP8 and VEP9 involves the trisaccharide 3-fucosyl-N-acetyllactosamine. Eur J Immunol 13, 306-312. 10.1002/eji.1830130407.
Griffin, M. E., and Hsieh-Wilson, L. C. (2016). Glycan Engineering for Cell and Developmental Biology. Cell Chem Biol 23, 108-121. 10.1016/j.chembiol.2015.12.007.
Handa, K., Stroud, M. R., and Hakomori, S.-i. (1997). Sialosyl-Fucosyl Poly-LacNAc without the Sialosyl-Lex Epitope as the Physiological Myeloid Cell Ligand in E-Selectin-Dependent Adhesion: Studies under Static and Dynamic Flow Conditions. Biochemistry 36, 12412-12420. 10.1021/bi971181t.
Hidalgo, A., and Frenette, P. S. (2005). Enforced fucosylation of neonatal CD34+ cells generates selectin ligands that enhance the initial interactions with microvessels but not homing to bone marrow. Blood 105, 567-575. 10.1182/blood-2004-03-1026.
Homeister, J. W., Thall, A. D., Petryniak, B., Malý, P., Rogers, C. E., Smith, P. L., Kelly, R. J., Gersten, K. M., Askari, S. W., Cheng, G., et al. (2001). The α(1,3)fucosyltransferases FucT-IV and FucT-VII Exert Collaborative Control over Selectin-Dependent Leukocyte Recruitment and Lymphocyte Homing. Immunity 15, 115-126. https://doi.org/10.1016/S1074-7613(01)00166-2.
Huang, D. W., Sherman, B. T., and Lempicki, R. A. (2008). Systematic and integrative analysis of large gene lists using DAVID bioinformatics resources. Nature Protocols 4, 44. 10.1038/nprot.2008.211
https://www.nature.com/articles/nprot.2008.211 #supplementary-information.
Huang, D. W., Sherman, B. T., and Lempicki, R. A. (2009). Bioinformatics enrichment tools: paths toward the comprehensive functional analysis of large gene lists. Nucleic acids research 37, 1-13. 10.1093/nar/gkn923.
Katayama, Y., Hidalgo, A., Furie, B. C., Vestweber, D., Furie, B., and Frenette, P. S. (2003). PSGL-1 participates in E-selectin-mediated progenitor homing to bone marrow: evidence for cooperation between E-selectin ligands and α4 integrin. Blood 102, 2060-2067. 10.1182/blood-2003-04-1212.
Kim, D., Langmead, B., and Salzberg, S. L. (2015). HISAT: a fast spliced aligner with low memory requirements. Nature Methods 12, 357. 10.1038/nmeth.3317
https://www.nature.com/articles/nmeth.3317 #supplementary-information.
Laird, D. J., von Andrian, U. H., and Wagers, A. J. (2008). Stem cell trafficking in tissue development, growth, and disease. Cell 132, 612-630. 10.1016/j.cell.2008.01.041.
Lanctot, P. M., Gage, F. H., and Varki, A. P. (2007). The glycans of stem cells. Current opinion in chemical biology 11, 373-380. 10.1016/j.cbpa.2007.05.032.
Lapidot, T., Dar, A., and Kollet, O. (2005). How do stem cells find their way home? Blood 106, 1901-1910. 10.1182/blood-2005-04-1417.
Love, M. I., Huber, W., and Anders, S. (2014). Moderated estimation of fold change and dispersion for RNA-seq data with DESeq2. Genome Biology 15, 550. 10.1186/s13059-014-0550-8.
Majeti, R., Park, C. Y., and Weissman, I. L. (2007). Identification of a Hierarchy of Multipotent Hematopoietic Progenitors in Human Cord Blood. Cell Stem Cell 1, 635-645. https://doi.org/10.1016/j.stem.2007.10.001.
Manz, M. G., Miyamoto, T., Akashi, K., and Weissman, I. L. (2002). Prospective isolation of human clonogenic common myeloid progenitors. Proceedings of the National Academy of Sciences 99, 11872-11877. 10.1073/pnas.172384399.
Mayani, H., Dragowska, W., and Lansdorp, P. (1993). Characterization of functionally distinct subpopulations of CD34+ cord blood cells in serum-free long-term cultures supplemented with hematopoietic cytokines. Blood 82, 2664-2672.
Mazo, I. B., Gutierrez-Ramos, J.-C., Frenette, P. S., Hynes, R. O., Wagner, D. D., and von Andrian, U. H. (1998). Hematopoietic Progenitor Cell Rolling in Bone Marrow Microvessels: Parallel Contributions by Endothelial Selectins and Vascular Cell Adhesion Molecule 1. The Journal of Experimental Medicine 188, 465-474. 10.1084/jem.188.3.465.
Merzaban, J. S., Burdick, M. M., Gadhoum, S. Z., Dagia, N. M., Chu, J. T., Fuhlbrigge, R. C., and Sackstein, R. (2011). Analysis of glycoprotein E-selectin ligands on human and mouse marrow cells enriched for hematopoietic stem/progenitor cells. Blood 118, 1774-1783. 10.1182/blood-2010-11-320705.
Miller, P. H., Knapp, D. J., and Eaves, C. J. (2013). Heterogeneity in hematopoietic stem cell populations: implications for transplantation. Current opinion in hematology 20, 257-264. 10.1097/MOH.0b013e328360aaf6.
Mondal, N., Buffone, A., Stolfa, G., Antonopoulos, A., Lau, J. T. Y., Haslam, S. M., Dell, A., and Neelamegham, S. (2015). ST3Gal-4 is the primary sialyltransferase regulating the synthesis of E-, P-, and L-selectin ligands on human myeloid leukocytes. Blood 125, 687-696. 10.1182/blood-2014-07-588590.
Mondal, N., Dykstra, B., Lee, J., Ashline, D. J., Reinhold, V. N., Rossi, D. J., and Sackstein, R. (2018). Distinct human α(1,3)-fucosyltransferases drive Lewis-X/sialyl Lewis-X assembly in human cells. Journal of Biological Chemistry 293, 7300-7314. 10.1074/jbc.RA117.000775.
Mondal, N., Stolfa, G., Antonopoulos, A., Zhu, Y., Wang, S.-S., Buffone, A., Atilla-Gokcumen, G. E., Haslam, S. M., Dell, A., and Neelamegham, S. (2016). Glycosphingolipids on Human Myeloid Cells Stabilize E-Selectin-Dependent Rolling in the Multistep Leukocyte Adhesion Cascade. Arteriosclerosis, Thrombosis, and Vascular Biology 36, 718-727. 10.1161/atvbaha.115.306748.
Muramatsu, T., and Muramatsu, H. (2004). Carbohydrate antigens expressed on stem cells and early embryonic cells. Glycoconjugate journal 21, 41-45. 10.1023/B:GLYC.0000043746.77504.28.
Nielsen, J. S., and McNagny, K. M. (2008). Novel functions of the CD34 family. Journal of Cell Science 121, 3683-3692. 10.1242/jcs.037507.
Nimrichter, L., Burdick, M. M., Aoki, K., Laroy, W., Fierro, M. A., Hudson, S. A., Von Seggem, C. E., Cotter, R. J., Bochner, B. S., Tiemeyer, M., et al. (2008). E-selectin receptors on human leukocytes. Blood 112, 3744-3752. 10.1182/blood-2008-04-149641.
Nonomura, C., Ando, H., Shinohara, H., Nojiri, H., Kikuchi, J., Nakamura, M., and Takaku, S. (2004). Not core 2 β1,6—N-acetylglucosaminyltransferase-2 or -3 but-1 regulates sialyl-Lewis x expression in human precursor B cells. Glycobiology 15, 271-280. 10.1093/glycob/cwi005.
Notta, F., Doulatov, S., Laurenti, E., Poeppl, A., Jurisica, I., and Dick, J. E. (2011). Isolation of Single Human Hematopoietic Stem Cells Capable of Long-Term Multilineage Engraftment. Science 333, 218-221. 10.1126/science.1201219.
Olweus, J., Lund-Johansen, F., and Terstappen, L. W. M. M. (1994). Expression of Cell Surface Markers during Differentiation of CD34+, CD38−/lo Fetal and Adult Bone Marrow Cells. ImmunoMethods 5, 179-188. https://doi.org/10.1006/immu.1994.1054.
Pachón-Peña, G., Donnelly, C., Ruiz-Cañada, C., Katz, A., Fernández-Veledo, S., Vendrell, J., and Sackstein, R. (2017). A Glycovariant of Human CD44 is Characteristically Expressed on Human Mesenchymal Stem Cells. Stem cells (Dayton, Ohio) 35, 1080-1092. 10.1002/stem.2549.
Peled, A., Grabovsky, V., Habler, L., Sandbank, J., Arenzana-Seisdedos, F., Petit, I., Ben-Hur, H., Lapidot, T., and Alon, R. (1999). The chemokine SDF-1 stimulates integrin-mediated arrest of CD34⁺ cells on vascular endothelium under shear flow. The Journal of Clinical Investigation 104, 1199-1211. 10.1172/JC17615.
Peled, A., Kollet, O., Ponomaryov, T., Petit, I., Franitza, S., Grabovsky, V., Slav, M. M., Nagler, A., Lider, O., Alon, R., et al. (2000). The chemokine SDF-1 activates the integrins LFA-1, VLA-4, and VLA-5 on immature human CD34+ cells: role in transendothelial/stromal migration and engraftment of NOD/SCID mice. Blood 95, 3289-3296.
Popat, U., Mehta, R. S., Rezvani, K., Fox, P., Kondo, K., Marin, D., McNiece, I., Oran, B., Hosing, C., Olson, A., et al. (2015). Enforced fucosylation of cord blood hematopoietic cells accelerates neutrophil and platelet engraftment after transplantation. Blood 125, 2885-2892. 10.1182/blood-2015-01-607366.
Prescher, J. A., and Bertozzi, C. R. (2006). Chemical technologies for probing glycans. Cell 126, 851-854. 10.1016/j.cell.2006.08.017.
Robinson, S. N., Simmons, P. J., Thomas, M. W., Brouard, N., Javni, J. A., Trilok, S., Shim, J.-S., Yang, H., Steiner, D., Decker, W. K., et al. (2012). Ex vivo fucosylation improves human cord blood engraftment in NOD-SCID IL-2Rγnull mice. Experimental Hematology 40, 445-456. https://doi.org/10.1016/j.exphem.2012.01.015.
Robinson, S. N., Thomas, M. W., Simmons, P. J., Lu, J., Yang, H., Parmar, S., Liu, X., Shah, N., Martin-Antonio, B., Bollard, C., et al. (2014). Fucosylation with fucosyltransferase VI or fucosyltransferase VII improves cord blood engraftment. Cytotherapy 16, 84-89. 10.1016/j.jcyt.2013.07.003.
Sackstein, R. (2004). The bone marrow is akin to skin: HCELL and the biology of hematopoietic stem cell homing. J Investig Dermatol 122, 1061-1069. 10.1111/j.0022-202X.2004.09301.x.
Sackstein, R. (2009). Glycosyltransferase-programmed stereosubstitution (GPS) to create HCELL: engineering a roadmap for cell migration. Immunological Reviews 230, 51-74. doi:10.1111/j.1600-065X.2009.00792.x.
Sackstein, R. (2016). Fulfilling Koch's postulates in glycoscience: HCELL, GPS and translational glycobiology. Glycobiology 26, 560-570. 10.1093/glycob/cww026.
Sackstein, R., Merzaban, J. S., Cain, D. W., Dagia, N. M., Spencer, J. A., Lin, C. P., and Wohlgemuth, R. (2008). Ex vivo glycan engineering of CD44 programs human multipotent mesenchymal stromal cell trafficking to bone. Nature medicine 14, 181-187. 10.1038/nm1703.
Sanada, C., Xavier-Ferrucio, J., Lu, Y.-C., Min, E., Zhang, P.-X., Zou, S., Kang, E., Zhang, M., Zerafati, G., Gallagher, P. G., and Krause, D. S. (2016). Adult human megakaryocyte-erythroid progenitors are in the CD34+CD38mid fraction. Blood 128, 923-933. 10.1182/blood-2016-01-693705.
Schopperle, W. M., and DeWolf, W. C. (2007). The TRA-1-60 and TRA-1-81 human pluripotent stem cell markers are expressed on podocalyxin in embryonal carcinoma. Stem cells (Dayton, Ohio) 25, 723-730. 10.1634/stemcells.2005-0597.
Schweitzer, K. M., Drager, A. M., van der Valk, P., Thijsen, S. F., Zevenbergen, A., Theijsmeijer, A. P., van der Schoot, C. E., and Langenhuijsen, M. M. (1996). Constitutive expression of E-selectin and vascular cell adhesion molecule-1 on endothelial cells of hematopoietic tissues. Am J Pathol 148, 165-175.
Akashi, K., Traver, D., Miyamoto, T., and Weissman, I. L. (2000). A clonogenic common myeloid progenitor that gives rise to all myeloid lineages. Nature 404, 193. 10.1038/35004599.
Al-Amoodi, A. S., Li, Y., Al-Ghuneim, A., Allehaibi, H., Isaioglou, I., Esau, L. E., AbuSamra, D. B., and Merzaban, J. S. (2022). Refining the migration and engraftment of short-term and long-term HSCs by enhancing homing-specific adhesion mechanisms. Blood Adv 6, 4373-4391. 10.1182/bloodadvances.2022007465.
Benjamini, Y., Krieger, A. M., and Yekutieli, D. (2006). Adaptive linear step-up procedures that control the false discovery rate. Biometrika 93, 491-507. 10.1093/biomet/93.3.491.
Boggs, D. R. (1984). The total marrow mass of the mouse: A simplified method of measurement. American Journal of Hematology 16, 277-286. 10.1002/ajh.2830160309.
Buffone, A., Jr., Mondal, N., Gupta, R., McHugh, K. P., Lau, J. T., and Neelamegham, S. (2013). Silencing alphal,3-fucosyltransferases in human leukocytes reveals a role for FUT9 enzyme during E-selectin-mediated cell adhesion. The Journal of biological chemistry 288, 1620-1633. 10.1074/jbc.M112.400929.
Butcher, E. C. (1991). Leukocyte-endothelial cell recognition: three (or more) steps to specificity and diversity. Cell 67, 1033-1036. 10.1016/0092-8674(91)90279-8.
Copley, Michael R., Beer, Philip A., and Eaves, Connie J. (2012). Hematopoietic Stem Cell Heterogeneity Takes Center Stage. Cell Stem Cell 10, 690-697. https://doi.org/10.1016/j.stem.2012.05.006.
Dagia, N. M., Gadhoum, S. Z., Knoblauch, C. A., Spencer, J. A., Zamiri, P., Lin, C. P., and Sackstein, R. (2006). G-CSF induces E-selectin ligand expression on human myeloid cells. Nature medicine 12, 1185-1190. 10.1038/nm1470.
Dimitroff, C. J., Lee, J. Y., Rafii, S., Fuhlbrigge, R. C., and Sackstein, R. (2001). CD44 is a major E-selectin ligand on human hematopoietic progenitor cells. J Cell Biol 153, 1277-1286.
Doulatov, S., Notta, F., Eppert, K., Nguyen, L. T., Ohashi, P. S., and Dick, J. E. (2010). Revised map of the human progenitor hierarchy shows the origin of macrophages and dendritic cells in early lymphoid development. Nature Immunology 11, 585. 10.1038/ni.1889
https://www.nature.com/articles/ni.1889 #supplementary-information.
Dykstra, B., Lee, J., Mortensen, L. J., Yu, H., Wu, Z. L., Lin, C. P., Rossi, D. J., and Sackstein, R. (2016). Glycoengineering of E-Selectin Ligands by Intracellular versus Extracellular Fucosylation Differentially Affects Osteotropism of Human Mesenchymal Stem Cells. STEM CELLS 34, 2501-2511. 10.1002/stem.2435.
Enver, T., Pera, M., Peterson, C., and Andrews, P. W. (2009). Stem cell states, fates, and the rules of attraction. Cell Stem Cell 4, 387-397. 10.1016/j.stem.2009.04.011.
Eppert, K., Takenaka, K., Lechman, E. R., Waldron, L., Nilsson, B., van Galen, P., Metzeler, K. H., Poeppl, A., Ling, V., Beyene, J., et al. (2011). Stem cell gene expression programs influence clinical outcome in human leukemia. Nature medicine 17, 1086. 10.1038/nm.2415
https://www.nature.com/articles/nm.2415 #supplementary-information.
Frenette, P. S., Subbarao, S., Mazo, I. B., von Andrian, U. H., and Wagner, D. D. (1998). Endothelial selectins and vascular cell adhesion molecule-1 promote hematopoietic progenitor homing to bone marrow. Proceedings of the National Academy of Sciences 95, 14423-14428. 10.1073/pnas.95.24.14423.
Gadhoum, S. Z., and Sackstein, R. (2008). CD15 expression in human myeloid cell differentiation is regulated by sialidase activity. Nat Chem Biol 4, 751-757. 10.1038/nchembio.116.
Georgantas, R. W., Tanadve, V., Malehom, M., Heimfeld, S., Chen, C., Carr, L., Martinez-Murillo, F., Riggins, G., Kowalski, J., and Civin, C. I. (2004). Microarray and Serial Analysis of Gene Expression Analyses Identify Known and Novel Transcripts Overexpressed in Hematopoietic Stem Cells. Cancer Research 64, 4434-4441. 10.1158/0008-5472.can-03-3247.
Gooi, H. C., Thorpe, S. J., Hounsell, E. F., Rumpold, H., Kraft, D., Forster, O., and Feizi, T. (1983). Marker of peripheral blood granulocytes and monocytes of man recognized by two monoclonal antibodies VEP8 and VEP9 involves the trisaccharide 3-fucosyl-N-acetyllactosamine. Eur J Immunol 13, 306-312. 10.1002/eji.1830130407.
Griffin, M. E., and Hsieh-Wilson, L. C. (2016). Glycan Engineering for Cell and Developmental Biology. Cell Chem Biol 23, 108-121. 10.1016/j.chembiol.2015.12.007.
Handa, K., Stroud, M. R., and Hakomori, S.-i. (1997). Sialosyl-Fucosyl Poly-LacNAc without the Sialosyl-Lex Epitope as the Physiological Myeloid Cell Ligand in E-Selectin-Dependent Adhesion: Studies under Static and Dynamic Flow Conditions. Biochemistry 36, 12412-12420. 10.1021/bi971181t.
Hidalgo, A., and Frenette, P. S. (2005). Enforced fucosylation of neonatal CD34⁺ cells generates selectin ligands that enhance the initial interactions with microvessels but not homing to bone marrow. Blood 105, 567-575. 10.1182/blood-2004-03-1026.
Homeister, J. W., Thall, A. D., Petryniak, B., Malý, P., Rogers, C. E., Smith, P. L., Kelly, R. J., Gersten, K. M., Askari, S. W., Cheng, G., et al. (2001). The α(1,3)fucosyltransferases FucT-IV and FucT-VII Exert Collaborative Control over Selectin-Dependent Leukocyte Recruitment and Lymphocyte Homing. Immunity 15, 115-126. https://doi.org/10.1016/S1074-7613(01)00166-2.
Huang, D. W., Sherman, B. T., and Lempicki, R. A. (2008). Systematic and integrative analysis of large gene lists using DAVID bioinformatics resources. Nature Protocols 4, 44. 10.1038/nprot.2008.211
https://www.nature.com/articles/nprot.2008.211 #supplementary-information.
Huang, D. W., Sherman, B. T., and Lempicki, R. A. (2009). Bioinformatics enrichment tools: paths toward the comprehensive functional analysis of large gene lists. Nucleic acids research 37, 1-13. 10.1093/nar/gkn923.
Katayama, Y., Hidalgo, A., Furie, B. C., Vestweber, D., Furie, B., and Frenette, P. S. (2003). PSGL-1 participates in E-selectin-mediated progenitor homing to bone marrow: evidence for cooperation between E-selectin ligands and α4 integrin. Blood 102, 2060-2067. 10.1182/blood-2003-04-1212.
Kim, D., Langmead, B., and Salzberg, S. L. (2015). HISAT: a fast spliced aligner with low memory requirements. Nature Methods 12, 357. 10.1038/nmeth.3317
https://www.nature.com/articles/nmeth.3317 #supplementary-information.
Laird, D. J., von Andrian, U. H., and Wagers, A. J. (2008). Stem cell trafficking in tissue development, growth, and disease. Cell 132, 612-630. 10.1016/j.cell.2008.01.041.
Lanctot, P. M., Gage, F. H., and Varki, A. P. (2007). The glycans of stem cells. Current opinion in chemical biology 11, 373-380. 10.1016/j.cbpa.2007.05.032.
Lapidot, T., Dar, A., and Kollet, O. (2005). How do stem cells find their way home? Blood 106, 1901-1910. 10.1182/blood-2005-04-1417.
Love, M. I., Huber, W., and Anders, S. (2014). Moderated estimation of fold change and dispersion for RNA-seq data with DESeq2. Genome Biology 15, 550. 10.1186/s13059-014-0550-8.
Majeti, R., Park, C. Y., and Weissman, I. L. (2007). Identification of a Hierarchy of Multipotent Hematopoietic Progenitors in Human Cord Blood. Cell Stem Cell 1, 635-645. https://doi.org/10.1016/j.stem.2007.10.001.
Manz, M. G., Miyamoto, T., Akashi, K., and Weissman, I. L. (2002). Prospective isolation of human clonogenic common myeloid progenitors. Proceedings of the National Academy of Sciences 99, 11872-11877. 10.1073/pnas.172384399.
Mayani, H., Dragowska, W., and Lansdorp, P. (1993). Characterization of functionally distinct subpopulations of CD34+ cord blood cells in serum-free long-term cultures supplemented with hematopoietic cytokines. Blood 82, 2664-2672.
Mazo, I. B., Gutierrez-Ramos, J.-C., Frenette, P. S., Hynes, R. O., Wagner, D. D., and von Andrian, U. H. (1998). Hematopoietic Progenitor Cell Rolling in Bone Marrow Microvessels: Parallel Contributions by Endothelial Selectins and Vascular Cell Adhesion Molecule 1. The Journal of Experimental Medicine 188, 465-474. 10.1084/jem.188.3.465.
Merzaban, J. S., Burdick, M. M., Gadhoum, S. Z., Dagia, N. M., Chu, J. T., Fuhlbrigge, R. C., and Sackstein, R. (2011). Analysis of glycoprotein E-selectin ligands on human and mouse marrow cells enriched for hematopoietic stem/progenitor cells. Blood 118, 1774-1783. 10.1182/blood-2010-11-320705.
Miller, P. H., Knapp, D. J., and Eaves, C. J. (2013). Heterogeneity in hematopoietic stem cell populations: implications for transplantation. Current opinion in hematology 20, 257-264. 10.1097/MOH.0b013e328360aaf6.
Mondal, N., Buffone, A., Stolfa, G., Antonopoulos, A., Lau, J. T. Y., Haslam, S. M., Dell, A., and Neelamegham, S. (2015). ST3Gal-4 is the primary sialyltransferase regulating the synthesis of E-, P-, and L-selectin ligands on human myeloid leukocytes. Blood 125, 687-696. 10.1182/blood-2014-07-588590.
Mondal, N., Dykstra, B., Lee, J., Ashline, D. J., Reinhold, V. N., Rossi, D. J., and Sackstein, R. (2018). Distinct human α(1,3)-fucosyltransferases drive Lewis-X/sialyl Lewis-X assembly in human cells. Journal of Biological Chemistry 293, 7300-7314. 10.1074/jbc.RA117.000775.
Mondal, N., Stolfa, G., Antonopoulos, A., Zhu, Y., Wang, S.-S., Buffone, A., Atilla-Gokcumen, G. E., Haslam, S. M., Dell, A., and Neelamegham, S. (2016). Glycosphingolipids on Human Myeloid Cells Stabilize E-Selectin-Dependent Rolling in the Multistep Leukocyte Adhesion Cascade. Arteriosclerosis, Thrombosis, and Vascular Biology 36, 718-727. 10.1161/atvbaha.115.306748.
Muramatsu, T., and Muramatsu, H. (2004). Carbohydrate antigens expressed on stem cells and early embryonic cells. Glycoconjugate journal 21, 41-45. 10.1023/B:GLYC.0000043746.77504.28.
Nielsen, J. S., and McNagny, K. M. (2008). Novel functions of the CD34 family. Journal of Cell Science 121, 3683-3692. 10.1242/jcs.037507.
Nimrichter, L., Burdick, M. M., Aoki, K., Laroy, W., Fierro, M. A., Hudson, S. A., Von Seggern, C. E., Cotter, R. J., Bochner, B. S., Tiemeyer, M., et al. (2008). E-selectin receptors on human leukocytes. Blood 112, 3744-3752. 10.1182/blood-2008-04-149641.
Nonomura, C., Ando, H., Shinohara, H., Nojiri, H., Kikuchi, J., Nakamura, M., and Takaku, S. (2004). Not core 2 β1,6-N-acetylglucosaminyltransferase-2 or -3 but-1 regulates sialyl-Lewis x expression in human precursor B cells. Glycobiology 15, 271-280. 10.1093/glycob/cwi005.
Notta, F., Doulatov, S., Laurenti, E., Poeppl, A., Jurisica, I., and Dick, J. E. (2011). Isolation of Single Human Hematopoietic Stem Cells Capable of Long-Term Multilineage Engraftment. Science 333, 218-221. 10.1126/science.1201219.
Olweus, J., Lund-Johansen, F., and Terstappen, L. W. M. M. (1994). Expression of Cell Surface Markers during Differentiation of CD34+, CD38−/lo Fetal and Adult Bone Marrow Cells. ImmunoMethods 5, 179-188. https://doi.org/10.1006/immu.1994.1054.
Pachón-Peña, G., Donnelly, C., Ruiz-Cañada, C., Katz, A., Fernández-Veledo, S., Vendrell, J., and Sackstein, R. (2017). A Glycovariant of Human CD44 is Characteristically Expressed on Human Mesenchymal Stem Cells. Stem cells (Dayton, Ohio) 35, 1080-1092. 10.1002/stem.2549.
Peled, A., Grabovsky, V., Habler, L., Sandbank, J., Arenzana-Seisdedos, F., Petit, I., Ben-Hur, H., Lapidot, T., and Alon, R. (1999). The chemokine SDF-1 stimulates integrin-mediated arrest of CD34+ cells on vascular endothelium under shear flow. The Journal of Clinical Investigation 104, 1199-1211. 10.1172/JC17615.
Peled, A., Kollet, O., Ponomaryov, T., Petit, I., Franitza, S., Grabovsky, V., Slav, M. M., Nagler, A., Lider, O., Alon, R., et al. (2000). The chemokine SDF-1 activates the integrins LFA-1, VLA-4, and VLA-5 on immature human CD34+ cells: role in transendothelial/stromal migration and engraftment of NOD/SCID mice. Blood 95, 3289-3296.
Popat, U., Mehta, R. S., Rezvani, K., Fox, P., Kondo, K., Marin, D., McNiece, I., Oran, B., Hosing, C., Olson, A., et al. (2015). Enforced fucosylation of cord blood hematopoietic cells accelerates neutrophil and platelet engraftment after transplantation. Blood 125, 2885-2892. 10.1182/blood-2015-01-607366.
Prescher, J. A., and Bertozzi, C. R. (2006). Chemical technologies for probing glycans. Cell 126, 851-854. 10.1016/j.cell.2006.08.017.
Rix, B., Maduro, A. H., Bridge, K. S., and Grey, W. Markers for human hematopoietic stem cells: The disconnect between an identification marker and its function. Frontiers in Physiology (2022), doi: 10.3389/fphys.2022.1009160
Robinson, S. N., Simmons, P. J., Thomas, M. W., Brouard, N., Javni, J. A., Trilok, S., Shim, J.-S., Yang, H., Steiner, D., Decker, W. K., et al. (2012). Ex vivo fucosylation improves human cord blood engraftment in NOD-SCID IL-2Rγnull mice. Experimental Hematology 40, 445-456. https://doi.org/10.1016/j.exphem.2012.01.015.
Robinson, S. N., Thomas, M. W., Simmons, P. J., Lu, J., Yang, H., Parmar, S., Liu, X., Shah, N., Martin-Antonio, B., Bollard, C., et al. (2014). Fucosylation with fucosyltransferase VI or fucosyltransferase VII improves cord blood engraftment. Cytotherapy 16, 84-89. 10.1016/j.jcyt.2013.07.003.
Sackstein, R. (2004). The bone marrow is akin to skin: HCELL and the biology of hematopoietic stem cell homing. J Investig Dermatol 122, 1061-1069. 10.1111/j.0022-202X.2004.09301.x.
Sackstein, R. (2009). Glycosyltransferase-programmed stereosubstitution (GPS) to create HCELL: engineering a roadmap for cell migration. Immunological Reviews 230, 51-74. doi:10.1111/j.1600-065X.2009.00792.x.
Sackstein, R. (2016). Fulfilling Koch's postulates in glycoscience: HCELL, GPS and translational glycobiology. Glycobiology 26, 560-570. 10.1093/glycob/cww026.
Sackstein, R., Merzaban, J. S., Cain, D. W., Dagia, N. M., Spencer, J. A., Lin, C. P., and Wohlgemuth, R. (2008). Ex vivo glycan engineering of CD44 programs human multipotent mesenchymal stromal cell trafficking to bone. Nature medicine 14, 181-187. 10.1038/nm1703.
Sanada, C., Xavier-Ferrucio, J., Lu, Y.-C., Min, E., Zhang, P.-X., Zou, S., Kang, E., Zhang, M., Zerafati, G., Gallagher, P. G., and Krause, D. S. (2016). Adult human megakaryocyte-erythroid progenitors are in the CD34+CD38mid fraction. Blood 128, 923-933. 10.1182/blood-2016-01-693705.
Schopperle, W. M., and DeWolf, W. C. (2007). The TRA-1-60 and TRA-1-81 human pluripotent stem cell markers are expressed on podocalyxin in embryonal carcinoma. Stem cells (Dayton, Ohio) 25, 723-730. 10.1634/stemcells.2005-0597.
Schweitzer, K. M., Drager, A. M., van der Valk, P., Thijsen, S. F., Zevenbergen, A., Theijsmeijer, A. P., van der Schoot, C. E., and Langenhuijsen, M. M. (1996). Constitutive expression of E-selectin and vascular cell adhesion molecule-1 on endothelial cells of hematopoietic tissues. Am J Pathol 148, 165-175.
Sipkins, D. A., Wei, X., Wu, J. W., Runnels, J. M., Cote, D., Means, T. K., Luster, A. D., Scadden, D. T., and Lin, C. P. (2005). In vivo imaging of specialized bone marrow endothelial microdomains for tumour engraftment. Nature 435, 969-973.
Springer, T. A. (1994). Traffic signals for lymphocyte recirculation and leukocyte emigration: the multistep paradigm. Cell 76, 301-314. 10.1016/0092-8674(94)90337-9.
Stolfa, G., Mondal, N., Zhu, Y., Yu, X., Buffone, A., Jr., and Neelamegham, S. (2016). Using CRISPR-Cas9 to quantify the contributions of O-glycans, N-glycans and Glycosphingolipids to human leukocyte-endothelium adhesion. Scientific reports 6, 30392. 10.1038/srep30392.
Sutherland, H., Eaves, C., Eaves, A., Dragowska, W., and Lansdorp, P. (1989). Characterization and partial purification of human marrow cells capable of initiating long-term hematopoiesis in vitro. Blood 74, 1563-1570.
Tao, W., Wang, M., Voss, E. D., Cocklin, R. R., Smith, J. A., Cooper, S. H., and Broxmeyer, H. E. (2004). Comparative proteomic analysis of human CD34+ stem/progenitor cells and mature CD15+ myeloid cells. Stem cells (Dayton, Ohio) 22, 1003-1014. 10.1634/stemcells.22-6-1003.
Trumpp, A., and Haas, S. (2022). Cancer stem cells: The adventurous journey from hematopoietic to leukemic stem cells. Cell 185, 1266-1270. 10.1016/j.cell.2022.03.025.
Vandesompele, J., De Preter, K., Pattyn, F., Poppe, B., Van Roy, N., De Paepe, A., and Speleman, F. (2002). Accurate normalization of real-time quantitative R T-PCR data by geometric averaging of multiple internal control genes. Genome Biol 3, RESEARCH0034.
Weninger, W., Ulfman, L. H., Cheng, G., Souchkova, N., Quackenbush, E. J., Lowe, J. B., and von Andrian, U. H. (2000). Specialized contributions by alpha(1,3)-fucosyltransferase-IV and FucT-VII during leukocyte rolling in dermal microvessels. Immunity 12, 665-676.
Winkler, I. G., Barbier, V., Nowlan, B., Jacobsen, R. N., Forristal, C. E., Patton, J. T., Magnani, J. L., and Levesque, J.-P. (2012). Vascular niche E-selectin regulates hematopoietic stem cell dormancy, self renewal and chemoresistance. Nature medicine 18, 1651-1657. http://www.nature.com/nm/journal/v18/n11/abs/nm.2969.html #supplementary-information.
Wognum, B., Yuan, N., Lai, B., and Miller, C. L. (2013). Colony Forming Cell Assays for Human Hematopoietic Progenitor Cells. In Basic Cell Culture Protocols, C. D. Helgason, and C. L. Miller, eds. (Humana Press), pp. 267-283. 10.1007/978-1-62703-128-8_17.
Xia, L., McDaniel, J. M., Yago, T., Doeden, A., and McEver, R. P. (2004). Surface fucosylation of human cord blood cells augments binding to P-selectin and E-selectin and enhances engraftment in bone marrow. Blood 104, 3091-3096. 10.1182/blood-2004-02-0650.
Yang, W. H., Nussbaum, C., Grewal, P. K., Marth, J. D., and Sperandio, M. (2012). Coordinated roles of ST3Gal-VI and ST3Gal-IV sialyltransferases in the synthesis of selectin ligands. Blood 120, 1015-1026. 10.1182/blood-2012-04-424366.

1. A method for selecting one or more human hematopoietic stem/progenitor cells (HSPCs) from within a heterogenous population of lin⁻ HSPCs comprising:
contacting the heterogenous population of lin⁻ HSPCs with a binding molecule for sialylated Lewis X (sLeX);
measuring the amount of sLeX present on individual cells in the heterogenous population of lin⁻ HSPCs; and
selecting for one or more of sLeX^{high} cells based on the level of sLeX expression of the lin⁻ HSPCs, wherein the sLeX^{high} cells are the cells having the highest 15% sLeX expression level within the heterogenous population of sLeX+lin⁻ HSPCs.

2. The method of claim 1, further comprising the step of selecting for CD38− cells.

3. The method of claim 1, further comprising the step of selecting for CD34+ cells.

4. The method of claim 3, further comprising the step of selecting for CD38− cells.

5. The method of claim 1, further comprising the step of selecting for CD38+ cells.

6. The method of claim 5, further comprising the step of selecting for CD34+ cells.

7. The method of claim 1, wherein the heterogenous population of lin-HSPCs is from bone marrow, umbilical cord blood, adult (post-natal) blood, fetal blood, fetal liver, fetal spleen, embryonic yolk sac, embryonic ventral endothelium of dorsal aorta, adult (post-natal) liver, or adult (post-natal) spleen.

8. The method of claim 7, wherein the heterogenous population of lin-HSPCs are obtained by one or more steps of depleting differentiated HSPCs expressing lineage markers (i.e., depletion of lin⁺ nucleated cells)

9. The method of claim 2, wherein the selection for one or more of sLeX^{high} and CD38− cells comprises one or more steps of positive selection or negative selection.

10. The method of claim 1, wherein the selection of sLeX^{high} cells comprises one or more negative selection steps for depleting cells within the heterogenous population of sLeX+lin⁻ human HSPCs that express sLeX at density levels within the lower 85% of the range of sLeX expression within the heterogenous sLeX+lin⁻cell population.

11. The method of claim 1, wherein the selecting for one or more of sLeX^{high} cells comprises selecting for cells having the highest 10% of sLeX expression level within the heterogenous population of sLeX+lin⁻ cells.

12. The method of claim 1, wherein the selecting step comprises use of a molecule that binds the glycan determinant sLeX and the anti-determinant molecule contains a selection tag whereby cells bearing the anti-determinant molecule (e.g., anti-sLeX antibody, E-selectin-Ig chimera, etc.) can then be separated.

13. The method of claim 1, wherein the selecting step comprises use of molecule that binds the glycan determinant sLeX that carries a functional group to allow detection and separation of cells bearing the molecule-functional group, such as the use of magnetic bead-tagged anti-determinant molecules (e.g., magnetic bead-conjugated anti-sLeX antibody, magnetic bead-conjugated E-selectin-Ig chimera), biotin-tagged anti-determinant molecules (e.g., biotin-tagged anti-sLeX antibody, biotin-tagged E-selectin-Ig chimera), fluorescence-activated cell sorting (FACS) utilizing fluorochrome-tagged anti-determinant molecules (e.g., direct (one-step) using a fluorochrome-tagged anti-determinant molecules, or indirect (two-step) using a fluorochrome-tagged secondary reagent that recognizes the (primary) anti-determinant molecule (indirect (two-step) fluorochrome labelling of the cell)), chemically-modified anti-determinant molecules (e.g., anti-determinant molecule modified to contain a “clickable” chemical reagent such as an alkyne or azide modification), “panning” by affixing the anti-determinant molecules to a solid support matrix, passage of cells over affinity columns containing the anti-determinant molecules attached to beads or other matrices, or other techniques providing accurate cell separation.

14. A method for selecting one or more human hematopoietic stem/progenitor cells (HSPCs) from within a heterogenous population of lin⁻ HSPCs comprising:
contacting the heterogenous population of lin⁻ HSPCs with a binding molecule for sialylated Lewis X (sLeX);
measuring the amount of sLeX present on individual cells in the heterogenous population of lin⁻ HSPCs; and
selecting for one or more of sLeX^{low/-} cells based on the level of sLeX expression of the cells, wherein the sLeX^{low/-} subset comprises a fraction of 15% of the heterogenous population whose composition ranges from cells that lack sLeX expression to cells with the lowest level expression of sLeX (i.e., those cells that by FACS comprise the 15% fraction of the entire population that have the lowest fluorescence staining level for sLeX).

15. The method of claim 14, further comprising the step of selecting for CD38− cells.

16. The method of claim 14, further comprising the step of selecting for CD34+ cells.

17. The method of claim 16, further comprising the step of selecting for CD38− cells.

18. The method of claim 14, further comprising the step of selecting for CD38+ cells.

19. The method of claim 18, further comprising the step of selecting for CD34+ cells.

20. The method of claim 14, wherein the heterogenous population of lin-HSPCs is from bone marrow, umbilical cord blood, adult (post-natal) blood, fetal blood, fetal liver, fetal spleen, embryonic yolk sac, embryonic ventral endothelium of dorsal aorta, adult (post-natal) liver, or adult (post-natal) spleen.

21. The method of claim 17, wherein the selection for one or more of sLeX^{low/-}, CD34+, and CD38− cells comprises one or more steps of positive selection or negative selection to select for cells expressing the markers sLeX^{low/-}, CD34 and CD38.

22. The method of claim 14, wherein the heterogenous population of lin-HSPCs are obtained by one or more steps of depleting differentiated HSPCs expressing lineage markers (i.e., depletion of lin⁺ cells).

23. The method of claim 14, wherein the selection comprises one or more negative selection steps to enrich a population of sLeX^{low/-} cells by depletion of cells expressing sLeX at cell density levels >85% of the level within the heterogenous cell population.

24. The method of claim 14, wherein the sLeX^{low/-} cells comprises a fraction of 10% of the heterogenous population whose composition ranges from cells that lack sLeX expression to cells with the lowest level expression of sLeX.

25. The method of claim 14, wherein the selecting step comprises use of a molecule that binds the determinant (e.g., sLeX) and the anti-determinant molecule contains a selection tag whereby cells bearing the anti-determinant molecule (e.g., anti-sLeX antibody, E-selectin-Ig chimera, etc.) can then be separated.

26. The method of claim 14, wherein the selecting step comprises magnetic bead-tagged anti-determinant molecules (e.g., magnetic bead-conjugated anti-sLeX antibody, magnetic bead-conjugated E-selectin-Ig chimera), biotin-tagged anti-determinant molecules (e.g., biotin-tagged anti-sLeX antibody, biotin-tagged E-selectin-Ig chimera), fluorescence-activated cell sorting (FACS) utilizing fluorochrome-tagged anti-determinant molecules (e.g., direct (one-step) using a fluorochrome-tagged anti-determinant molecule, or indirect (two-step) using a fluorochrome-tagged secondary reagent that recognizes the (primary) anti-determinant molecules (indirect (two-step) fluorochrome labelling of the cell)), chemically-modified anti-determinant molecules (e.g., anti-determinant molecule modified to contain a “clickable” chemical reagent such as an alkyne or azide modification), “panning” by affixing the anti-determinant molecules to a solid support matrix, passage of cells over affinity columns containing the anti-determinant molecules attached to beads or other matrices, or other techniques providing accurate cell separation.

27.-45. (canceled)

46. A kit for enriching or isolating one or more human hematopoietic stem/progenitor cells (HSPCs) from within a heterogenous population of HSPCs comprising reagents for selecting one or more of the markers sLeX, CD38, and CD34, and instructions for use thereof.
